(19) United States
(12) Patent Application Publication
(10) Pub. No.: US 2023/0175069 A1
(43) Pub. Date: Jun. 8, 2023

(54) COMPOSITIONS AND METHODS FOR DETECTING GENE FUSIONS OF ESR1 AND CCDC170 FOR DETERMINING INCREASED RESISTANCE TO ENDOCRINE THERAPY AND FOR CANCER TREATMENT

(71) Applicant: UNIVERSITY OF PITTSBURGH-OF THE COMMONWEAL TH SYSTEM OF HIGHER EDUCATION, Pittsburgh, PA (US)

(72) Inventor: Xiaosong WANG, Sewickley, PA (US)

(21) Appl. No.: 17/922,027

(22) PCT Filed: Apr. 26, 2021
(86) PCT No.: PCT/US2021/029086

§ 371 Date: Oct. 28, 2022

Related U.S. Application Data
(60) Provisional application No. 63/017,312, filed on Apr. 29, 2020.

(51) Int. Cl. C12Q 1/6886; C12Q 1/6816; C07K 14/72; C07K 14/47; A61K 45/06

(52) U.S. Cl. CPC C12Q 1/6886; A61K 45/06; C07K 14/721; C07K 14/4738; C12Q 1/6816; C12Q 2600/106

(57) ABSTRACT

Disclosed herein are compositions and methods for detecting ESR1/CCDC170 gene fusions relating to cancer. Also disclosed herein are compositions and methods for diagnosing and treating cancers that include detecting an ESR1/CCDC170 gene fusion.

### CROSS-REFERENCE TO RELATED APPLICATIONS

This application claims the benefit of U.S. Provisional Application No. 63/017,312, filed Apr. 29, 2020, which is expressly incorporated herein by reference in its entirety.

### STATEMENT REGARDING FEDERALLY SPONSORED RESEARCH OR DEVELOPMENT

This invention was made with government support under grant numbers CA181368 and CA183976 awarded by the National Institutes of Health; and grant numbers W81XWH-12-1-0166, W81XWH-12-1-0167 and W81XWH-13-1-0431 awarded by United States Army Medical Research and Materiel Command. The government has certain rights in the invention.

### FIELD

The present disclosure relates to cancer treatment and diagnosis.

### BACKGROUND

Endocrine therapy is the most effective treatment for estrogen-receptor (ER) positive breast cancer. Agents targeting ER, including selective ER modulators (SERMs, such as Tamoxifen), selective ER down-regulators (SERDs, such as Fulvestrant) and aromatase inhibitors (AIs, such as letrozole) are the mainstay of treatment (Comprehensive molecular portraits of human breast tumours (2012); Giltnane JM (2017)). However, the efficacy of endocrine therapy is limited by intrinsic and acquired endocrine resistance (Musgrove EA (2009)). About quarter of patients with primary tumor and almost all patients with metastases will present with or eventually develop endocrine resistance (Ma CX (2015)). Tremendous effort has been made to investigate the mechanism of endocrine resistance, and emerging evidence now suggests that mutations or fusions of the ESR1 gene that encode ERα is one of the most important driving mechanisms (Ma CX (2015); Jeselsohn R (2015); Hartmaier RJ (2018)).

Recurrent gene fusions resulting from chromosome translocations are a critical class of genetic aberrations causing cancer (Mitelman F (2007)), which have fueled modern cancer therapeutics. Recently, several milestone studies have identified recurrent gene fusions in different types of solid tumors with tremendous clinical impact. This is exemplified by an EML4-ALK gene fusion identified in 3-5% of non-small-cell lung cancer patients that has led to a new precision medicine with stunning clinical responses (Koivunen JP (2018)). Most recently, The U.S. Food and Drug Administration granted accelerated approval to larotrectinib as the first pancancer drug for the treatment of solid tumors expressing NTRK gene fusions (Laetsch TW (2018)).

Previously a recurrent gene rearrangement was identified on chromosome 6 between ESR1 and the coiled-coil domain containing 170, CCDC170. The ESR1-CCDC170 fusion join the 5′ untranslated region of ESR1 to the coding region of CCDC170, generating N-terminally truncated CCDC170 proteins (ΔCCDC170) expressed under the ESR1 promoter. When introduced in breast cancer cells, ΔCCDC170 proteins led to increased cell invasiveness and tumorigenesis (Veeraraghavan J (2014)). To date, ESR1-CCDC170 remains the most frequent gene fusion (6%-8%) detected in the more-aggressive and endocrine-resistance form of breast cancer – luminal B breast cancer, and its recurrence has been subsequently supported by several other studies (Giltnane JM (2017); Hartmaier RJ (2018); Matissek KJ (2018); Fimereli D (2018); Lei JT (2018)). In addition, this fusion is also detected as a recurrent event in ovarian cancer, and its presence has been associated with exceptional short-term survival (Yang SYC (2018)). The association of ESR1- CCDC170 fusions with lack of response to neoadjuvant letrozole treatment (Giltnane JM (2017)).

While multiple mechanisms of resistance have been proposed such as loss of ER, dysregulation of ER co-regulators, and crosstalk with growth factor pathways (Britton DJ (2006), deGraffenried LA (2004), Drury SC (2011), Hutcheson IR (2003), Musgrove EA (2009), Osborne CK (2003), Osborne CK (2011), Parisot JP (1999), Zhou W (2014)), the genetic aberration causing endocrine resistance remains to be explored. What is needed are compositions and methods for determining the gene rearrangements associated with resistance to endocrine therapy in breast cancer patients. The compositions and methods disclosed herein address these and other needs.

### SUMMARY

It is shown herein that ESR1/CCDC170 fusions endow reduced endocrine sensitivity in luminal breast cancer cells in vitro and in vivo, via interactions with HER2, HER3, and SRC, and enhance activation of SRC and PI3K-AKT pathways. Accordingly, provide herein are new diagnostic and therapeutic strategies for breast tumors harboring ESR1/CCDC170 fusions, wherein, in some embodiments, the combination of inhibitors against these kinases and endocrine therapy are administered.

Provided herein are methods of diagnosing a subject with increased resistance to estrogen receptor antagonists (such as increased resistance to estrogen-receptor modulators, estrogen-receptor down-regulators, and/or aromatase inhibitors), comprising: obtaining a biological sample from the subject; and detecting an ESR1/CCDC170 (or ESR1-CCDC170) gene fusion in the sample, wherein the detection indicates the subject has increased resistance to estrogen receptor antagonists (such as increased resistance to estrogen-receptor modulators, estrogen-receptor down-regulators, and/or aromatase inhibitors) and the subject is diagnosed with increased resistance to estrogen receptor antagonists (such as increased resistance to estrogen-receptor modulators, estrogen-receptor down-regulators, and/or aromatase inhibitors). In some embodiments, the ESR1/CCDC170 gene fusion is selected from the group consisting of a E2-E2 fusion, a E2-E4 fusion, a E2-E5 fusion, a E2-E6 fusion, a E2-E7 fusion, a E2-E8 fusion, a E2-E10 fusion, and other fusion variations illustrated in FIG. 1C. In some aspects, the E2-E2 fusion comprises SEQ ID NO: 35, the E2-E4 fusion comprises SEQ ID NO: 36, the E2-E5 fusion comprises SEQ ID NO: 37, the E2-E6 fusion comprises SEQ ID NO: 38, the E2-E7 fusion comprises SEQ ID NO: 39, the E2-E8 fusion comprises SEQ ID NO: 40, and the E2-E10 fusion comprises SEQ ID NO: 41.

The method of detection can comprise contacting the biological sample with a reaction mixture comprising a probe specific for a fusion point in one of SEQ ID NO: 35, SEQ ID NO: 36, SEQ ID NO: 37, SEQ ID NO: 38, SEQ ID NO: 39, SEQ ID NO: 40 and SEQ ID NO: 41. The method of detection can alternatively or further comprise contacting the biological sample with a reaction mixture comprising two primers, wherein the first primer is complementary to a ESR1 polynucleotide sequence and the second primer is complementary to a CCDC170 polynucleotide sequence, wherein the ESR1/CCDC170 gene fusion is detectable by the presence of an amplicon generated by the first primer and the second primer. The method of detection can also comprise contacting the biological sample with a reaction mixture comprising two primers, wherein the first primer is complementary to a ESR1 polynucleotide sequence and the second primer is complementary to a CCDC 170 polynucleotide sequence, wherein hybridization of the two primers on a ESR1/CCDC170 gene fusion sequence provides a detectable signal, and the ESR1/CCDC170 gene fusion is detectable by the presence of the signal. In some embodiments, a first of the one or more primers is selected from the group consisting of SEQ ID NO: 42, and SEQ ID NO: 44, and a second of the one or more primers is selected from the group consisting of SEQ ID NO: 43 and SEQ ID NO: 45. In some embodiments, the primers are SEQ ID NO: 42 and SEQ ID NO: 43. In some embodiments, the primers are SEQ ID NO: 44 and SEQ ID NO: 45.

The methods described herein can be used to detect an ESR1/CCDC170 gene fusion in a subject that has a cancer, such as a breast cancer, including but not limited to a luminal B or metastatic breast cancer. The methods can further comprise administering to the subject a therapeutically effective amount of a HER inhibitor and/or a SRC inhibitor.

Also included herein are methods of treating a cancer in a subject comprising: detecting a ESR1/CCDC170 gene fusion in a sample obtained from the subject; and administering to the subject a therapeutically effective amount of a HER inhibitor and/or a SRC inhibitor and a therapeutically effective amount of an estrogen-receptor modulator, an estrogen-receptor down-regulator, and/or an aromatase inhibitor. The ESR1/CCDC170 gene fusion can be selected from the group consisting of a E2-E2 fusion, a E2-E4 fusion, a E2-E5 fusion, a E2-E6 fusion, a E2-E7 fusion, a E2-E8 fusion, a E2-E10 fusion, and other fusion variations illustrated in FIG. 1C. In some aspects, the E2-E2 fusion comprises SEQ ID NO: 35, the E2-E4 fusion comprises SEQ ID NO: 36, the E2-E5 fusion comprises SEQ ID NO: 37, the E2-E6 fusion comprises SEQ ID NO: 38, the E2-E7 fusion comprises SEQ ID NO: 39, the E2-E8 fusion comprises SEQ ID NO: 40, and the E2-E10 fusion comprises SEQ ID NO: 41.

Further included are methods for detecting an ESR1/CCDC170 gene fusion comprising: obtaining a biological sample from a subject; and detecting the fusion in the sample. In some embodiments, the detection can comprise contacting the biological sample with a reaction mixture comprising a probe specific for a fusion point sequence within one of SEQ ID NO: 35, SEQ ID NO: 36, SEQ ID NO: 37, SEQ ID NO: 38, SEQ ID NO: 39, SEQ ID NO: 40 and SEQ ID NO: 41. A detectable moiety can be covalently bonded to the probe, such as in a Nanostring assay. Kits comprising one or more probes are included, wherein each probe specifically hybridizes to a fusion point nucleotide sequence within a sequence selected from the group consisting of SEQ ID NO: 35, SEQ ID NO: 36, SEQ ID NO: 37, SEQ ID NO: 38, SEQ ID NO: 39, SEQ ID NO: 40 and SEQ ID NO: 41.

Further included are sequencing based methods such as transcriptome/genome sequencing methods or targeted sequencing for detecting an ESR1/CCDC170 gene fusion comprising: obtaining a biological sample from a subject; and detecting the fusion variants in the sample through transcriptome/genome sequencing methods or targeted sequencing and bioinformatics detection tools.

Further included are genomic DNA based detection methods including but not limit to genomic PCR, fluorescence in situ hybridization, or southern blots for detecting an ESR1/CCDC170 gene fusion comprising: obtaining a biological sample from a subject; and detecting the fusion variants in the sample through genomic PCR, fluorescence in situ hybridization, or southern blots.

Further included are protein-based methods known in the art, such as Mass spectrometry, immunohistochemistry, or western blot for detecting an ESR1-CCDC170 protein product comprising: obtaining a biological sample from a subject; and detecting the fusion variant proteins in the sample through Mass spectrometry, immunohistochemistry, or western blot.

### DESCRIPTION OF DRAWINGS

FIGS. 1(A-D). Schematics of ESR1-CCDC170 protein structure and the underlying genetic mechanism. (A) ESR1-CCDC170 fusion variants detected by RT-PCR in primary luminal breast cancer from Baylor College of Medicine (BCM) patient cohort (upper panel), and by RNAseq data from four metastatic breast cancer cohorts (lower panel). RNAseq data for metastatic breast cancer was analyzed using our fusion capture pipeline as we previously described (Veeraraghavan J (2014)). The positive cases are required to have at least three fusion reads with at least one junction read, or have at least two junction reads if the read lengths are more than 100 bp. The ER+ metastatic breast cancer cases that have metastatic samples are included in the RNAseq analysis. Of note, the fusion variants detected by RNAseq should be considered as potential variants as alternative splicing events can generate different ESR1-CCDC170 transcript variants from the same fusion variant junction as we previously described (Veeraraghavan J (2014)). (B) Schematic of the tandem duplication generating the ESR1-CCDC170 fusions (left) and the genomic break points identified in fusion positive tumors from BCM cohort. (C) Schematic of ESR1-CCDC170 fusion variants and the encoded proteins. CC, Coiled-coil; ABC: ATP-binding cassette, LCC, Low compositional complexity. The specific fusion variants are indicated on the left (i.e., E1/2-E9/10 indicates four fusion variants resulting from fusion of ESR1 exon 1 or 2 to CCDC170 exon 9 or 10). (D) Schematic showing the potential mechanism engaged by ESR1-CCDC170 to endow breast cancer cell survival and the druggable hypothesis.

FIGS. 2(A-C). ESR1-CCDC170 variants endow different degrees of reduced endocrine responses in vitro and in vivo. A) The responses of the T47D cells expressing a control vector, wtCCDC170, or ESR1-CCDC170 variants V2-5 to estrogen deprivation (ED) with or without 0.5 µM tamoxifen (Tam) as revealed by clonogenic assays. The relative cell growth was normalized to the vector control cells treated with vehicle (Means ± SD of triplicates). WT, wildtype CCDC170. P-values are based on student’s t-tests. B) Silencing of the E2-E10 fusion in HCC1428 cells reduced cell viability as shown by clonogenic assays. Cells were first treated with siRNAs for three days in biological triplicates, and then treated with 4-hydroxytamoxifen (Tam, 0.5 µM) or fulvestrant (Ful, 0.1 µM) together with the siRNAs simultaneously for 72 hours. The lower chart shows the relative intensity of triplicates (Means ± SD) normalized to the cells treated with vehicle and scramble siRNA control (siCtrl). For each of the measured protein, the relative density to GAPDH of vector (left box), E2-E7 (middle box), and E2-E10 (right box) is shown. The western blots and the representative images of clonogenic assays are shown on the top. **, P<0.01; Student’s t test. C) Kaplan-Meier curves for regression-free survival (time to tumor halving) of the engineered T47D xenograft tumors treated with tamoxifen. **P<0.01, ***P<0.001 (log-rank test).

FIG. 3. Verifying the ectopic expression of ESR1-CCDC170 fusion protein products and wtCCDC170 proteins in respective engineered T47D cells lines by Western Blots.

FIG. 4. ESR1-CCDC170 fusion variants endow tamoxifen resistance in vivo. The individual tumor growth curves of the T47D xenograft tumors expressing a vector, E2-E7 or E2-E10 ESR1-CCDC170 fusion variants with or without tamoxifen treatment (TAM). Athymic female mice prepped with estrogen (E2) pellets were injected with 1×10⁷ the engineered T47D cells, and randomized into with/without tamoxifen treatment groups when the tumor volume reached 150-200 mm³. Vector indicates a pLenti7.3 vector control.

FIGS. 5(A-C). Signaling alterations in the engineered T47D cells and HCC1428 cells following endocrine treatment in vitro and in vivo. A) Western blot analysis of the engineered T47D cells expressing ESR1-CCDC170 variants or wtCCDC170 (WT) cultured in normal medium (RPMI 1640 with phenol and 10% FBS), estrogen-deprived (ED) medium (phenol red-free RPMI 1640 with 5% CSS), or ED medium plus 4-OH tamoxifen (0.5 µM) for six days. B) Western blot analysis of protein extracts from the engineered T47D xenograft tumor tissues treated with tamoxifen. Densitometric results of Western blots are shown in the figure. *p<0.05,**p<0.01 (Student’s t-test). C) Western blot analysis of the HCC1428 cells treated with vehicle (veh), tamoxifen (Tam), or fulvestrant (Ful).

FIG. 6. Heat-map showing the top down regulated (p<0.1) signaling molecules in E2-E10-silenced HCC1428 cells revealed by RPPA data.

FIGS. 7(A-C). ESR1-CCDC170 forms homodimers, interacts with HER2/HER3/SRC complex, and confers sensitivity to HER2/SRC inhibitors in combination with tamoxifen. A) ESR1-CCDC170 interacts with HER2, HER3 and SRC as shown by immuno-precipitation assay of T47D cells expressing wtCCDC170 or E2-E10, performed with anti-HER2, HER3, or SRC antibody, and detected by immuno-blotting with the indicated antibodies. B) Subcellular localization of ESR1-CCDC170 and wtCCDC170 proteins in the engineered T47D cells. The nuclear and cytoplasmic proteins from the engineered T47D cells were first fractionated and followed by Western blot to detect the subcellular localization. C) BiFC assay to detect the dimerization of E2-E10 fusion protein. HT1080 cells were co-transfected to express indicated Yn-and Yc-tagged proteins. The histogram shows the percentage of YFP-positive cells with the reconstituted YFP signal detected by flow cytometry in the respective transfected HT1080 cells. Lower panel, Western blot using GFP antibodies that cross-identify the YFP portion of proteins verified that Yn- or Yc-tagged E2-E10 protein was expressed at comparable levels between different transfected HT1080 cells.

FIG. 8. Ectopically expressed V5-tagged ΔCCDC170 coprecipitates with HER2. Immunoprecipitation assay was performed with anti-V5 antibody in the T47D cells stably expressing V5-tagged E2-E7 or E2-E10, and the products were analyzed by immunoblotting with the indicated antibodies.

FIGS. 9(A-B). The therapeutic effect of lapatinib or dasatinib treatment in combination with tamoxifen in the engineered T47D cells. A) The responses of the T47D cells expressing a control vector, ESR1-CCDC170 variants, or wtCCDC170 to tamoxifen (0.5 µM), lapatinib (0.5 µM), or their combination as measured by clonogenic assays. The cells were cultured under estrogen-deprived condition in phenol red-free medium and treated with the indicated drugs for 15 days before fixation and staining. Upper panel, the representative images of clonogenic assays. Lower panel, the relative intensities normalized to the vector control cells treated with vehicle (Means ± SD of triplicates). B) Responses of the engineered T47D cells to 4-OH tamoxifen (0.5 µM), dasatinib (0.05 µM), or their combination as measured by clonogenic assay. *, P<0.05; ***P<0.001

FIGS. 10(A-B). SRC and HER2 inhibitors increased endocrine sensitivity of HCC1428 cells expressing endogenous ESR1-CCDC170 fusion. SRC and HER2 inhibitors increase endocrine sensitivity of HCC1428 cells expressing endogenous ESR1-CCDC170 fusion. A) Sensitivity of HCC1428 cells to tamoxifen in combination with selective targeted agents as measured by clonogenic assays. HCC1428 cells were treated with different dosages of 4-OH tamoxifen (µM), or 4-OH tamoxifen plus lapatinib (2 µM), dasatinib (0.05 µM), BEZ235 (8 nM), or AZD8931 (1 µM). Data were normalized against vehicle treatment alone (as 100%). B) Sensitivity of HCC1428 cells to fulvestrant in combination with selective targeted agents as measured by clonogenic assays. HCC1428 cells were treated with different dosages of fulvestrant (µM), or fulvestrant plus lapatinib (2 µM), dasatinib (0.05 µM), BEZ235 (8 nM), AZD8931 (1 µM), or lapatinib (2 µM) + dasatinib (0.05 µM). Data were normalized against vehicle treatment alone (as 100%). Tam, 4-OH tamoxifen; Ful, fulvestrant, Lapa, lapatinib; Dasa, dasatinib, BEZ, BEZ235, AZD, ZAD8931. ***P < 0.001 (two-way ANOVA).

FIGS. 11(A-B). The effects of HER2 or SRC silencing on endocrine sensitivity of HCC1428 cells expressing endogenous ESR1-CCDC170 fusion. A) The effect of HER2 silencing on the endocrine responsiveness of HCC1428 cells as shown by clonogenic assays. Western blots verifying the knockdown efficiency and the representative plate images are shown on the top. Intensities of colonies in each well were normalized to the control siRNA and vehicle treated group. For each of the treatment groups, the clonogenic viability of siCtrl (left bar), siHER2-1 (middle bar), and siHER2-2 (right bar) is shown. B) The effect of SRC silencing on the endocrine responsiveness of HCC1428 cells as shown by clonogenic assays. For each of the treatment groups, the clonogenic viability of siCtrl (left bar), siSRC-1 (middle bar), and siSRC-2 (right bar) is shown. Intensities of colonies in each well were normalized to the control siRNA and vehicle treated group. Tam, 4-OH tamoxifen (0.5 µM). Ful, Fulvestrant (0.1 µM). ***P < 0.001, *P < 0.05 (student’s t-test).

FIGS. 12(A-B). SRC and HER2 inhibitors increased endocrine sensitivity of ZR-75-1 cells expressing endogenous ESR1-CCDC170 fusion. A) Sensitivity of ZR-75-1 cells to tamoxifen in combination with HER2, SRC, or HER2+SRC inhibitors as measured by clonogenic assays. ZR-75-1 cells were treated with different dosages of 4-OH tamoxifen (µM), or 4-OH tamoxifen plus lapatinib (2 µM), dasatinib (0.05 µM), or lapatinib (2 µM) + dasatinib (0.05 µM). Data were normalized against vehicle treatment alone (as 100%). B) Sensitivity of ZR-75-1 cells to fulvestrant in combination with HER2, SRC, or HER2+SRC inhibitors as measured by clonogenic assays. ZR-75-1 cells were treated with different dosages of fulvestrant (µM), or fulvestrant plus lapatinib (2 µM), dasatinib (0.05 µM), or lapatinib (2 µM) + dasatinib (0.05 µM). Data were normalized against vehicle treatment alone (as 100%). Tam, 4-OH tamoxifen; Ful, fulvestrant, Lapa; lapatinib; Dasa, dasatinib.

FIG. 13. Western blots detecting HER2, HER3, and SRC protein expression in the cell line models used in the preliminary study. ***p<0.001 (two-way ANOVA).

FIG. 14. The tamoxifen-sensitizing effect of lapatinib in luminal breast cancer cell lines with or without ESR1-CCDC170 fusions. ESR1-CCDC170 positive breast cancer cell line HCC1428 and ZR-75-1, and fusion-negative cell line MDA-MB-415 and CAMA-1 were treated with 0.5 µM tamoxifen in combination with titrated dosages of Lapatinib (µM) for 10 to 13 days prior to fixation and staining. Left panel, the representative plate images of clonogenic assays. Right panel, the relative clonogenic viabilities normalized to tamoxifen monotreatment (Means ± SD. of triplicates).

### DETAILED DESCRIPTION

The findings disclosed herein include that ESR1-CCDC170 gene fusions result in increased resistance to estrogen receptor antagonists, and that patients harboring the ESR1-CCDC170 gene fusions are more effectively treated with HER2, HER3 and/or SRC inhibitors.

Accordingly, disclosed herein is a method for detecting ESR1/CCDC170 gene fusion. The fusion can be detected by contacting the sample with one or more primers specific for ESR1-CCDC170 fusion transcript or rearranged genomic DNA, performing an amplification reaction, and detecting an amplification product or amplicon. The fusion can also be detected by transcriptome or genome sequencing, or targeted sequencing, or Nanostring assay, or Fluorescence In Situ Hybridization, or Southern Blot. In some examples, the detection of the fusion indicates an increased resistance to tamoxifen in the subject. In some embodiments, the subject having a detected ESR1/CCDC170 gene fusion is administered with a therapeutically effective amount of a HER2, HER3 and/or SRC inhibitor. The subject can also be administered a therapeutically effective amount of tamoxifen or other estrogen receptor antagonist.

Terms used throughout this application are to be construed with ordinary and typical meaning to those of ordinary skill in the art. However, Applicants desire that the following terms be given the particular definition as provided below.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Amplifying,” “amplification,” and grammatical equivalents thereof refers to any method by which at least a part of a target nucleic acid sequence is reproduced in a template-dependent manner, including without limitation, a broad range of techniques for amplifying nucleic acid sequences, either linearly or exponentially. Exemplary means for performing an amplifying step include ligase chain reaction (LCR), ligase detection reaction (LDR), ligation followed by Qreplicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA)(TwistDx, Cambridg, UK), and self-sustained sequence replication (3SR), including multiplex versions or combinations thereof, for example but not limited to, OLA/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other places, Sambrook et al. Molecular Cloning, 3rd Edition; Ausbel et al.; PCR Primer: A Laboratory Manual, Diffenbach, Ed., Cold Spring Harbor Press (1995); The Electronic Protocol Book, Chang Bioscience (2002), Msuih et al., J. Clin. Micro. 34:501-07 (1996); The Nucleic Acid Protocols Handbook, R. Rapley, ed., Humana Press, Totowa, N.J. (2002).

“Administration” of “administering” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The term “biological sample” as used herein means a sample of biological tissue or fluid. Such samples include, but are not limited to, tissue isolated from animals. Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A “control” is an alternative subject or sample used in an experiment for comparison purposes. A control can be “positive” or “negative.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Accordingly, it should be understood that “encode” or “encoding”

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence (i.e., exon), non-coding sequence (e.g., intron), or control sequence, fragments thereof, and combinations thereof. The term “gene” or “gene sequence” includes, for example, control sequences upstream of the coding sequence (for example, the ribosome binding site).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” or “antagonist” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. Control samples (untreated with inhibitors) are assigned a relative activity value of 100%. Inhibition of a described target protein is achieved when the activity value relative to the control is about 80%, optionally 50% or 25, 10%, 5%, or 1% or less.

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides.

Unless otherwise specified, a “nucleotide sequence encoding an amino acid sequence” includes all nucleotide sequences that are degenerate versions of each other and that encode the same amino acid sequence. The nucleotide sequence that encodes a protein or an RNA may also include introns to the extent that the nucleotide sequence encoding the protein may in some version contain an intron(s).

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington’s Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “polynucleotide” refers to a single or double stranded polymer composed of nucleotide monomers. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The term “primer” or “amplification primer” refers to an oligonucleotide that is capable of acting as a point of initiation for the 5′ to 3′ synthesis of a primer extension product that is complementary to a nucleic acid strand. The primer extension product is synthesized in the presence of appropriate nucleotides and an agent for polymerization such as a DNA polymerase in an appropriate buffer and at a suitable temperature. The most widely used target amplification procedure is PCR, first described for the amplification of DNA by Muliis et al. in U.S. Pat. No. 4,683,195 and Mullis in U.S. Pat. No. 4,683,202 and is well known to those of ordinary skill in the art.

A “primer” or “primer sequence” hybridizes to a target nucleic acid sequence (for example, a DNA template to be amplified) to prime a nucleic acid synthesis reaction. The primer may be a DNA oligonucleotide, a RNA oligonucleotide, or a chimeric sequence. The primer may contain natural, synthetic, or modified nucleotides. Both the upper and lower limits of the length of the primer are empirically determined. The lower limit on primer length is the minimum length that is required to form a stable duplex upon hybridization with the target nucleic acid under nucleic acid amplification reaction conditions. Very short primers (usually less than 3-4 nucleotides long) do not form thermodynamically stable duplexes with target nucleic acids under such hybridization conditions. The upper limit is often determined by the possibility of having a duplex formation in a region other than the pre-determined nucleic acid sequence in the target nucleic acid. Generally, suitable primer lengths are in the range of about 10 to about 40 nucleotides long. In certain embodiments, for example, a primer can be 10-40, 15-30, or 10-20 nucleotides long. A primer is capable of acting as a point of initiation of synthesis on a polynucleotide sequence when placed under appropriate conditions. The primer will be completely or substantially complementary to a region of the target polynucleotide sequence to be copied. Therefore, under conditions conducive to hybridization, the primer will anneal to the complementary region of the target sequence. Upon addition of suitable reactants, including, but not limited to, a polymerase, nucleotide triphosphates, etc., the primer is extended by the polymerizing agent to form a copy of the target sequence. The primer may be single-stranded or alternatively may be partially double-stranded.

The term “primer pair” as used herein means a pair of oligonucleotide primers that are complementary to the sequences flanking a target sequence. The primer pair consists of a forward primer and a reverse primer. The forward primer has a nucleic acid sequence that is complementary to a sequence upstream, i.e., 5′ of the target sequence. The reverse primer has a nucleic acid sequence that is complementary to a sequence downstream, i.e., 3′ of the target sequence.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In some embodiments, administering an increased amount of an estrogen receptor antagonist is an administration that is increased by a percentage or fold listed above as compared to an amount previously administered to the subject or to an amount that is used in a general or study population under the same or similar circumstances.

The term “reduced”, “reduce”, “reduction”, “decrease”, or “decreased” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level.

“Reporter probe” refers to a molecule used in an amplification reaction, typically for quantitative or real-time PCR analysis, as well as end-point analysis. Such reporter probes can be used to monitor the amplification of the target nucleic acid sequence. In some embodiments, reporter probes present in an amplification reaction are suitable for monitoring the amount of amplicon(s) produced as a function of time. Such reporter probes include, but are not limited to, the 5′-exonuclease assay (e.g., U.S. Pat. No. 5,538,848) various stem-loop molecular beacons (see for example, U.S. Pat. Nos. 6,103,476 and 5,925,517), stemless or linear beacons (see, e.g., WO 99/21881), PNA MOLECULAR BEACONS (see, e.g., U.S. Pat. Nos. 6,355,421 and 6,593,091), linear PNA beacons, non-FRET probes (see, for example, U.S. Pat. No. 6,150,097), SUNRISE/AMPLIFLUOR probes (U.S. Pat. No. 6,548,250), stem-loop and duplex Scorpion probes (U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB ECLIPSE probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901. Reporter probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The terms “treat,” “treating,” “treatment,” and grammatical variations thereof as used herein, include partially or completely alleviating, mitigating or reducing the intensity of one or more attendant symptoms of a disorder or condition and/or alleviating or mitigating one or more causes of a disorder or condition. Treatments according to the invention may be applied pallatively or remedially.

Prophylactic administrations are given to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of a cancer.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition refers to an amount that is effective to achieve a desired therapeutic result. In some embodiments, a desired therapeutic result is a reduction of tumor size. In some embodiments, a desired therapeutic result is a reduction of cancer metastasis. In some embodiments, a desired therapeutic result is a reduction of a breast cancer, or a symptom of a breast cancer. In some embodiments, a desired therapeutic result is a reduction of a luminal B breast cancer, or a symptom thereof. In some embodiments, a desired therapeutic result is the prevention of cancer relapse. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as control of tumor growth. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

### Methods of Detecting, Diagnosing and Treating

Disclosed herein are methods of detecting an ESR1/CCDC170 gene fusion, said methods comprising obtaining a sample from a subject, and detecting whether the fusion is present in the sample. In some embodiments, an ESR1/CCDC170 gene fusion is detected in a sample derived from a subject having breast cancer and the detection indicates that the breast cancer has decreased sensitivity to an estrogen receptor antagonist (such as an estrogen-receptor modulator, an estrogen-receptor down-regulator, or an aromatase inhibitor). Accordingly, the present invention includes methods of diagnosing a breast cancer in a subject having decreased sensitivity to an estrogen receptor antagonist (such as an estrogen-receptor modulator, an estrogen-receptor down-regulator, or an aromatase inhibitor). In some examples, the estrogen-receptor modulator is tamoxifen, clomifene, raloxifene, or exemestane. In some examples, the estrogen-receptor down-modulator is fulvestrant. In some examples, the aromatase inhibitor is letrozole.

Also disclosed herein is a method of treating a breast cancer in a subject, said method comprising detecting an ESR1/CCDC170 gene fusion in a breast tissue sample obtained from the subject, and administering to the subject a therapeutically effective amount of a HER inhibitor and/or a SRC inhibitor. In some embodiments, an estrogen receptor antagonist is also administered to the subject, including administering an increased amount of an estrogen receptor antagonist.

As used herein, “gene fusion” refers to a chimeric genomic DNA resulting from the fusion of at least a portion of a first gene to a portion of a second gene. The point of transition between the sequence from the first gene in the fusion to the sequence from the second gene in the fusion is referred to as the “fusion point.” Transcription of the gene fusion results in a chimeric mRNA. Methods for detecting a gene fusion include detection of the chimeric genomic DNA, including by detection of the fusion point sequence within the chimeric genomic DNA, detection of the resultant chimeric mRNA, and detection of the resultant chimeric protein.

“ESR1” or “Estrogen Receptor 1” refers herein to a polypeptide that is involved in transducing signals following the activation of estrogen, and in humans, is encoded by the ESR1 gene. In some embodiments, the ESR1 polypeptide is that identified in one or more publicly available databases as follows: HGNC: 3467, Entrez Gene: 2099, Ensembl: ENSG00000091831, OMIM: 133430, UniProtKB: P03372. In some embodiments, the ESR1 polypeptide comprises the sequence of SEQ ID NO: 1, or a polypeptide sequence having at or greater than about 80%, about 85%, about 90%, about 95%, or about 98% homology with SEQ ID NO: 1, or a polypeptide comprising a portion of SEQ ID NO: 1. The ESR1 polypeptide of SEQ ID NO: 1 may represent an immature or pre-processed form of mature ESR1, and accordingly, included herein are mature or processed portions of the ESR1polypeptide in SEQ ID NO: 1.

The term “ESR1 polynucleotide” refers to a polynucleotide that encodes a ESR1 polypeptide, or any fragment thereof. In some embodiments, the ESR1 polynucleotide comprises an ESR1 exon 1 polynucleotide having a sequence of SEQ ID NO: 2, or a polynucleotide having at or greater than about 80%, about 85%, about 90%, about 95%, or about 98% homology with SEQ ID NO: 2, or a polynucleotide comprising a portion of SEQ ID NO: 2. In some embodiments, the ESR1 polynucleotide comprises a ESR1 exon 2 polynucleotide having a sequence of SEQ ID NO: 3, or a polynucleotide having at or greater than about 80%, about 85%, about 90%, about 95%, or about 98% homology with SEQ ID NO: 3, or a polynucleotide comprising a portion of SEQ ID NO: 3. In some embodiments, the ESR1 polynucleotide comprises a ESR1 exon 3 polynucleotide having a sequence of SEQ ID NO: 4, or a polynucleotide having at or greater than about 80%, about 85%, about 90%, about 95%, or about 98% homology with SEQ ID NO: 4, or a polynucleotide comprising a portion of SEQ ID NO: 4. In some embodiments, the ESR1 polynucleotide comprises a ESR1 exon 4 polynucleotide having a sequence of SEQ ID NO: 5, or a polynucleotide having at or greater than about 80%, about 85%, about 90%, about 95%, or about 98% homology with SEQ ID NO: 5, or a polynucleotide comprising a portion of SEQ ID NO: 5. In some embodiments, the ESR1 polynucleotide comprises a ESR1 exon 5 polynucleotide having a sequence of SEQ ID NO: 6, or a polynucleotide having at or greater than about 80%, about 85%, about 90%, about 95%, or about 98% homology with SEQ ID NO: 6, or a polynucleotide comprising a portion of SEQ ID NO: 6. In some embodiments, the ESR1 polynucleotide comprises a ESR1 exon 6 polynucleotide having a sequence of SEQ ID NO: 7, or a polynucleotide having at or greater than about 80%, about 85%, about 90%, about 95%, or about 98% homology with SEQ ID NO: 7, or a polynucleotide comprising a portion of SEQ ID NO: 7. In some embodiments, the ESR1 polynucleotide comprises a ESR1 exon 7 polynucleotide having a sequence of SEQ ID NO: 8, or a polynucleotide having at or greater than about 80%, about 85%, about 90%, about 95%, or about 98% homology with SEQ ID NO: 8, or a polynucleotide comprising a portion of SEQ ID NO: 8. In some embodiments, the ESR1 polynucleotide comprises a ESR1 exon 8 polynucleotide having a sequence of SEQ ID NO: 9, or a polynucleotide having at or greater than about 80%, about 85%, about 90%, about 95%, or about 98% homology with SEQ ID NO: 9, or a polynucleotide comprising a portion of SEQ ID NO: 9. In some embodiments, the ESR1 polynucleotide comprises a ESR1 exon 9 polynucleotide having a sequence of SEQ ID NO: 10, or a polynucleotide having at or greater than about 80%, about 85%, about 90%, about 95%, or about 98% homology with SEQ ID NO: 10, or a polynucleotide comprising a portion of SEQ ID NO: 10. In some embodiments, the ESR1 polynucleotide comprises a ESR1 exon 10 polynucleotide having a sequence of SEQ ID NO: 11, or a polynucleotide having at or greater than about 80%, about 85%, about 90%, about 95%, or about 98% homology with SEQ ID NO: 11, or a polynucleotide comprising a portion of SEQ ID NO: 11.

“CCDC170” or “Coiled-Coil Domain Containing 170” refers herein to a polypeptide that is encoded by the CCDC170 gene in human. In some embodiments, the CCDC170 polypeptide is that identified in one or more publicly available databases as follows: HGNC: 21177, Entrez Gene: 80129, Ensembl: ENSG00000120262, UniProtKB: Q8IYT3. In some embodiments, the CCDC170 polypeptide comprises the sequence of SEQ ID NO: 12 or a polypeptide sequence having at or greater than about 80%, about 85%, about 90%, about 95%, or about 98% homology with SEQ ID NO: 12, or a polypeptide comprising a portion of SEQ ID NO: 3. The CCDC170 polypeptide of SEQ ID NO: 12 may represent an immature or pre-processed form of mature CCDC170, and accordingly, included herein are mature or processed portions of the CCDC170 polypeptide in SEQ ID NO: 12.

The term “CCDC 170 polynucleotide” refers to a polynucleotide that encodes a CCDC170 polypeptide, or any fragment thereof. In some embodiments, the CCDC170 polynucleotide comprises an CCDC170 exon 1 polynucleotide having a sequence of SEQ ID NO: 13, or a polynucleotide having at or greater than about 80%, about 85%, about 90%, about 95%, or about 98% homology with SEQ ID NO: 13, or a polynucleotide comprising a portion of SEQ ID NO: 13. In some embodiments, the CCDC170 polynucleotide comprises a CCDC170 exon 2 polynucleotide having a sequence of SEQ ID NO: 14, or a polynucleotide having at or greater than about 80%, about 85%, about 90%, about 95%, or about 98% homology with SEQ ID NO: 14, or a polynucleotide comprising a portion of SEQ ID NO: 14. In some embodiments, the CCDC170 polynucleotide comprises a CCDC170 exon 3 polynucleotide having a sequence of SEQ ID NO: 15, or a polynucleotide having at or greater than about 80%, about 85%, about 90%, about 95%, or about 98% homology with SEQ ID NO: 15, or a polynucleotide comprising a portion of SEQ ID NO: 15. In some embodiments, the CCDC170 polynucleotide comprises a CCDC170 exon 4 polynucleotide having a sequence of SEQ ID NO: 16, or a polynucleotide having at or greater than about 80%, about 85%, about 90%, about 95%, or about 98% homology with SEQ ID NO: 16, or a polynucleotide comprising a portion of SEQ ID NO: 16. In some embodiments, the CCDC170 polynucleotide comprises a CCDC170 exon 5 polynucleotide having a sequence of SEQ ID NO: 17, or a polynucleotide having at or greater than about 80%, about 85%, about 90%, about 95%, or about 98% homology with SEQ ID NO: 17, or a polynucleotide comprising a portion of SEQ ID NO: 17. In some embodiments, the CCDC170 polynucleotide comprises a CCDC170 exon 6 polynucleotide having a sequence of SEQ ID NO: 18, or a polynucleotide having at or greater than about 80%, about 85%, about 90%, about 95%, or about 98% homology with SEQ ID NO: 18, or a polynucleotide comprising a portion of SEQ ID NO: 18. In some embodiments, the CCDC170 polynucleotide comprises a CCDC170 exon 7 polynucleotide having a sequence of SEQ ID NO: 19, or a polynucleotide having at or greater than about 80%, about 85%, about 90%, about 95%, or about 98% homology with SEQ ID NO: 19, or a polynucleotide comprising a portion of SEQ ID NO: 19. In some embodiments, the CCDC170 polynucleotide comprises a CCDC170 exon 8 polynucleotide having a sequence of SEQ ID NO: 20, or a polynucleotide having at or greater than about 80%, about 85%, about 90%, about 95%, or about 98% homology with SEQ ID NO: 20, or a polynucleotide comprising a portion of SEQ ID NO: 20. In some embodiments, the CCDC170 polynucleotide comprises a CCDC170 exon 9 polynucleotide having a sequence of SEQ ID NO: 21, or a polynucleotide having at or greater than about 80%, about 85%, about 90%, about 95%, or about 98% homology with SEQ ID NO: 21, or a polynucleotide comprising a portion of SEQ ID NO: 21. In some embodiments, the CCDC170 polynucleotide comprises a CCDC170 exon 10 polynucleotide having a sequence of SEQ ID NO: 22, or a polynucleotide having at or greater than about 80%, about 85%, about 90%, about 95%, or about 98% homology with SEQ ID NO: 22, or a polynucleotide comprising a portion of SEQ ID NO: 22. In some embodiments, the CCDC170 polynucleotide comprises a CCDC170 exon 11 polynucleotide having a sequence of SEQ ID NO: 23, or a polynucleotide having at or greater than about 80%, about 85%, about 90%, about 95%, or about 98% homology with SEQ ID NO: 23, or a polynucleotide comprising a portion of SEQ ID NO: 23.

It should be understood that the term “fusion” as used herein refers to a polynucleotide or polypeptide made by joining parts of two previously independent polynucleotides or polypeptides of ESR1 and CCDC170. In some embodiments, a fusion is formed by joining parts of two previously independent genes through translocation, interstitial deletion, or chromosomal inversion. Accordingly, “a fusion of a ESR1 polynucleotide sequence and a CCDC170 polynucleotide sequence” refers herein to a fusion of a ESR1 DNA sequence and a CCDC170 DNA sequence or a fusion mRNA transcribed from the fusion DNA. “ESR1/CCDC170 polynucleotide fusion” is used interchangeably herein with “fusion of a ESR1 polynucleotide sequence and a CCDC170 polynucleotide sequence.” “ESR1/CCDC170 fusion” refers to a “ESR1/CCDC170 polynucleotide fusion” and/or a “ESR1/CCDC170 polypeptide fusion.”

In some embodiments, the phrase “a fusion of a ESR1 polynucleotide sequence and a CCDC170 polynucleotide sequence” herein refers to a fusion of any ESR1 exon and any CCDC170 exon. In some embodiments, the fusion described herein is a fusion containing a fusion exon junction of any of the exons of ESR1 polynucleotide with any of the exons 2-11 (having at least a portion of exon 11) of a CCDC170 polynucleotide. In some embodiments, the fusion is: a fusion of exons 1-2 of a ESR polynucleotide with exons 2-11 of a CCDC170 polynucleotide (having at least a portion of exon 11) of a CCDC170 polynucleotide (referred to herein as an “E2-E2 fusion”); a fusion of exons 1-2 of a ESR1 polynucleotide with exons 4-11 (having at least a portion of exon 11) of a CCDC170 polynucleotide (referred to herein as an “E2-E6 fusion”); a fusion of exons 1-2 of a ESR1 polynucleotide with exons 5-11 (having at least a portion of exon 11) of a CCDC170 polynucleotide (referred to herein as an “E2-E6 fusion”); a fusion of exons 1-2 of a ESR1 polynucleotide with exons 6-11 (having at least a portion of exon 11) of a CCDC170 polynucleotide (referred to herein as an “E2-E6 fusion”); a fusion of exons 1-2 of a ESR1 polynucleotide with exons 8-11 (having at least a portion of exon 11) of a CCDC170 polynucleotide (referred to herein as an “E2-E8 fusion”); a fusion of exons 1-2 of a ESR1 polynucleotide with exons 10-11 (having at least a portion of exon 11) of a CCDC170 polynucleotide (referred to herein as an “E2-E10 fusion”).

The fusions described herein can be detected by contacting the sample with one or more primers specific for the fusion, performing an amplification reaction, and detecting an amplification product or amplicon. It should be understood and herein contemplated that the term “amplification reaction” of polynucleotide as used herein means the use of an amplification reaction (e.g., PCR) to increase the concentration of a particular nucleic acid sequence within a mixture of nucleic acid sequences. The term “PCR” as used herein refers to the polymerase chain reaction, a laboratory technique used to make multiple copies of a segment of a polynucleotide, as is well- known in the art. The term “PCR” includes all forms of PCR, such as real-time PCR, quantitative reverse transcription PCR (qRT-PCR), multiplex PCR, nested PCR, hot start PCR, or GC-Rich PCR. In some embodiments, the amplification reaction is real-time PCR. Exemplary procedures for real-time PCR can be found in “Quantitation of DNA/RNA Using Real-Time PCR Detection” published by Perkin Elmer Applied Biosystems (1999) and to PCR Protocols (Academic Press New York, 1989), incorporated by reference herein in their entireties. The amplification reaction can also be a loop-mediated isothermal amplification (LAMP), a reaction at a constant temperature using primers recognizing the distinct regions of target DNA for a highly specific amplification reaction. In some embodiments, the ESR1-CCDC170 polynucleotide fusion disclosed herein is detected by methods such as the Nanostring nCounter assay which directly measures target molecules without PCR amplification using ghost probes against one fusion partner gene, and reporter probes against the other fusion partner gene. In some embodiments, a fusion protein encoded by the fusion polynucleotide disclosed herein is detected by one or more protein detection assays including, for example, Western blotting, immunoblotting, ELISA, immunohistochemistry, or an electrophoresis method (e.g., SDS-PAGE).

The fusion can also be detected by any RNA or DNA, or protein-based methods known in the art, such as Nanostring assay or whole transcriptome, whole genome or targeted transcriptome or genome sequencing, or fluorescence in situ hybridization, or southern blot, or immunohistochemistry, or western blot.

In some embodiments, the one or more primers or Nanostring probes comprise a sequence selected from the group consisting of SEQ ID NO: 42, SEQ ID NO: 43, SEQ ID NO: 44, and SEQ ID NO: 45, or a polynucleotide sequence having at or greater than about 80%, about 85%, about 90%, about 95%, or about 98% homology with a sequence selected from the group consisting of SEQ ID NO: 42, SEQ ID NO: 43, SEQ ID NO: 44, and SEQ ID NO: 45, or a polynucleotide comprising a portion of a sequence selected from the group consisting of SEQ ID NO: 42, SEQ ID NO: 43, SEQ ID NO: 44, and SEQ ID NO: 45. In some embodiments, a first primer or Nanostring probe comprises a sequence selected from the group consisting of SEQ ID NO: 42 and SEQ ID NO:44, or a polynucleotide sequence having at or greater than about 80%, about 85%, about 90%, about 95%, about 98%, or about 99% homology with the sequence selected from SEQ ID NO: 42 and SEQ ID NO:44, or a polynucleotide comprising a portion of with the sequence selected from SEQ ID NO: 42 and SEQ ID NO:44, and second primer or Nanostring probe comprises a sequence selected from the group consisting of SEQ ID NO: 43 and SEQ ID NO:45, or a polynucleotide sequence having at or greater than about 80%, about 85%, about 90%, about 95%, about 98%, or about 99% homology with the sequence selected from SEQ ID NO: 43 and SEQ ID NO:45, or a polynucleotide comprising a portion of with the sequence selected from SEQ ID NO: 43 and SEQ ID NO:45.

As used herein, the term “detecting” refers to detection of a level of a fusion (e.g., the fusion of a ESR1 polynucleotide sequence and a CCDC170 polynucleotide) that is at least about 5% (e.g., at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1000%, at least about 2000%, at least about 3000%, or at least about 5000%) or at least about 5 times (e.g., at least about 6 times, at least about 7 times, at least about 8 times, at least about 9 times, at least about 10 times, at least about 20 times, at least about 30 times, at least about 40 times, at least about 50 times, or at least about 100 times) higher as compared to a sample from a subject in general or a study population (e.g., healthy control).

In certain embodiments, the primers are used in DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, regular PCR, real-time PCR, DNA sequencing, DNA extension, DNA polymerization, RNA transcription, and reverse transcription. Techniques and conditions that amplify the primer in a sequence specific manner are preferred. In certain embodiments, the primers are used for the DNA or RNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. In some embodiments, the primers are used for gene array analysis. Typically, the disclosed primers hybridize with a region of the disclosed nucleic acids (e.g., ESR1 or CCDC170) or they hybridize with the complement of the nucleic acids or complement of a region of the nucleic acids.

In some embodiments, subject has a cancer. The cancer can be any of breast cancer, prostate cancer, ovarian cancer, cervical cancer, skin cancer, pancreatic cancer, colorectal cancer, renal cancer, liver cancer, brain cancer, lymphoma, leukemia, and lung cancer. In certain aspects, the cancer is a breast cancer. In certain aspects, the cancer is a luminal A breast cancer. In certain aspects, the cancer is a luminal B breast cancer. It should be understood and herein contemplated that luminal A breast cancer refers to breast tumors that are estrogen receptor (ER) positive, progesterone receptor (PR) positive, and HER2 negative. Luminal B breast cancer refers to breast tumors that are estrogen receptor (ER) positive, progesterone receptor (PR) negative, and HER2 positive.

“ER” or “estrogen receptor” refers herein to receptors that are activated by estrogen. The two types of ERs include ERα and ERβ. ERα is encoded by the ESR1 gene, while ERβ is encoded by ESR2 gene. In some embodiments, the ESR2 polypeptide is that identified in one or more publicly available databases as follows: HGNC: 3468, Entrez Gene: 2100, Ensembl: ENSG00000140009, OMIM: 601663, UniProtKB: Q92731. In some embodiments, the ESR2 polypeptide comprises the sequence of SEQ ID NO: 24 or a polypeptide sequence having at or greater than about 80%, about 85%, about 90%, about 95%, or about 98% homology with SEQ ID NO: 24, or a polypeptide comprising a portion of SEQ ID NO: 24. The ESR2 polypeptide of SEQ ID NO: 24 may represent an immature or pre-processed form of mature ESR2, and accordingly, included herein are mature or processed portions of the ESR2 polypeptide in SEQ ID NO: 24.

“Progesterone receptor” or “PR” refers herein to a polypeptide is encoded by the PGR gene in human. In some embodiments, the PR polypeptide is that identified in one or more publicly available databases as follows: HGNC: 8910, Entrez Gene: 5241, Ensembl: ENSG00000082175, OMIM: 607311, UniProtKB: P06401. In some embodiments, the PR polypeptide comprises the sequence of SEQ ID NO: 25 or a polypeptide sequence having at or greater than about 80%, about 85%, about 90%, about 95%, or about 98% homology with SEQ ID NO: 25, or a polypeptide comprising a portion of SEQ ID NO: 25. The PR polypeptide of SEQ ID NO: 25 may represent an immature or pre-processed form of mature PR, and accordingly, included herein are mature or processed portions of the PR polypeptide in SEQ ID NO: 25.

The “sample” referred to herein is a fluid or tissue sample. In some embodiments, the sample is a breast tissue sample. In some embodiments, the breast tissue is cancerous. Included herein are methods that comprise detection of an increased amount of the ESR1/CCDC170 fusion in a breast tissue sample as compared to a control, wherein the control can be a normal breast tissue or any normal tissue other than testis tissue, and wherein the control can be obtained from the same subject or a different subject. In some embodiments, the control is a level or amount of the ESR1/CCDC170 fusion in a general or study population. In some embodiments, the cancerous breast tissue exhibits an increased amount of the fusion of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a control, or at least about a 2-fold, or at least about a 3-fold, or at least about a 4-fold, or at least about a 5-fold, or at least about a 10-fold, at least about a 20-fold, at least about a 50-fold, at least about a 100-fold, at least about a 500-fold, or at least about a 1000-fold as compared to a control.

It should be understood and herein contemplated that detection of the ESR1/CCDC170 fusion or an increase in the amount of the ESR1/CCDC170 fusion as compared to a control indicates a decreased sensitivity of the tissue sample, cancer cell or tumor to an estrogen receptor antagonist (such as an estrogen-receptor modulator, an estrogen-receptor down-regulator, or an aromatase inhibitor).

It should be understood that the term “estrogen-receptor modulator” herein refers to a class of drugs that bind with estrogen receptors. In some examples, the estrogen-receptor modulator acts as an agonist to enhance estrogen activity. In some examples, the estrogen-receptor modulator acts as an antagonist to inhibit estrogen activity. In some examples, the estrogen-receptor modulator is selected from tamoxifen, clomifene, raloxifene, and exemestane. In some embodiments, the estrogen-receptor modulator is tamoxifen.

As used herein, “tamoxifen” refers to a composition having the below chemical structure.

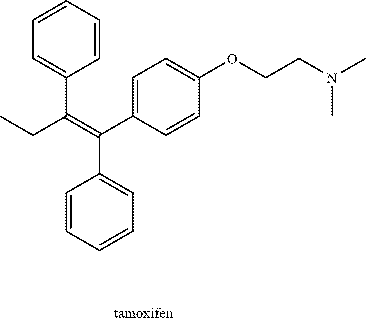

As used herein, “clomifene” refers to a composition having the below chemical structure.

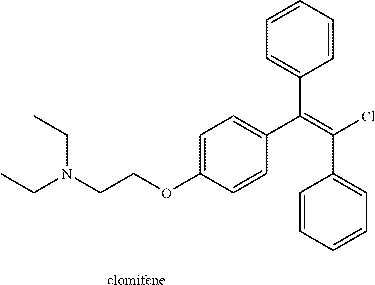

As used herein, “raloxifene” refers to a composition having the below chemical structure.

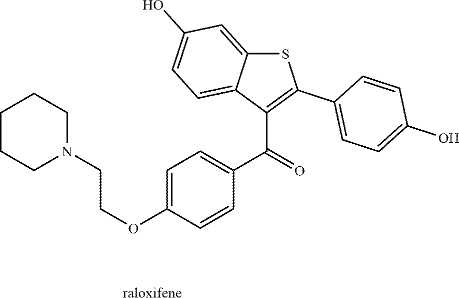

As used herein, “exemestane” refers to a composition having the below chemical structure.

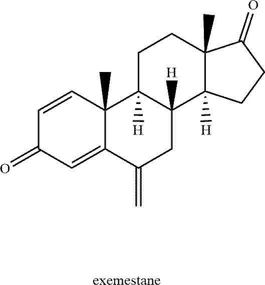

The term “estrogen-receptor down-modulator” or “estrogen-receptor down-regulator” herein refers to a class of drugs that bind to estrogen receptors and cause the receptor to be degraded. In some examples, the estrogen-receptor down-modulator used herein is fulvestrant.

As used herein, “fulvestrant” refers to a composition having the below chemical structure.

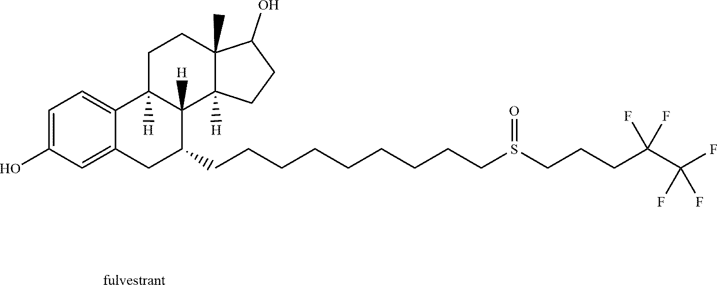

Aromatase inhibitors work by blocking the enzyme aromatase, which turns the hormone androgen into estrogen in the body. In some examples, the aromatase inhibitor is letrozole. As used herein, “letrozole” refers to a composition having the below chemical structure.

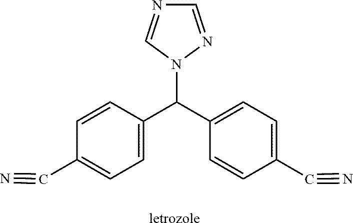

The ESR1/CCDC170 gene fusion can be detected using any method described herein. In some embodiments, the decreased sensitivity of a cancer cell or tumor refers to a more significant increase in tumor growth, a larger increase in tumor volume or size, a slower clearance of tumor, a decrease in cancer cell death, an increase in cell migration, metastasis, and/or proliferation as compared to a control cancer cell or tumor, wherein the control tumor or cancer cell does not have the ESR1/CCDC170 fusion disclosed herein. In some embodiments, the tumor or cancer cell comprising the ESR1/CCDC170 fusion exhibits a decreased sensitivity to an estrogen receptor antagonist (such as an estrogen-receptor modulator, an estrogen-receptor down-regulator, or an aromatase inhibitor) of at least about at least about 10%, at least about 20%, or at least about 30%, or at least about 40%, or at least about 50%, or at least about 60%, or at least about 70%, or at least about 80%, or at least about 90% or at least about 100%, or a decreased sensitivity to an estrogen receptor antagonist (such as an estrogen-receptor modulator, an estrogen-receptor down-regulator, or an aromatase inhibitor) of at least about a 2-fold, or at least about a 3-fold, or at least about a 4-fold, or at least about a 5-fold, or at least about a 10-fold, at least about a 20-fold, at least about a 50-fold, at least about a 100-fold, or at least about a 500-fold as compared to a control.

In some embodiments, detection of the ESR1/CCDC170 fusion or an increase in the amount of the ESR1/CCDC170 fusion as compared to a control indicates a decreased sensitivity of the tissue sample, cancer cell or tumor to an estrogen receptor antagonist.

Since detection of a ESR1/CCDC170 fusion indicates an increased resistance to an estrogen receptor antagonist (such as an estrogen-receptor modulator, an estrogen-receptor down-regulator, or an aromatase inhibitor), or a decrease in the effectiveness of an estrogen receptor antagonist (such as an estrogen-receptor modulator, an estrogen-receptor down-regulator, or an aromatase inhibitor) in the subject, certain embodiments further include treating the subject with a therapeutically effective amount of a HER inhibitor (e.g., a HER2 inhibitor) and/or a SRC inhibitor. The administration of the HER inhibitor and/or the SRC inhibitor can be in addition to administration of an estrogen receptor antagonist or an increased amount of an estrogen receptor antagonist.

“HER” refers to any of ErbB1/EGFR/HER1, ErbB2/HER2, ErbB3/HER3, and ErbB4/HER4, which are members of the ErbB receptor tyrosine kinase family. As used herein, “HER inhibitor” refers to a composition that inhibits the tyrosine kinase activity and/or intracellular signaling of a HER molecule. In some embodiments, the HER molecule is a HER2, a HER3, or a HER2/HER3 heterodimer. “HER2” “Erb-B2 Receptor Tyrosine Kinase 2” refers herein to a polypeptide encoded by the ERBB2 gene in humans. In some embodiments, the HER2 polypeptide is that identified in one or more publicly available databases as follows: HGNC: 3430, Entrez Gene: 2064, Ensembl: ENSG00000141736, OMIM: 164870, UniProtKB: P04626. In some embodiments, the HER2 polypeptide comprises the sequence of SEQ ID NO: 26 or a polypeptide sequence having at or greater than about 80%, about 85%, about 90%, about 95%, or about 98% homology with SEQ ID NO: 26, or a polypeptide comprising a portion of SEQ ID NO: 26. The HER2 polypeptide of SEQ ID NO: 26 may represent an immature or pre-processed form of mature HER2, and accordingly, included herein are mature or processed portions of the HER2 polypeptide in SEQ ID NO: 26. “HER3” or “Erb-B2 Receptor Tyrosine Kinase 3” refers herein to a polypeptide encoded by the ERBB3 gene in humans. In some embodiments, the HER2 polypeptide is that identified in one or more publicly available databases as follows: HGNC: 3431, Entrez Gene: 2065, Ensembl: ENSG00000065361, OMIM: 190151, UniProtKB: P21860. In some embodiments, the HER3 polypeptide comprises the sequence of SEQ ID NO: 27 or a polypeptide sequence having at or greater than about 80%, about 85%, about 90%, about 95%, or about 98% homology with SEQ ID NO: 27, or a polypeptide comprising a portion of SEQ ID NO: 27. The HER3 polypeptide of SEQ ID NO: 27 may represent an immature or pre-processed form of mature HER3, and accordingly, included herein are mature or processed portions of the HER3 polypeptide in SEQ ID NO: 27.

In some embodiments, the HER2 inhibitor is lapatinib. The term “lapatinib” refers to a composition having the below chemical structure.

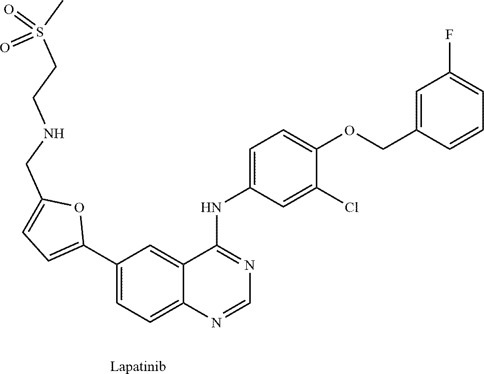

In some embodiments, the HER2 inhibitor is neratinib. The term “neratinib” refers to a composition having the below chemical structure.

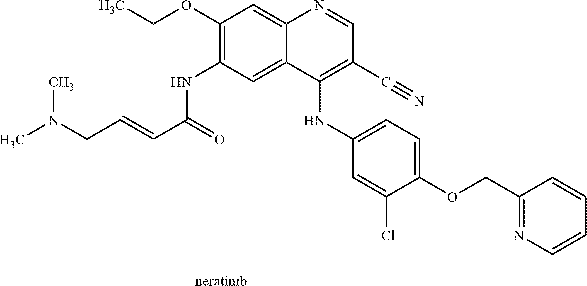

In some embodiments, the HER2 inhibitor is trasuzumab. In some embodiments, the HER2 inhibitor is persuzumab. In some emobidments, the HER2 inhibitor is selected from the group consisting of lapatinib, neratinib, trasuzumab and persuzumab.

In some embodiments, the HER3 inhibitor is Patritumab, Seribantumab, Elgemtumab, KTN3379, AV-203, or GSK2849330.

“SRC” of “SRC Proto-Oncogene, Non-Receptor Tyrosine Kinase” refers herein to a polypeptide that is encoded by the SRC gene in humans. In some embodiments, the SRC polypeptide is that identified in one or more publicly available databases as follows: HGNC: 11283, Entrez Gene: 6714, Ensembl: ENSG00000197122, OMIM: 190090, UniProtKB: P12931. In some embodiments, the SRC polypeptide comprises the sequence of SEQ ID NO: 28 or a polypeptide sequence having at or greater than about 80%, about 85%, about 90%, about 95%, or about 98% homology with SEQ ID NO: 28, or a polypeptide comprising a portion of SEQ ID NO: 28. The SRC polypeptide of SEQ ID NO: 28 may represent an immature or pre-processed form of mature SRC, and accordingly, included herein are mature or processed portions of the SRC polypeptide in SEQ ID NO: 28. As used herein, “SRC inhibitor” refers to a composition that inhibits the tyrosine kinase activity and/or intracellular signaling of a SRC molecule.

In some embodiments, the SRC inhibitor is dasatinib. The term “dasatinib” refers to a composition having the below chemical structure.

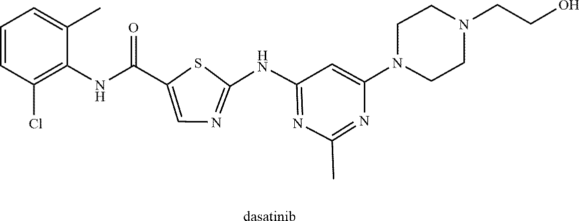

In other embodiments, the SRC inhibitor is bosutinib, ponatinib, vandetanib, or saracatninib.

As the timing of a cancer can often not be predicted, it should be understood the disclosed methods of treating, preventing, reducing, and/or inhibiting the disease or disorder described herein can be used prior to or following the onset of the disease or disorder, to treat, prevent, inhibit, and/or reduce the disease or disorder or symptoms thereof. In one aspect, the disclosed methods can be employed 30, 29, 28, 27, 26, 25, 24, 23, 22, 21, 20, 19, 18, 17, 16, 15, 14, 13, 12, 11, 10, 9, 8, 7, 6, 5, 4, 3, 2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to onset of the disease or disorder; or 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months, 2, 3, 4, 5, 6, 7, 8, 9, 10, 15, 20, 25, 30, 35, 40, 45, 50, 55, 60 or more years after onset of the disease or disorder.

Dosing frequency for the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 min, once every 30 min, once every 20 min, or once every 10 min. Administration can also be continuous and adjusted to maintaining a level of the compound within any desired and specified range.

Administration of any combination of a HER inhibitor, a SRC inhibitor and an estrogen receptor antagonist can occur simultaneously, or non-simultaneously in an order. In some embodiments, a HER inhibitor and/or a SRC inhibitor are administered simultaneously with an estrogen receptor antagonist. In other embodiments, a HER inhibitor and/or a SRC inhibitor are administered before an estrogen receptor antagonist. In other embodiments, a HER inhibitor and/or a SRC inhibitor are administered after an estrogen receptor antagonist.

### Kits

Included herein are kits comprising a probe or a set of probes, for example, a detectable probe or a set of amplification primers that specifically recognize a nucleic acid comprising a fusion point or break point. The kit can further include, in the same vessel, or in a separate vessel, a component from an amplification reaction mixture, such as a polymerase, typically not from human origin, dNTPs, and/or UDG. In some embodiments, the amplification primers are selected from the group consisting of SEQ ID NO: 42, SEQ ID NO: 43, SEQ ID NO: 44, and SEQ ID NO: 45. In some embodiments, the detectable probe is selected from polynucleotide sequence that specifically hybridizes to a fusion point nucleotide sequence within SEQ ID NO: 35, SEQ ID NO: 36, SEQ ID NO: 37, SEQ ID NO: 38, SEQ ID NO:39, SEQ ID NO:40 or SEQ ID NO:41. In some embodiments, the kit comprises a detectable moiety that is covalently bonded to the probe. Furthermore, the kit can include a control nucleic acid. For example, the control nucleic acid can include a sequence that includes a fusion point sequence within a sequence selected from the group of SEQ ID NO: 35, SEQ ID NO: 36, SEQ ID NO: 37, SEQ ID NO: 38, SEQ ID NO:39, SEQ ID NO:40 and SEQ ID NO:41.

All patents, patent applications, and publications referenced herein are incorporated by reference in their entirety for all purposes.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Compositions and Methods for Detecting Gene Fusions of ESR1 and CCDC170 For Determining Increased Resistance to Endocrine Therapy and Increased Sensitivity to HER2/HER3/SRC Inhibition

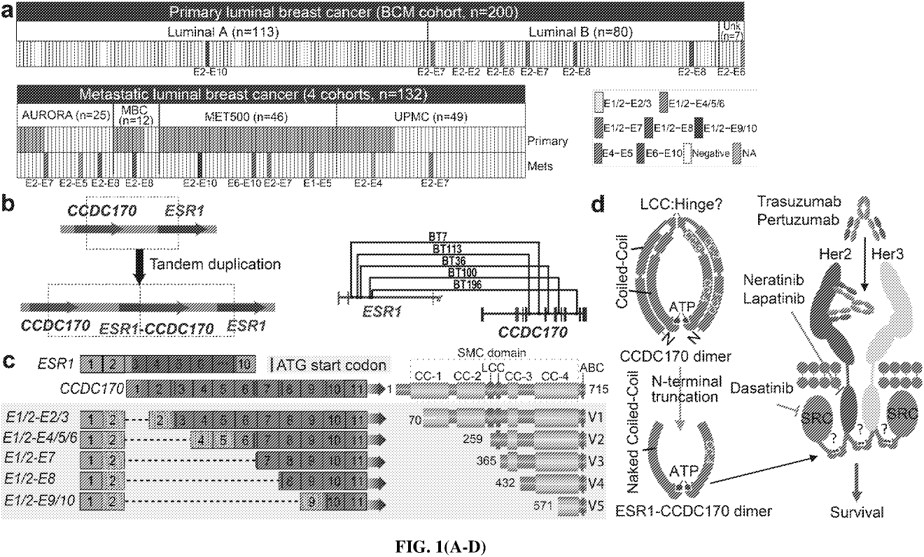

Emerging evidence shows that ESR1 mutations or fusions constitute the most common genetic mechanisms for endocrine resistance, accounting for approximately 20-50% of advanced breast cancers (Ma CX (2015), Jeselsohn R (2015), Hartmaier RJ (2018), Lei JT (2019), Giltnane JM (2017), Robinson DR (2013)). A previous study discovered the first and most prevalent form of ESR1 fusions generated by localized rearrangements between ESR1 and its immediate centromeric neighbor gene coiled-coil domain containing 170 (CCDC170), a majority of which are tandem duplications (FIGS. 1A-1B) (Veeraraghavan J, 2014). ESR1-CCDC170 fusions are preferentially detected in approximately8% of ER-positive HER2-negative luminal B breast cancers (FIG. 1A). Luminal B is a more aggressive luminal breast cancer form characterized by early relapse following endocrine therapy and high risk of metastatic dissemination, accounting for about one third of ER positive breast cancers (Yersal O, 2014). Distinct from other ESR1 fusions that preserve the exon 1-6 of ESR1 containing the ER transcriptional activation domain (Lei JT), most ESR1-CCDC170 fusions join the 5′ untranslated region (UTR) of ESR1 to the coding region of CCDC170. While a wide-variety of fusion variants have been observed, these fusions encode only five distinct forms of amino(N)-terminally truncated CCDC170 proteins (ΔCCDC170) due to the five silent ATG start codons present within the CCDC170 open-reading frame (ORF) (FIG. 1C). The expression of the fusion is driven by the ESR1 promoter. CCDC170 belongs to a structural maintenance of chromosome (SMC) protein family that maintains chromosome conformation through SMC-domain dependent looping and microtubule stabilization during interphase and mitosis (Schalbetter SA (2017)), Laflamme G (2014)). Genetic variants of CCDC170 have been reported to be strong breast cancer risk factors (Dunning AM (2016), Hong Y (2014)). ESR1-CCDC170 fusions lead to different degrees of deletion of the SMC domain, but retain a putative high-affinity ATP-binding pocket at C-terminus.

The previous data show that, ESR1-CCDC170 fusions endow ligand-independent growth factor signaling, leading to increased cell motility, invasion, anchorage-independent growth, and reduced endocrine sensitivity in vitro, as well as enhanced tumor formation in vivo (Veeraraghavan J, 2014). To date, ESR1-CCDC170 remains the most frequent pathological gene fusion detected in luminal breast cancer, and its recurrence has been subsequently supported by several recent studies (Hartmaier RJ (2018), Giltnane JM (2017), Matissek KJ (2018), Fimereli D (2018), Lei JT (2018)). In addition to breast cancer, a recent publication reported ESR1-CCDC170 as a recurrent event in ovarian cancer associated with exceptional short-term survival (Yang SYC (2018)). Nonetheless, detailed functional evidence demonstrating the causal role of ESR1-CCDC170 in endocrine resistance especially in the in vivo and clinical context, and the precise mechanism for this fusion to endow ligand-independent growth factor signaling has been lacking. Here the data show that ESR1-CCDC170 fusions endow breast cancer cell survival and reduced endocrine sensitivity in vitro and in vivo via physically binding to the HER2/HER3/SRC complex and activate their downstream signaling. Furthermore, the RNAseq data from four metastatic breast cancer patient cohorts were analyzed including University of Michigan cohort (MET500) (Robinson DR, 2017), Count me in Metastatic Breast Cancer Project (MBC) (Nikhil Wagle CP, 2018), UPMC cohort, and the BCRF AURORA project (Zardavas D, 2014). These analyses detected ESR1-CCDC170 fusions in ~8% of metastatic luminal breast cancers (FIG. 1A). More important, these experimental data show that ESR1-CCDC170 appears to engender a special exploitable vulnerability in these deadly tumors that can be attacked by HER2/HER3/SRC inhibitors, which can have dramatic clinical implications.

An independent report in Science Translational Medicine assessed ESR1 fusions in surgical samples from stage I-III ER positive and HER2 negative breast cancer patients following neoadjuvant letrozole treatment (Giltnane JM, 2017). This study detected ESR1-CCDC170 in three out of 27 endocrine resistant or intermediate resistant tumors (11%), among which two tumors harbor E2-E6 variants, and one tumor harbors E2-E8 variant. In addition, they also detected ESR1-CCDC170 in one out of 29 endocrine sensitive tumors. This case however, express a E4-E5 variant encoding a c-terminally truncated ESR1 protein that disrupts the DNA binding domain, thus is likely nonfunctional. This shows that fusion variants V2 and V4 are associated with lack of response to neoadjuvant letrozole treatment, and that truncation of CCDC170 that removes CC1 and CC2 domains are sufficient to confer reduced endocrine sensitivity. Furthermore, another clinical study reported in 2019 SABCS meeting detected ESR1-CCDC170 in the primary tumors of HER2-negative luminal breast cancer patients that experienced metastatic relapse. ESR1-CCDC170 fusions are detected in 29 out of 307 patients (9.4%) which significantly correlate with worse disease-free survival following initial surgery (AM Sieuwerts SV, 2019). The most relevant fusion variants involve ESR1 E2 and CCDC170 E4-10 (V2-5).

Recurrent gene fusions have laid the foundation for modern cancer therapeutics (Mitelman F, 2007). In addition to leukemia, several milestone studies have identified recurrent fusions in multiple solid tumors that led to therapeutic breakthroughs. The most prominent examples are EML4-ALK and ROS1 fusions in 3%-5% and 1%-2% of non-small cell lung cancer, respectively, FGFR-TACC fusion in approximately3% of glioblastomas, and NTRK fusions detected in approximately 0.3% of tumors, all of which are matched with stunningly effective targeted therapies (Koivunen JP (2008), Singh D (2012)). While low in percentages, therapies against these fusions have generated tremendous clinical impacts and are considered as recent milestones in cancer treatments. In particular, FDA granted accelerated approval to the first pan-cancer drug for the treatment of solid tumors, larotrectinib targeting NTRK fusions (Laetsch TW (2018)). Breast cancer accounts for 2.1 million new cancer cases world-wide annually. These results indicate the uses of HER2/HER3/SRC inhibitors to treat ESR1-CCDC170 positive patients. Based on the frequency observed in luminal B, endocrine resistant, and metastatic breast cancers, ESR1-CCDC170 can affect a patient population comparable to that of EML4-ALK positive lung cancer or NTRK positive adult cancer patients.

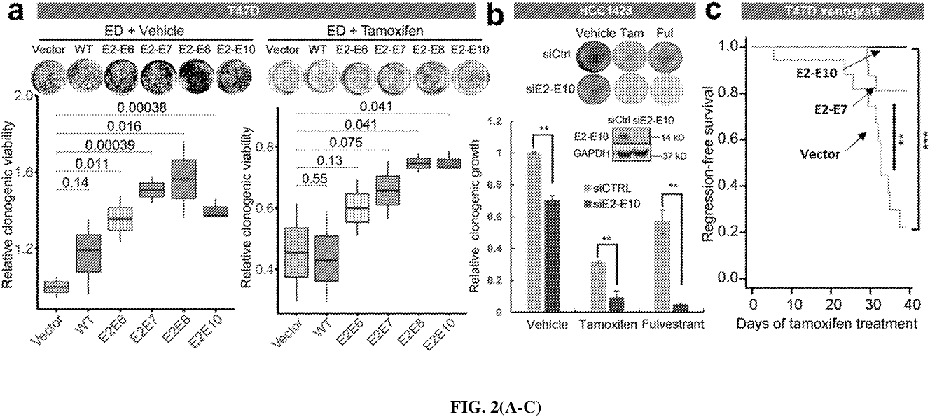

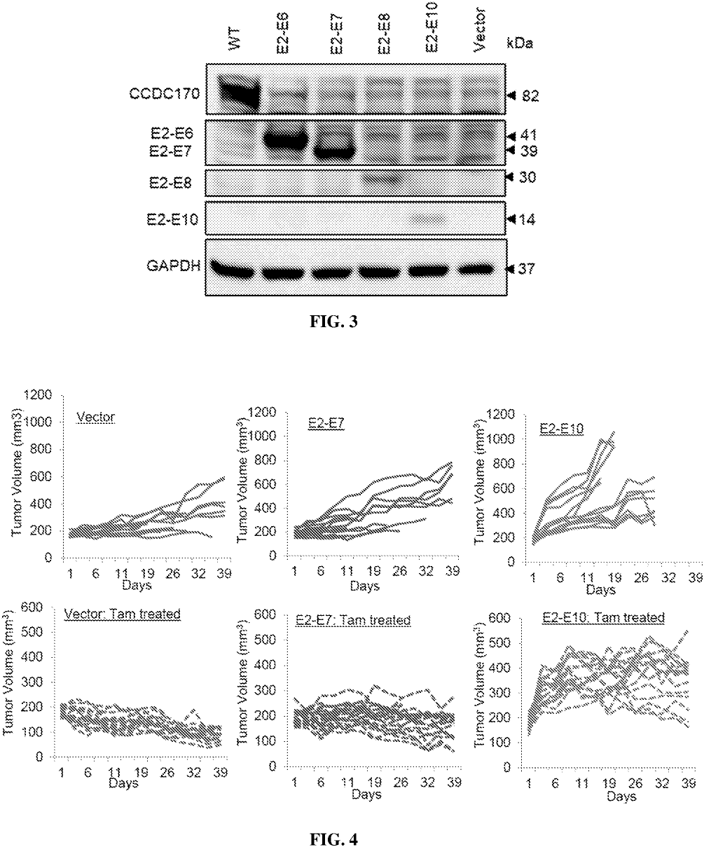

ESR1-CCDC170 fusions endow reduced endocrine sensitivity in vitro and in vivo. To explore their role in endocrine resistance, the consequences of ESR1-CCDC170 ectopic expression on the outcome of tamoxifen treatment in vitro and in vivo was assessed. First, the engineered T47D cells (FIG. 3) were treated with estrogen deprivation in the presence or absence of 0.5 µM 4-hydroxytamoxifen (4-OHT), the active metabolite of tamoxifen used for in vitro experiments. Clonogenic assays revealed that all ESR1-CCDC170 variants share the ability to promote some levels of reduced endocrine sensitivity, with the smaller-sized variants E2-E8 and E2-E10 conferring more potent effects (FIG. 2A). Next, the effect of depleting ESR1-CCDC170 on the endocrine sensitivity of HCC1428 was assessed, an ER+ breast cancer cell line harboring endogenous E2-E10 fusion. HCC1428 was derived from plural effusion of a 49 year old patient with ER+/HER2- metastatic luminal breast carcinoma after chemotherapy, who died 6 months later (Gazdar AF (1998)). Thus, this cell line is endocrine-therapy naive. A validated siRNA targeting the E2-E10 fusion junction that can specifically silence the E2-E10 fusion (Veeraraghavan J, (2014)) was used. The cells were then treated with tamoxifen or the second-line endocrine agent, fulvestrant, a more potent ER antagonist. Tamoxifen or fulvestrant alone reduced cell growth efficiently, whereas E2-E10 depletion led to more significant reduction of residue cancer cell colonies compared to the siRNA control (especially in fulvestrant-treated cells), indicating that silencing E2-E10 increases endocrine responsiveness of HCC1428 cells (FIG. 2B).

Next, the consequences of ESR1-CCDC170 ectopic expression on the outcome of tamoxifen treatment in vivo was assessed. T47D xenograft tumors expressing vector, E2-E7, or E2-E10 fusion variants were developed in female athymic nude mice. When the tumors reached 150-200 mm³, the mice were randomized into tamoxifen treatment (25 ug/kg) and untreated groups. When treated with tamoxifen, the vector group showed true tumor regression with 51% decrease in size, whereas the E2-E7 expressing tumors sustained steady tumor volumes with only 14% decrease in size. The E2-E10 expressing tumors continued to grow and then stabilized at higher tumor burdens, with almost two folds increase in tumor volumes (FIG. 4). Kaplan-Meier analysis revealed a significantly worse regression-free survival (time to tumor halving) for both E2-E7 and E2-E10-overexpressing tumors treated with tamoxifen compared to the vector control tumors (FIG. 2C). These data show that ESR1-CCDC170 variants render the T47D xenografts less sensitive to tamoxifen treatment in the in vivo context, with E2-E7 conferring partial response, and E2-E10 endowing stable/progressive disease.

It is notable that, ER-positive breast cancer patients are treated with endocrine therapy with a curable intent aimed to achieve long-term survival. Thus, cancer cell survival factor (i.e., E2-E7) is equally important as resistant factor (i.e., E2-E10). The relative tamoxifen-resistance of E2-E7 expressing xenograft tumors is comparable to that of the xenograft tumors expressing ESR1 mutations as previously reported (Toy W, (2017)). While mutations of ESR1 ligand binding domain constitute one of the most important driving mechanisms of acquired endocrine resistance (Ma CX (2015), Jeselsohn R (2015), Lei JT (2019)), the growth of the xenograft tumor models ectopically expressing ESR1 mutations can be effectively inhibited by endocrine treatment (Toy W, (2017)). The relative endocrine resistance of the xenograft tumors expressing ESR1 mutations was only evident when the different tumor models within the endocrine treated group are compared (Toy W, (2017)). Clinically the tumors expressing ESR1-CCDC170 fusions can respond to endocrine therapy initially but suffer from residue cancer cells that eventually can lead to metastatic relapse, a typical response pattern of endocrine-therapy naïve luminal B breast cancer.

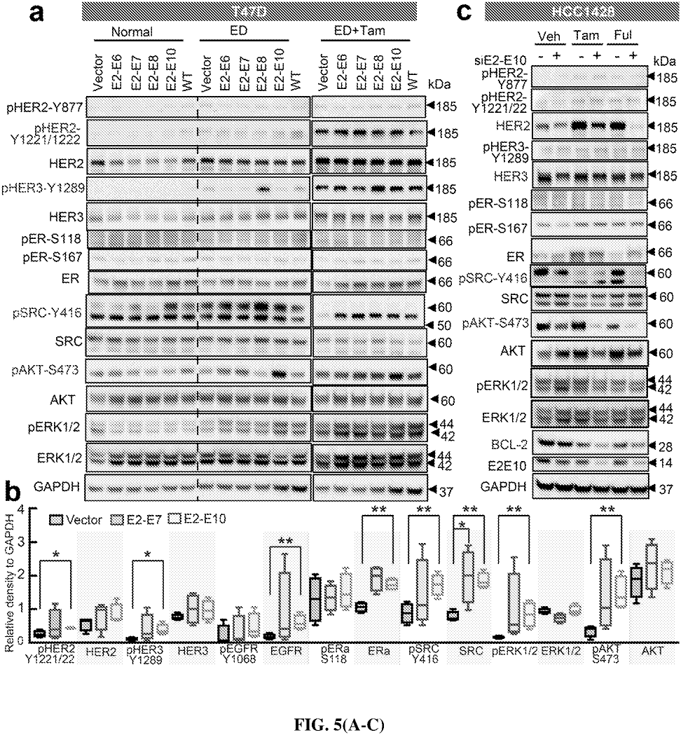

ESR1-CCDC170 fusions augment HER2/HER3/SRC/AKT pathway under endocrine treatment. Next, signaling alterations were examined in T47D cells ectopically expressing the ESR1-CCDC170 fusion variants or wtCCDC170. Under estrogen deprived condition, the phosphorylation levels of pHER3-Y1289 and pAKT-S473 were upregulated in only E2-E8 or E2-E10 variant, respectively (FIG. 5A). Whereas activation of pSRC-Y416 was induced by estrogen deprivation, and stronger activation was observed in T47D expressing ESR1-CCDC170 fusion variants compared to wtCCDC170 or vector controls. When cells are treated with tamoxifen, increased levels of HER2/HER3 were observed in all engineered T47D cells. This is consistent with the previous report that tamoxifen can stimulate HER2/HER3 expression (Moi LL (2012)). In addition, stronger activations of pHER2-Y1221/1222, pHER3-Y1289, pSRC-416 and pAKT-S473 were observed in the T47D cells expressing the fusion variants compared to wtCCDC170 or vector alone controls, indicating that ESR1-CCDC170 fusion variants overexpression enhances the activation of HER2/HER3 as well as its downstream SRC/PI3K/AKT pathways under tamoxifen treatment. Similar significant signaling alterations including increased phosphorylation of HER2, HER3, SRC, AKT, and ERK and increased EGFR, SRC and ER protein levels were observed in T47D xenograft tumors ectopically expressing E2-E10 variant, following treatment with tamoxifen (FIG. 5B), which are consistent with the tumor growth curve shown in FIG. 4, indicating that E2-E10 variants play an important role in activating of HER2/HER3 as well as the downstream SRC/PI3K/AKT pathways upon tamoxifen treatment..

Interestingly, estrogen deprivation induced activation of pSRC-Y416 and pAKT-S473, which was more potent in T47D expressing ESR1-CCDC170 variants compared to wtCCDC170 or vector control. In addition, the activation of pHER3-Y1289 was induced by estrogen deprivation in the E2-E8 expressing T47D cells (FIG. 5A). When cells were treated with tamoxifen, increased total HER2/HER3 levels were observed in all engineered T47D cells. This is consistent with the previous report that tamoxifen can stimulate HER2/HER3 expression (Moi LL, (2012)). In addition, stronger activations of pHER2-Y1221/1222, pHER3-Y1289, pSRC-416 and pAKT-S473 were observed in T47D cells expressing the fusion variants compared to wtCCDC170 or vector control, indicating that ESR1-CCDC170 fusions enhance the activation of HER2/HER3/SRC/AKT signaling under tamoxifen treatment. It is notable that the p-SRC-Y416 antibody detected two bands, and the upper band of the predicted size of 60kD was diminished following tamoxifen treatment. SRC can be cleaved by calpain (Hossain MI, (2013)), which generates a truncated Src of 52kD, the size of the lower band. Calpain may be activated by tamoxifen treatment (Storr SJ, (2015)), which cleaves SRC into a 52kD fragment. In the T47D xenograft tumors treated with tamoxifen, similar signaling alternations including increased activation of HER2, HER3, SRC, AKT, and ERK were observed in the E2-E10-expressing T47D tumors, and to a lesser degree in E2-E7-expressing tumors (FIG. 5B).

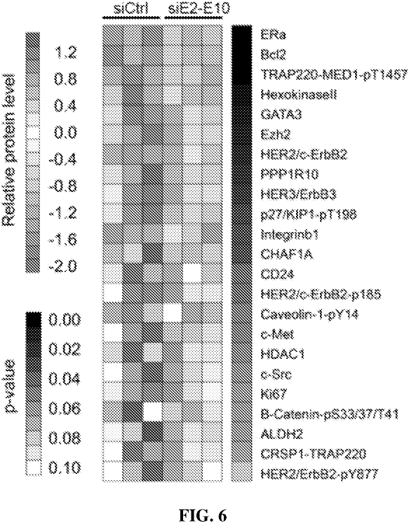

To gain insights into ESR1-CCDC170 driven mechanisms, reverse phase protein array (RPPA) analysis was performed in the HCC1428 cells with or without ESR1-CCDC170 silencing, using about 200 validated antibodies against an array of cancer related signaling molecules. ESR1-CCDC170 silencing leads to substantial repression of ERα, BCL2, HER3, c-SRC protein levels as well as total/phospho-HER2 (FIG. 6). Next, western blots were performed in E2-E10-silenced HCC1428 cells treated with vehicle, tamoxifen or fulvestrant which revealed repression of HER2, HER3, and BCL2 protein levels following E2-E10 depletion (FIG. 5C). Of note, HER2 protein level was upregulated by both tamoxifen and fulvestrant treatment, while depletion of E2-E10 counteracted this effect, especially under fulvestrant treatment. In addition, pSRC-Y416 and pAKT-S473 were repressed following E2-E10 silencing in HCC1428 cells, consistent with the observations from the T47D models.

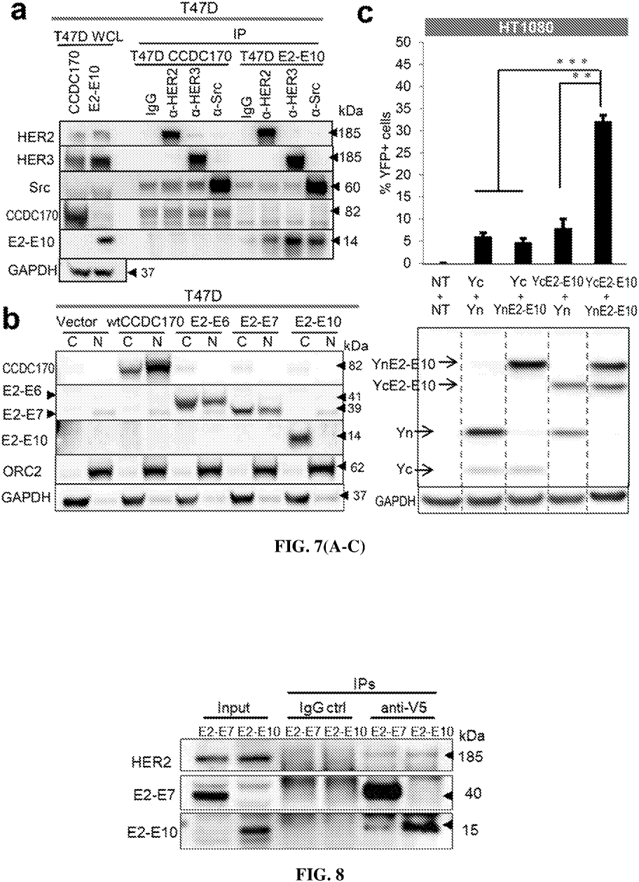

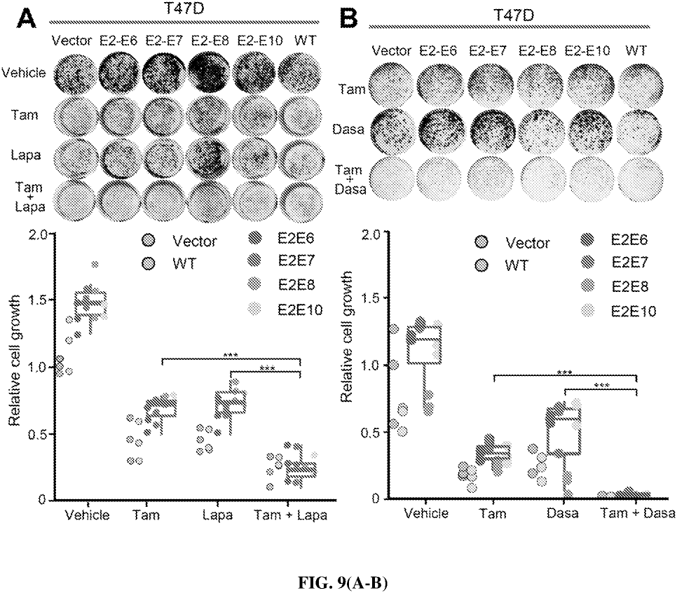

ESRI-CCDC170 localizes to cytoplasm, associates with HER2/HER3/SRC, and forms homodimers. HER2/HER3 heterodimer functions as an oncogenic unit and is known to engage SRC and PI3K/AKT pathway to drive breast cancer (Holbro T (2003); Yarden Y (2012)). Next experiment assessed whether ESR1-CCDC170 fusions modulate the HER2/HER3/SRC pathway by forming complex with them. To test this, immunoprecipitations were performed using HER2, HER3, or SRC antibodies on the T47D cells ectopically expressing E2-E10 or wtCCDC170, and performed western blots using anti-CCDC170 antibody. The ΔCCDC170 protein encoded by E2-E10, but not wtCCDC170, co-immunoprecipitated with endogenous HER2, HER3 and SRC (FIG. 7A & FIG. 8), indicating that E2-E10 may form complex with HER2, HER3 and SRC. This can be attributed to the conformational change or flexibility resulting from the truncation of CCDC170 (Ghadie MA (2017)). The interaction between HER2 and ΔCCDC170 were further verified on T47D cells ectopically expressing V5-E2-E7 or V5-E2-E10 via immunoprecipitation using V5 antibody and western blots using HER2 antibody. The experiment examined the subcellular localizations of ectopically expressed ESR1-CCDC170 proteins which revealed that the ΔCCDC170 proteins are more enriched in the cytoplasm, in contrast to the nuclear enriched wtCCDC170 (FIG. 7B). Among the fusion variants, E2-E10 is mostly localized to cytoplasm, in line with its stronger effect on endocrine resistance.

Wild type CCDC170 contains a structural maintenance of chromosomes (SMC) domain. The SMC proteins contains highly conserved ATP-binding cassette (ABC) that drives its dimerization (Hirano T (2006)). The ΔCCDC170 proteins encoded by the ESR1-CCDC170 fusions have different degrees of deletion of the N-terminal region of the SMC domain, but retain a putative high-affinity ATP-binding pocket at its C-terminus (FIG. 1C). The sequence of E2-E10 protein is SEQ ID NO: 34, IKTLEQTKAIEDLNKSRDQLEKMKEKAEKKLMSVKSELDTTEHEAKENKERARNMI EVVTSEMKTLKKSLEEAEKREKQLADFREVVSQMLGLNVTSLALPDYEIIKCLERLV HSHQHHFVTCACLKDVTTGQERHPQGHLQLLH (the putative ATP-binding cassette at the C-terminus of the E2-E10 protein is shown as the bolded, italicized amino acids; the coiled-coil domain is bolded amino acids). Therefore, it was investigated whether the N-terminal truncated CCDC170 proteins encoded by ESR1-CCDC170 can form dimers, which helps stabilize the HER2/HER3/SRC complex. To test this, Bimolecular Fluorescence Complementation (BiFC) assay was performed, which detects the proximity of two interacting proteins via reconstituted YFP fluorescence (Zheng ZY (2012)). Here the E2-E10 fusion variant was selected. It encodes the smallest ΔCCDC170 protein with most N-terminal truncation. The E2-E10 ORF was either fused to an N-terminal fragment of YFP (Yn) or to a C-terminal fragment of YFP (Yc) within the plasmid. The reconstituted YFP signal was only detectable in the cells co-transfected with Yn-E2-E10 and Yc-E2-E10 plasmid, indicating that E2-E10 forms homodimer (FIG. 7C).

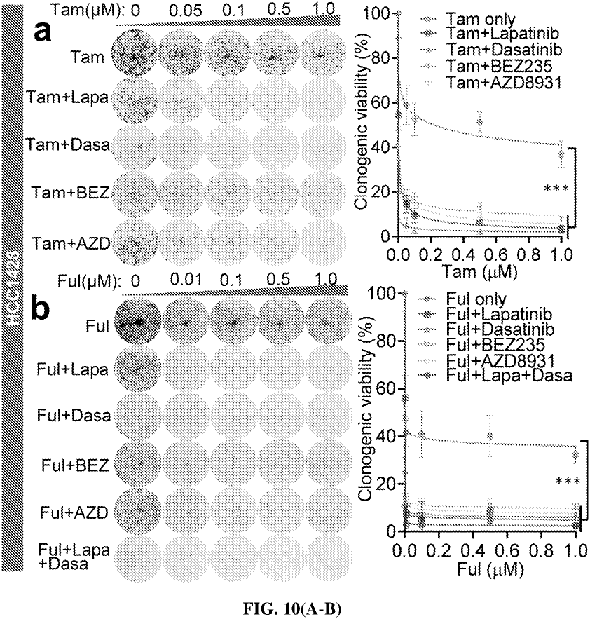

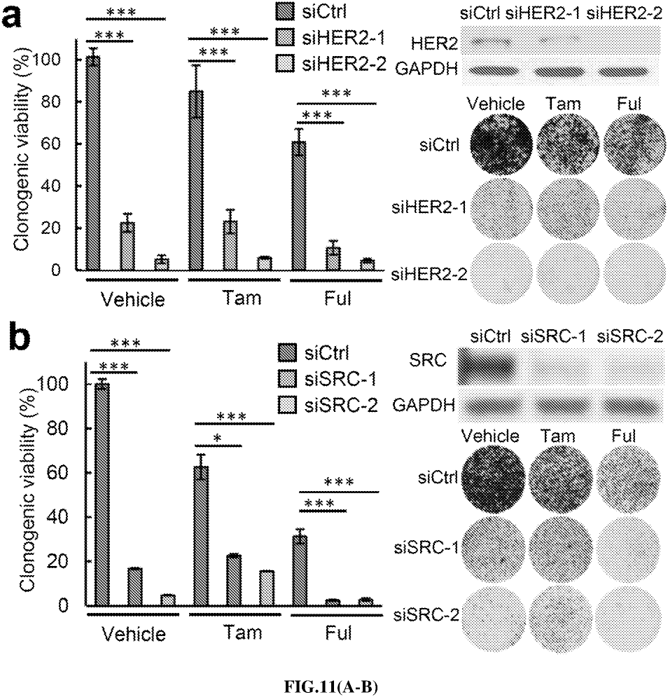

SRC and HER2 inhibitors increase endocrine sensitivity of luminal breast cancer cells expressing ESRI-CCDC170. Next, whether HER2/SRC inhibitors can revert the tamoxifen resistance driven by ESR1-CCDC170 was tested. Two drugs were selected for this test, lapatinib, a dual-specificity inhibitor targeting EGFR and HER-2 (Yarden Y, (2012)), and the SRC inhibitor dasatinib, both of which are FDA-approved (Schenone S (2010), Araujo J (2010)). The T47D cells ectopically expressing ESR1-CCDC170 variants were cultured in estrogen-deprived condition and treated either with tamoxifen alone or combined with lapatinib or dasatinib. Clonogenic assay results showed that the colony formations were significantly reduced when the cells were treated with tamoxifen plus lapatinib (FIG. 9A) or dasatinib (FIG. 9B) compared to tamoxifen monotreatment. Next, the HCC1428 cells were treated with lapatinib, dasatinib, BEZ235 (PI3K/mTOR inhibitor), or AZD8931 (EGFR/HER2/HER3 inhibitor), in combination with tamoxifen or fulvestrant (FIGS. 10A-10B). While all these inhibitors significantly inhibited cell viability compared to tamoxifen or fulvestrant treatment alone, lapatinib and dasatinib showed better therapeutic activities than BEZ235 or AZD8931, indicating the importance of directly targeting SRC/HER2 to block fusion-driven oncogenic signaling. In the presence of tamoxifen, dasatinib showed better activity than lapatinib (FIG. 10A). When combined with fulvestrant, lapatinib and dasatinib showed comparable therapeutic effects. Combining both lapatinib and dasatinib with fulvestrant resulted in additional therapeutic benefit which almost wiped out the cells (FIG. 10B). To verify the specific effect of HER2 or SRC inhibition in HCC1428 cells, we depleted HER2 or SRC using their specific siRNAs and treated the cells with tamoxifen or fulvestrant (FIGS. 11A-11B). The efficiency of these siRNAs was validated by western blots (Li L (2020)). Both HER2 and SRC silencing led to potent repression of cell viability especially when combined with fulvestrant treatment, similar to the effect of ESR1-CCDC170 silencing.

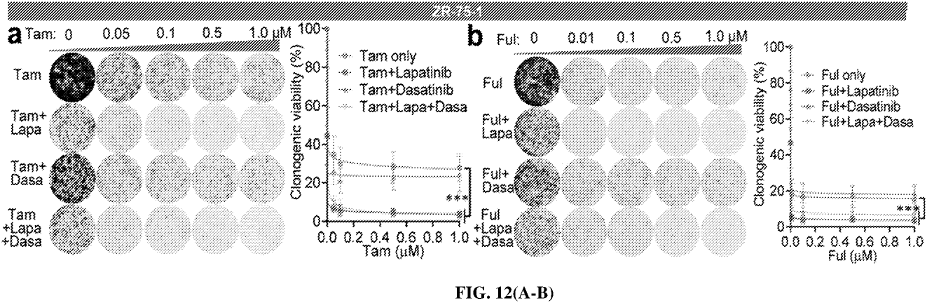

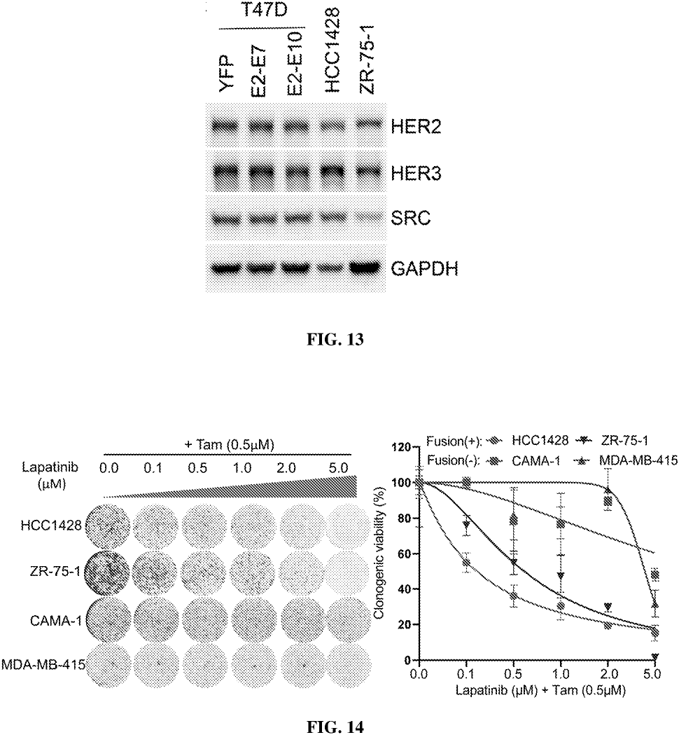

The therapeutic effect of lapatinib and dasatinib was then assessed in ZR-75-1 cells harboring the E2-E6 fusion (Veeraraghavan J, (2014)). ZR-75-1 is an ER+/HER2- endocrine therapy-naive cell line derived from ascitic effusion of a 63 year-old metastatic breast cancer patient who was subsequently treated with tamoxifen without apparent benefit (Engel LW, (1978)). This cell line was excluded from genetic silencing study as the fusion variant expressed in this cell line is not amendable to design siRNAs against its fusion junction due to their general toxicity to the cells. However, this cell line can be useful to provide additional insights into the therapeutic values of HER2/SRC inhibition in fusion positive cancer cells. The ZR-75-1 cells were treated with increasing doses of tamoxifen or fulvestrant, in combination with lapatinib, dasatinib, or both (FIGS. 12A-12B). ZR-75-1 cells responded to both tamoxifen and fulvestrant treatment, with fulvestrant showing more potent effect. Concomitant lapatinib treatment resulted in further decreased cell viability compared to endocrine treatment alone, whereas dasatinib did not show any significant therapeutic benefit. Western blots revealed that the expression of SRC in ZR-75-1 cells appears to be at very low level (FIG. 13). This shows that the treatment strategy for managing ESR1-CCDC170 positive breast tumors may depend on the context of SRC expression.

ESR1-CCDC170 positive luminal breast cancer cells show increased sensitivity to lapatinib in the context of endocrine therapy. To test the accountability of ESR1-CCDC170 on the Tamoxifen-sensitizing effects of lapatinib, the ESR1-CCDC170 positive cell lines HCC1428 (E2-E10 variant) and ZR-75-1 (E2-E6 variant), and fusion-negative cell lines MDA-MB-415 and CAMA-1 were treated with titrated dosages of Lapatinib in combination with 0.5 µM tamoxifen. All these cell lines are ER+/HER2- luminal breast cancer cell lines. The clonogenic viabilities following the respective treatments were assessed by clonogenic assays. The results showed that HCC1428 and ZR-75-1 cells exhibited substantially increased sensitivity to lapatinib than MDA-MB-415 and CAMA-1 cells in the context of tamoxifen treatment (FIG. 14). The IC50 for HCC1428 and ZR-75-1 cells are 0.15 and 0.6 µM, whereas the IC50 for MDA-MB-415 and CAMA-1 are 4.0 and 6.5 µM. The clinically relevant concentration for lapatinib is between 1.0-2.0 µM. This data further supports the accountability of ESR1-CCDC170 on the tam-sensitizing effect of lapatinib.

ESRI-CCDC170 proteins can facilitate HER2, HER3, SRC interactions, leading to uncontrolled activation of their downstream signaling. HER2 are amplified in approximately 15-20% of breast cancer, for which HER2-targeted therapy is the standard-of-care. While modest HER2 protein expression (IHC 1+ or 2+) can be detected in 60% of luminal breast cancer (Zhang H, (2018)), HER2 inhibitors are not indicated in these tumors. A recent clinical trial indicates that adding lapatinib to endocrine therapy in unselected advanced ER-positive breast cancer does not improve patient survival (Burstein HJ, (2014)). HER2/HER3 forms heterodimer when bind to HER3 ligand NRG½, which phosphorylates HER3 C-terminal tail thus activates its kinase activity (Lee-Hoeflich ST, (2008)). HER3 plays a central role in HER2 positive breast cancer and is the most potent activator of AKT/SRC (Lee-Hoeflich ST (2008), Lyu H (2018)). SRC is broadly overexpressed in luminal breast cancer (Anbalagan M (2012)) and can crosstalk with HER2 when facilitated by other molecules such as CDCP1, leading to phosphorylation of HER2 at the Y877 (Yarden Y (2012), Holbro T (2003), Alajati A (2015)). While SRC plays a key role in breast cancer bone metastasis and hormonal therapy resistance (Kennedy LC (2019), Vallabhaneni S (2010), Zhang XH (2009)), the clinical trials of SRC inhibitors in unselected metastatic luminal breast cancer patients have been disappointing (Kennedy LC, (2018)). SMC proteins are formed by two long coiled-coil domains connected by a non-helical sequence called “hinge”, which presumably corresponds to the low complexity region of CCDC170 (FIG. 1C). SMC proteins contain highly conserved ATP-binding cassette (ABC) that drives its dimerization, and fold back on themselves at the hinge region through antiparallel coiled-coil interactions (Hirano T, (2006)).

Based on the above, it is possible that the N-terminal truncations of CCDC170 that delete the CC1 and CC2 domains can expose the CC4 coiled-coil domain via reducing antiparallel coiled-coil interactions and thus alter protein interaction and localization properties. Through ATP-driven autonomous homodimerization, ESR1-CCDC170 proteins can facilitate interactions between HER2, HER3, or SRC, leading to uncontrolled activation of their downstream signaling (FIG. 1D). Therapeutic targeting the HER2/HER3/SRC complex can be an effective therapeutic strategy to treat ESR1-CCDC170 positive tumors. Since activated HER3 is known to dock SRC (Black LE, (2019)), and ESR1-CCDC170 appears to act in ZR-75-1 cells that lack SRC expression, ESR1-CCDC170 can facilitate HER2/HER3 heterodimerization, leading to recruitment of SRC to the activated HER3. This model in FIG. 1D explains the following facts: a) the fusion variants V2-5 leading to removal of CC½ domains have been associated with worse patient survival and reduced endocrine sensitivity in patients. b) Increased exposure of CC4 domain in fusion variants V4-5 confers more obvious endocrine resistance. c) Ectopic expression of ESR1-CCDC170 leads to activation of SRC/AKT, which are repressed when endogenous ESR1-CCDC170 is silenced. d)HER2/HER3 dimerization is known to prevent receptor internalization and lysosomal degradation (Bertelsen V, (2014)), which can explain the decreased HER2/HER3 protein levels following fusion silencing, an effect similar to Pertuzumab treatment (Hughes JB, (2012)).

Revealing an Achilles’ heel of the deadly forms of luminal breast cancers harboring ESRI-CCDC170 fusions. Endocrine resistant and metastatic tumors account for most of devastating deaths from luminal breast cancers. A recent study indicated that ESR1-CCDC170 significantly correlates with poor disease-free survival of breast cancer patients following surgery, accounting for 9% of distant relapse (AM Sieuwerts SV, (2019)). These patients’ lives could be saved if they can be appropriately treated and followed up after surgery. ESR1-CCDC170 engenders a special exploitable vulnerability in these deadly tumors by conferring sensitivity to HER2/HER3/SRC inhibitors. While HER2 and SRC have been implicated in endocrine resistance and metastasis, HER2/SRC inhibitors are not clinically indicated for ESR1-CCDC170 positive patients and their clinical trials in unselected luminal breast cancer patients have been disappointing. Thus, the finding shown herein can benefit a substantial population of ESR1-CCDC170 positive patients that otherwise can have a devastating clinical outcome.

A novel genotype-directed therapy for saving the lives and improving the life quality of ESRI-CCDC170+ patients. Luminal B and metastatic breast tumors are routinely treated with hormone therapy and chemotherapy that have life-threatening toxicity. This innovation can lead to a new paradigm to treat ESR1-CCDC170 positive patients with more potent therapeutic effect and much less toxicity to drastically improve their life quality. While HER2/HER3/SRC inhibitors are not novel, their indications in unselected luminal breast cancer patients are not supported by recent clinical trials that lack the guidance of predictive biomarkers: adding lapatinib to endocrine therapy in advanced ER-positive breast cancer does not improve patient survival (Burstein HJ, (2014)), and clinical trials of dasatinib in metastatic luminal breast cancers have been disappointing (Kennedy LC, (2019)). The data shown herein repurposes these drugs to treat genotypically selected luminal breast cancer patients based on ESR1-CCDC170 positivity.

### Example 2 Materials and Methods

Cell culture. ER+ breast cancer cell lines T47D cells and HCC1428 were purchased from American Type Culture Collection, cultured in RPMI-1640 (Corning) supplemented with 10% fetal bovine serum (Hyclone). For estrogen deprivation, cells were cultured in phenol red-free RPMI 1640 (Invitrogen) supplemented with 5% charcoaldextran- stripped serum (Sigma).

Engineering ectopic overexpression models. Lentiviral constructs and the lentivirus for ectopic expression of ESR1-CCDC170 fusion variants and wtCCDC170 were from our previous study (Veeraraghavan J (2014)). The T47D cells were infected by lentivirus containing these constructs in medium containing 4 µg/ml polybrene. Medium was replaced after overnight incubation. The cells with high GFP reporter expression were selected using flow cytometry two days later.

Antibodies and Reagents. Primary antibodies against pEGFR-Y1068 (D7A5), EGFR, pHER2Y877), pHER2-Y1221/1222 (6B12), pHER2- Y1248, HER2/ErbB2 (D8F12), pHER3-Y1289 (D1B5), HER3 (D22C5), pMet-Y1234/1235 (D26), Met (D1C2), pER-S118α (16J4), pERα-S167 (D1A3), ERα (D8H8), pAKT-S473 (D9E), AKT (C67E7), pERK (137F5), ERK (D13E1), pSrc-S416 (D49G4), Src (32G6), Integrin β1(D2E5) and Bcl-2 were purchased from Cell Signaling Technology. HER2/neu Ab-8 (Clone e2-4001) mouse monoclonal antibody used for IP is from Thermo. C6orf97 antibody is from Gene Tex. V5 antibody MA5-15253 is from Thermo. (Z)-4-Hydroxytamoxifen is from sigma. Fulvestrant (ICI-182780, ZD 9238), Lapatinib (GW572016), and Dasatinib were purchased from selleckchem, Drugs were resolved in DMSO, diluted in culture medium before use.

In vivo xenograft experiments. All animal experiments were performed in accordance with the institutional guidelines and regulations, and the animal protocol was approved by the BCM Institutional Animal Care and Use Committee (Approval # AN-6123). Briefly, 1×10⁷ T47D cells ectopically expressing the empty vector, or the ΔCCDC170 fusion variants E2-E7 and E2-E10 were resuspended in 20% Matrigel solution and injected bilaterally to 4-6 week-old female athymic nude mice (Harlan Sprague-Dawley) supplemented with 60-day-release 170-estradiol pellets. Xenograft tumors of the T47D models were successfully engrafted in 6-9 mice per group. Growth of the xenograft tumors was monitored twice per week and tumor volume was measured using the formula ½ (length × width²). When the tumors reached 200 mm³, mice with tumors expressing the empty vector, E2-E7, and E2-E10 variants were each randomized to +/- tamoxifen treatment. Tamoxifen (25 µg/Kg body weight) was injected subcutaneously for 5 days/week. Growth of the xenograft tumors was monitored twice per week until the end of the experiment.

siRNA and transfection. The E2-E10-specific siRNA (5′-CAUCACUGAGAUUAAAACU-3′) (SEQ ID NO: 29), ERα-specific siRNA (5′-AGGCUCAUUCCAGCCACAGTT-3′) (SEQ ID NO: 30), HER2-specific siRNA-1 (L-003126-00, ON-TARGETplus Human ERBB2 siRNA SMARTPool), HER2-specific siRNA-2 (5′-CACGUUUGAGUCCAUGCCCAA-3′) (SEQ ID NO: 31), Src-specific siRNA-1 (J-003175-15, 5′-CCAAGGGCCUCAACGUGAA-3′) (SEQ ID NO: 32), and Src-specific siRNA-2 (J-003175-16, 5′-GGGAGAACCUCUAGGCACA-3′) (SEQ ID NO: 33) and control siRNA (D-001810-10-50, ON-TARGETplus Non-targeting Pool) were purchased from Dharmacon. All siRNAs were transfected using Lipofectamine RNAi MAX Reagent (Invitrogen) according to manufacturer’s instructions..

Reverse phase protein array analysis. Reverse phase protein array assay was performed as described previously (Kim JA (2016)). Briefly, protein lysates were prepared from HCC1428 cells with control or E2-E10 siRNA knockdown using modified Tissue Protein Extraction Reagent (TPER) (Pierce) and a cocktail of protease and phosphatase inhibitors (Roche Life Science). The protein lysates were then diluted into 0.5 mg/ml of total protein in SDS sample buffer and denatured on the same day. For each spot on the array, the-background-subtracted foreground signal intensity was subtracted by the corresponding signal intensity of the negative control slide (omission of primary antibody) and then normalized to the corresponding signal intensity of total protein for that spot. The median of the triplicate experimental values (normalized signal intensity) is taken for each sample for subsequent statistical analysis.

Protein subcellular localization. Fractionation of the nuclear and cytoplasmic proteins from T47D cells ectopically overexpressing the empty vector, wtCCDC170 or the ΔCCDC170 fusion variants E2-E7 and E2-E10 was performed using the NE-PER Nuclear and Cytoplasmic Extraction Kit (Thermo) according to manufacturer’s instructions. The fractionated protein samples (30 µg protein) were then analyzed by western blot, as previously described (Veeraraghavan J (2014)).

Immunoprecipitation. For IP with HER2/HER3/Src antibody. As described previously (Yan K (2015)), cells were harvested and lysed in NETN- 400 buffer (50 mM Tris-HCl, pH 8.0, 400 mM NaCl, 1 mM EDTA, and 0.5% Nonidet P-40), for 25 min on ice. The samples were centrifuged at 13,000 rpm for 15 min, and the supernatants were diluted with the same buffer without NaCl (NETN-0) to obtain a final concentration of NaCl at 150 mM. The samples were incubated with the appropriate antibodies at 4° C. with rocking for 2 h. Protein G agarose was then added, and the incubation was continued for an additional 2 h. Beads were then washed three times using the NETN-150 buffer. The bound proteins were eluted with 100 mM glycine, pH 2.5, and then neutralized by adding ⅒ vol of 1 M Tris-Cl, pH 8.0. Eluted proteins were separated on 4-12% SDS-PAGE and blotted with the corresponding antibodies as indicated.

Clonogenic assay. For T47D, cells cultured in normal medium, RPMI1640 with phenol red and 10% FBS, were seeded at a density of 5000 cells per well in 24/48-well plate, 24 hours later normal medium was changed to ED medium, phenol red-free RPMI1640 supplemented with 5% charcoal-stripped serum (CSS), with or without indicated drugs, for 14 days. For HCC1428, cells were seeded at a density of 5000 cells per well in 24-well plate, added the indicated drugs 24 hours later, continue to culture for 18 days. Then the cells were fixed and stained by crystal violet, the colonies formed were calculated as intensity by using Image J with Colony Area Plug In.

Statistical analysis. The results of all in vitro experiments were compared by Student’s t-tests, and all data are shown as mean ± standard deviation. For the in vivo study, statistical comparison of tumor volumes was performed using one-way analysis of variance. Long-term outcomes were evaluated by survival analysis methods. ‘Events’ were defined to mimic clinically relevant outcomes; time to tumor regression (tumor-volume-halving) was analyzed using Kaplan-Meier survival curves and compared by the generalized Wilcoxon test.

### References

1. Ma CX, Reinert T, Chmielewska I, Ellis MJ. Mechanisms of aromatase inhibitor resistance. Nat Rev Cancer. 2015;15(5):261-75. Epub 2015/04/25. doi: 10.1038/nrc3920. PubMed PMID: 25907219.

2. Britton DJ, Hutcheson IR, Knowlden JM, Barrow D, Giles M, McClelland RA, Gee JM, Nicholson RI. Bidirectional cross talk between ERalpha and EGFR signalling pathways regulates tamoxifen-resistant growth. Breast Cancer Res Treat. 2006;96(2):131-46. doi: 10.1007/s10549-005-9070-2. PubMed PMID: 16261397.

3. deGraffenried LA, Friedrichs WE, Russell DH, Donzis EJ, Middleton AK, Silva JM, Roth RA, Hidalgo M. Inhibition of mTOR activity restores tamoxifen response in breast cancer cells with aberrant Akt Activity. Clin Cancer Res. 2004;10(23):8059-67. doi: 10.1158/1078-0432.CCR-04-0035. PubMed PMID: 15585641.

4. Drury SC et al., Changes in breast cancer biomarkers in the IGF1R/PI3K pathway in recurrent breast cancer after tamoxifen treatment. Endocr Relat Cancer. 2011;18(5):565-77. doi: 10.1530/ERC-10-0046. PubMed PMID: 21734071.

5. Hutcheson IR, Knowlden JM, Madden TA, Barrow D, Gee JM, Wakeling AE, Nicholson RI. Oestrogen receptor-mediated modulation of the EGFR/MAPK pathway in tamoxifen-resistant MCF-7 cells. Breast Cancer Res Treat. 2003;81(1):81-93. doi: 10.1023/A:1025484908380. PubMed PMID: 14531500.

6. Musgrove EA, Sutherland RL. Biological determinants of endocrine resistance in breast cancer. Nat Rev Cancer. 2009;9(9):631-43. doi: 10.1038/nrc2713. PubMed PMID: 19701242.

7. Osborne CK, Bardou V, Hopp TA, Chamness GC, Hilsenbeck SG, Fuqua SA, Wong J, Allred DC, Clark GM, Schiff R. Role of the estrogen receptor coactivator AIB1 (SRC-3) and HER-2/neu in tamoxifen resistance in breast cancer. J Natl Cancer Inst. 2003;95(5):353-61. PubMed PMID: 12618500.

8. Osborne CK, Schiff R. Mechanisms of endocrine resistance in breast cancer. Annu Rev Med. 2011;62:233-47. doi: 10.1146/annurev-med-070909-182917. PubMed PMID: 20887199; PMCID: 3656649.

9. Parisot JP, Hu XF, DeLuise M, Zalcberg JR. Altered expression of the IGF-1 receptor in a tamoxifen-resistant human breast cancer cell line. Br J Cancer. 1999;79(5-6):693-700. doi: 10.1038/sj.bjc.6690112. PubMed PMID: 10070856; PMCID: 2362670.

10. Zhou W, Slingerland JM. Links between oestrogen receptor activation and proteolysis: relevance to hormone-regulated cancer therapy. Nat Rev Cancer. 2014;14(1):26-38. PubMed PMID: 24505618.

11. Jeselsohn R, Buchwalter G, De Angelis C, Brown M, Schiff R. ESR1 mutations-a mechanism for acquired endocrine resistance in breast cancer. Nat Rev Clin Oncol. 2015;12(10):573-83. Epub 2015/07/01. doi: 10.1038/nrclinonc.2015.117. PubMed PMID: 26122181; PMCID: PMC4911210.

12. Hartmaier RJ et al., Recurrent hyperactive ESR1 fusion proteins in endocrine therapy-resistant breast cancer. Ann Oncol. 2018;29(4):872-80. Epub 2018/01/24. doi: 10.1093/annonc/mdy025. PubMed PMID: 29360925; PMCID: PMC5913625.

13. Lei JT, Gou X, Seker S, Ellis MJ. ESR1 alterations and metastasis in estrogen receptor positive breast cancer. J Cancer Metastasis Treat. 2019;5. Epub 2019/05/21. doi: 10.20517/2394-4722.2019.12. PubMed PMID: 31106278; PMCID: PMC6519472.

14. Giltnane JM et al., Genomic profiling of ER(+) breast cancers after short-term estrogen suppression reveals alterations associated with endocrine resistance. Sci Transl Med. 2017;9(402). Epub 2017/08/11. doi: 10.1126/scitranslmed.aai7993. PubMed PMID: 28794284; PMCID: PMC5723145.

15. Robinson DR et al., Activating ESR1 mutations in hormone-resistant metastatic breast cancer. Nat Genet. 2013;45(12):1446-51. Epub 2013/11/05. doi: 10.1038/ng.2823. PubMed PMID: 24185510; PMCID: PMC4009946.

16. Veeraraghavan J, Tan Y, Cao XX, Kim JA, Wang X, Chamness GC, Maiti SN, Cooper LJ, Edwards DP, Contreras A, Hilsenbeck SG, Chang EC, Schiff R, Wang XS. Recurrent ESR1-CCDC170 rearrangements in an aggressive subset of oestrogen receptor-positive breast cancers. Nat Commun. 2014;5:4577. doi: 10.1038/ncomms5577. PubMed PMID: 25099679; PMCID: 4130357.

17. Yersal O, Barutca S. Biological subtypes of breast cancer: Prognostic and therapeutic implications. World J Clin Oncol. 2014;5(3):412-24. Epub 2014/08/13. doi: 10.5306/wjco.v5.i3.412. PubMed PMID: 25114856; PMCID: PMC4127612.

18. Schalbetter SA, Goloborodko A, Fudenberg G, Belton JM, Miles C, Yu M, Dekker J, Mirny L, Baxter J. SMC complexes differentially compact mitotic chromosomes according to genomic context. Nat Cell Biol. 2017;19(9):1071-80. Epub 2017/08/22. doi: 10.1038/ncb3594. PubMed PMID: 28825700; PMCID: PMC5640152.

19. Laflamme G, Tremblay-Boudreault T, Roy MA, Andersen P, Bonneil E, Atchia K, Thibault P, D’Amours D, Kwok BH. Structural maintenance of chromosome (SMC) proteins link microtubule stability to genome integrity. J Biol Chem. 2014;289(40):27418-31. Epub 2014/08/20. doi: 10.1074/jbc.M114.569608. PubMed PMID: 25135640; PMCID: PMC4183782.

20. Dunning AM, et al., Breast cancer risk variants at 6q25 display different phenotype associations and regulate ESR1, RMND1 and CCDC170. Nat Genet. 2016;48(4):374-86. Epub 2016/03/02. doi: 10.1038/ng.3521. PubMed PMID: 26928228; PMCID: PMC4938803.

21. Hong Y, Chen XQ, Li JY, Liu C, Shen N, Zhu BB, Gong J, Chen W. Current evidence on the association between rs3757318 of C6orf97 and breast cancer risk: a meta-analysis. Asian Pac J Cancer Prev. 2014;15(19):8051-5. Epub 2014/10/24. doi: 10.7314/apjcp.2014.15.19.8051. PubMed PMID: 25338983.

22. Matissek KJ, et al., . Expressed Gene Fusions as Frequent Drivers of Poor Outcomes in Hormone Receptor-Positive Breast Cancer. Cancer Discov. 2018;8(3):336-53. Epub 2017/12/16. doi: 10.1158/2159-8290.CD-17-0535. PubMed PMID: 29242214.

23. Fimereli D, Fumagalli D, Brown D, Gacquer D, Rothe F, Salgado R, Larsimont D, Sotiriou C, Detours V. Genomic hotspots but few recurrent fusion genes in breast cancer. Genes Chromosomes Cancer. 2018;57(7):331-8. Epub 2018/02/13. doi: 10.1002/gcc.22533. PubMed PMID: 29436103.

24. Lei JT, et al., Functional Annotation of ESR1 Gene Fusions in Estrogen Receptor-Positive Breast Cancer. Cell Rep. 2018;24(6):1434-44 e7. Epub 2018/08/09. doi: 10.1016/j.celrep.2018.07.009. PubMed PMID: 30089255; PMCID: PMC6171747.

25. Yang SYC et al., Landscape of genomic alterations in high-grade serous ovarian cancer from exceptional long- and short-term survivors. Genome Med. 2018;10(1):81. Epub 2018/11/02. doi: 10.1186/s13073-018-0590-x. PubMed PMID: 30382883; PMCID: PMC6208125.

26. Robinson DR, et al., Integrative clinical genomics of metastatic cancer. Nature. 2017;548(7667):297-303. Epub 2017/08/08. doi: 10.1038/nature23306. PubMed PMID: 28783718; PMCID: PMC5995337.

27. Nikhil Wagle CP et al., Count me in: A patient-driven research initiative to accelerate cancer research. Journal of Clinical Oncology. 2018;36(15):DOI: 10.1200/JCO.2018.36.15_suppl.e13501

28. Zardavas D et al., The AURORA initiative for metastatic breast cancer. Br J Cancer. 2014;111(10):1881-7. Epub 2014/09/17. doi: 10.1038/bjc.2014.341. PubMed PMID: 25225904; PMCID: PMC4229627.

29. AM Sieuwerts SV, M Bos, S Sleijfer and JW Martens. Recurrent ESR1 fusions in primary tumors; a promising predictive factor for outcome to first-line endocrine therapy? Cancer Research. 2019(Abstract P5-11-02). Epub February doi: 10.1158/1538-7445.

30. Mitelman F, Johansson B, Mertens F. The impact of translocations and gene fusions on cancer causation. Nat Rev Cancer. 2007;7(4):233-45. PubMed PMID: 17361217.

31. Koivunen JP et al., EML4-ALK fusion gene and efficacy of an ALK kinase inhibitor in lung cancer. Clin Cancer Res. 2008;14(13):4275-83. PubMed PMID: 18594010.

32. Singh D et al., Transforming fusions of FGFR and TACC genes in human glioblastoma. Science. 2012;337(6099):1231-5. Epub 2012/07/28. doi: 10.1126/science.1220834. PubMed PMID: 22837387.

33. Laetsch TW, Hawkins DS. Larotrectinib for the treatment of TRK fusion solid tumors. Expert Rev Anticancer Ther. 2018:1-10. Epub 2018/10/24. doi: 10.1080/14737140.2019.1538796. PubMed PMID: 30350734.

34. Li L, Lin L, Veeraraghavan J, Hu Y, Wang X, Lee S, Tan Y, Schiff R, Wang XS. Therapeutic role of recurrent ESR1-CCDC170 gene fusions in breast cancer endocrine resistance. Breast Cancer Res. 2020;22(1):84. Epub 2020/08/11. doi: 10.1186/s13058-020-01325-3. PubMed PMID: 32771039; PMCID: PMC7414578.

35. Gazdar AF et al., Characterization of paired tumor and non-tumor cell lines established from patients with breast cancer. Int J Cancer. 1998;78(6):766-74. PubMed PMID: 9833771.

36. Toy W et al., Activating ESR1 Mutations Differentially Affect the Efficacy of ER Antagonists. Cancer Discov. 2017;7(3):277-87. Epub 2016/12/18. doi: 10.1158/2159-8290.CD-15-1523. PubMed PMID: 27986707; PMCID: PMC5340622.

37. Moi LL, Flageng MH, Gjerde J, Madsen A, Rost TH, Gudbrandsen OA, Lien EA, Mellgren G. Steroid receptor coactivators, HER-2 and HER-3 expression is stimulated by tamoxifen treatment in DMBA-induced breast cancer. BMC Cancer. 2012;12:247. Epub 2012/06/19. doi: 10.1186/1471-2407-12-247. PubMed PMID: 22703232; PMCID: PMC3420308.

38. Hossain MI et al., A truncated fragment of Src protein kinase generated by calpain-mediated cleavage is a mediator of neuronal death in excitotoxicity. J Biol Chem. 2013;288(14):9696-709. Epub 2013/02/13. doi: 10.1074/jbc.M112.419713. PubMed PMID: 23400779; PMCID: PMC3617272.

39. Storr SJ, Thompson N, Pu X, Zhang Y, Martin SG. Calpain in Breast Cancer: Role in Disease Progression and Treatment Response. Pathobiology. 2015;82(3-4):133-41. Epub 2015/09/04. doi: 10.1159/000430464. PubMed PMID: 26330354.

40. Zheng ZY et al., CHMP6 and VPS4A mediate the recycling of Ras to the plasma membrane to promote growth factor signaling. Oncogene. 2012;31(43):4630-8. doi: 10.1038/onc.2011.607. PubMed PMID: 22231449; PMCID: 3326214.

41. Zheng ZY, Chang EC. A bimolecular fluorescent complementation screen reveals complex roles of endosomes in Ras-mediated signaling. Methods in enzymology. 2014;535:25-38. doi: 10.1016/B978-0-12-397925-4.00002-X. PubMed PMID: 24377915.

42. Yarden Y, Pines G. The ERBB network: at last, cancer therapy meets systems biology. Nat Rev Cancer. 2012;12(8):553-63. Epub 2012/07/13. doi: 10.1038/nrc3309. PubMed PMID: 22785351.

43. Schenone S, Brullo C, Musumeci F, Botta M. Novel dual Src/Abl inhibitors for hematologic and solid malignancies. Expert opinion on investigational drugs. 2010; 19(8):931-45.

44. Araujo J, Logothetis C. Dasatinib: a potent SRC inhibitor in clinical development for the treatment of solid tumors. Cancer Treat Rev. 2010;36(6):492-500. Epub 2010/03/17. doi: 10.1016/j.ctrv.2010.02.015. PubMed PMID: 20226597; PMCID: PMC3940067.

45. Engel LW, Young NA, Tralka TS, Lippman ME, O’Brien SJ, Joyce MJ. Establishment and characterization of three new continuous cell lines derived from human breast carcinomas. Cancer Res. 1978;38(10):3352-64. Epub 1978/10/01. PubMed PMID: 688225.

46. Zhang H, Han M, Varma KR, Clark BZ, Bhargava R, Dabbs DJ. High Fidelity of Breast Biomarker Metrics: A 10-Year Experience in a Single, Large Academic Institution. Appl Immunohistochem Mol Morphol. 2018;26(10):697-700. Epub 2018/08/11. doi: 10.1097/PAI.0000000000000697. PubMed PMID: 30095467.

47. Burstein HJ, Cirrincione CT, Barry WT, Chew HK, Tolaney SM, Lake DE, Ma C, Blackwell KL, Winer EP, Hudis CA. Endocrine therapy with or without inhibition of epidermal growth factor receptor and human epidermal growth factor receptor 2: a randomized, double-blind, placebo-controlled phase III trial of fulvestrant with or without lapatinib for postmenopausal women with hormone receptor-positive advanced breast cancer-CALGB 40302 (Alliance). J Clin Oncol. 2014;32(35):3959-66. Epub 2014/10/29. doi: 10.1200/JCO.2014.56.7941. PubMed PMID: 25348000; PMCID: PMC4251959.

48. Lee-Hoeflich ST, Crocker L, Yao E, Pham T, Munroe X, Hoeflich KP, Sliwkowski MX, Stem HM. A central role for HER3 in HER2-amplified breast cancer: implications for targeted therapy. Cancer Res. 2008;68(14):5878-87. Epub 2008/07/18. doi: 10.1158/0008-5472.CAN-08-0380. PubMed PMID: 18632642.

49. Lyu H, Han A, Polsdofer E, Liu S, Liu B. Understanding the biology of HER3 receptor as a therapeutic target in human cancer. Acta Pharm Sin B. 2018;8(4):503-10. Epub 2018/08/16. doi: 10.1016/j.apsb.2018.05.010. PubMed PMID: 30109175; PMCID: PMC6090011.

50. Anbalagan M, Moroz K, Ali A, Carrier L, Glodowski S, Rowan BG. Subcellular localization of total and activated Src kinase in African American and Caucasian breast cancer. PLoS One. 2012;7(3):e33017. Epub 2012/03/30. doi: 10.1371/journal.pone.0033017. PubMed PMID: 22457730; PMCID: PMC3310861.

51. Holbro T, Beerli RR, Maurer F, Koziczak M, Barbas CF, 3rd, Hynes NE. The ErbB2/ErbB3 heterodimer functions as an oncogenic unit: ErbB2 requires ErbB3 to drive breast tumor cell proliferation. Proc Natl Acad Sci U S A. 2003;100(15):8933-8. Epub 2003/07/11. doi: 10.1073/pnas.1537685100. PubMed PMID: 12853564; PMCID: PMC166416.

52. Alajati A et al., Interaction of CDCP1 with HER2 enhances HER2-driven tumorigenesis and promotes trastuzumab resistance in breast cancer. Cell Rep. 2015;11(4):564-76. Epub 2015/04/22. doi: 10.1016/j.celrep.2015.03.044. PubMed PMID: 25892239.

53. Kennedy LC, Gadi V. Dasatinib in breast cancer: Src-ing for response in all the wrong kinases. Ann Transl Med. 2018;6(Suppl 1):S60. Epub 2019/01/08. doi: 10.21037/atm.2018.10.26. PubMed PMID: 30613635; PMCID: PMC6291560 Precision Medicine (ownership and consulting), Novartis (consulting), Pfizer (consulting), Daichii Sankyo (consulting), Seattle Genetics (consulting), and Genentech (research funding). LC Kennedy has no conflicts of interest to declare.

54. Vallabhaneni S, Nair BC, Cortez V, Challa R, Chakravarty D, Tekmal RR, Vadlamudi RK. Significance of ER-Src axis in hormonal therapy resistance. Breast Cancer Res Treat. 2011;130(2):377-85. Epub 2010/12/25. doi: 10.1007/s10549-010-1312-2. PubMed PMID: 21184269; PMCID: PMC3243930.

55. Zhang XH, Wang Q, Gerald W, Hudis CA, Norton L, Smid M, Foekens JA, Massague J. Latent bone metastasis in breast cancer tied to Src-dependent survival signals. Cancer Cell. 2009;16(1):67-78. Epub 2009/07/04. doi: 10.1016/j.ccr.2009.05.017. PubMed PMID: 19573813; PMCID: PMC2749247.

56. Hirano T. At the heart of the chromosome: SMC proteins in action. Nat Rev Mol Cell Biol. 2006;7(5):311-22. Epub 2006/04/25. doi: 10.1038/nrm1909. PubMed PMID: 16633335.

57. Black LE, Longo JF, Carroll SL. Mechanisms of Receptor Tyrosine-Protein Kinase ErbB-3 (ERBB3) Action in Human Neoplasia. Am J Pathol. 2019;189(10):1898-912. Epub 2019/07/29. doi: 10.1016/j.ajpath.2019.06.008. PubMed PMID: 31351986; PMCID: PMC6892224.

58. Bertelsen V, Stang E. The Mysterious Ways of ErbB2/HER2 Trafficking. Membranes (Basel). 2014;4(3):424-46. Epub 2014/08/08. doi: 10.3390/membranes4030424. ubMed PMID: 25102001; PMCID: PMC4194043.

9. Hughes JB, Rodland MS, Hasmann M, Madshus IH, Stang E. Pertuzumab Increases 17-AAG-Induced Degradation of ErbB2, and This Effect Is Further Increased by Combining Pertuzumab with Trastuzumab. Pharmaceuticals (Basel). 2012;5(7):674-89. Epub 2012/01/01. doi: 10.3390/ph5070674. PubMed PMID: 24281706; PMCID: PMC3763667.

60. Cancer Genome Atlas N. Comprehensive molecular portraits of human breast tumours. Nature 2012;490:61-70

61. Morrison G, Fu X, Shea M, Nanda S, Giuliano M, Wang T, et al. Therapeutic potential of the dual EGFR/HER2 inhibitor AZD8931 in circumventing endocrine resistance. Breast cancer research and treatment 2014;144:263-72

62. Ghadie MA, Lambourne L, Vidal M, Xia Y. Domain-based prediction of the human isoform interactome provides insights into the functional impact of alternative splicing. PLoS Comput Biol 2017;13:e1005717

63. Giuliano M, Trivedi MV, Schiff R. Bidirectional Crosstalk between the Estrogen Receptor and Human Epidermal Growth Factor Receptor 2 Signaling Pathways in Breast Cancer: Molecular Basis and Clinical Implications. Breast Care (Basel) 2013;8:256-62

64. Massarweh S, Osborne CK, Creighton CJ, Qin L, Tsimelzon A, Huang S, et al. Tamoxifen resistance in breast tumors is driven by growth factor receptor signaling with repression of classic estrogen receptor genomic function. Cancer Res 2008;68:826-33

65. Hiscox S, Jordan NJ, Morgan L, Green TP, Nicholson RI. Src kinase promotes adhesion-independent activation of FAK and enhances cellular migration in tamoxifen-resistant breast cancer cells. Clin Exp Metastasis 2007;24:157-67

66. Kim JA, Tan Y, Wang X, Cao X, Veeraraghavan J, Liang Y, et al. Comprehensive functional analysis of the tousled-like kinase 2 frequently amplified in aggressive luminal breast cancers. Nat Commun 2016;7:12991

67. Yan K, Li L, Wang X, Hong R, Zhang Y, Yang H, et al. The deubiquitinating enzyme complex BRISC is required for proper mitotic spindle assembly in mammalian cells. J Cell Biol 2015;210:209-24.

1. A method of diagnosing a subject with increased resistance to an estrogen receptor antagonist comprising:
a. obtaining a biological sample from the subject; and
b. detecting an ESR1/CCDC170 gene fusion in the sample, wherein the detection indicates the subject has increased resistance to the estrogen receptor antagonist and the subject is diagnosed with increased resistance to the estrogen receptor antagonist.

2. The method of claim 1, wherein the ESR1/CCDC170 gene fusion is selected from the group consisting of a E2-E2 fusion, a E2-E4 fusion, a E2-E5 fusion, a E2-E6 fusion, a E2-E7 fusion, a E2-E8 fusion, and a E2-E10 fusion.

3. The method of claim 2, wherein the E2-E2 fusion comprises SEQ ID NO: 35, the E2-E4 fusion comprises SEQ ID NO: 36, the E2-E5 fusion comprises SEQ ID NO: 37, the E2-E6 fusion comprises SEQ ID NO: 38, the E2-E7 fusion comprises SEQ ID NO: 39, the E2-E8 fusion comprises SEQ ID NO: 40, and the E2-E10 fusion comprises SEQ ID NO: 41.

4. The method of claim 3, wherein the detection comprises contacting the biological sample with a reaction mixture comprising a probe specific for a fusion point nucleotide sequence in at least one of SEQ ID NO: 35, SEQ ID NO: 36, SEQ ID NO: 37, SEQ ID NO: 38, SEQ ID NO:39, SEQ ID NO:40 and SEQ ID NO:41.

5. The method of claim 1, wherein the detection comprises contacting the biological sample with a reaction mixture comprising two primers, wherein the first primer is complementary to a ESR1 polynucleotide sequence and the second primer is complementary to a CCDC170 polynucleotide sequence, wherein the ESR1/CCDC170 gene fusion is detectable by the presence of an amplicon generated by the first primer and the second primer.

6. The method of claim 1, wherein the detection comprises contacting the biological sample with a reaction mixture comprising two probes, wherein the first probe is complementary to a ESR1 polynucleotide sequence and the second probe is complementary to a CCDC170 polynucleotide sequence, wherein hybridization of the two probes on a ESR1/CCDC170 gene fusion sequence provides a detectable signal, and the ESR1/CCDC170 gene fusion is detectable by the presence of the signal.

7. The method of claim 5, wherein a first of the one or more primers or probes is selected from the group consisting of SEQ ID NO: 42 and SEQ ID NO:44 and a second of the one or more primers or probes is selected from the group consisting of SEQ ID NO: 43 and SEQ ID NO: 45.

8. The method of claim 5, wherein the primers are SEQ ID NO: 42 and SEQ ID NO: 43.

9. The method of claim 5, wherein the primers are SEQ ID NO:44 and SEQ ID NO: 45.

10. The method of claim 1 wherein the subject has a cancer.

11. The method of claim 10, wherein the subject has a breast cancer.

12. The method of claim 11, wherein the subject has a luminal B or metastatic breast cancer.

13. The method of claim 1, wherein the detection of the ESR1/CCDC170 gene fusion indicates an increased resistance to one or more of tamoxifen, clomifene, raloxifene, exemestane, fulvestrant and letrozole.

14. The method of claim 1, further comprising administering to the subject a therapeutically effective amount of a HER inhibitor and/or a SRC inhibitor.

15. The method of claim 14, wherein the HER inhibitor is a HER2 inhibitor.

16. The method of claim 15, wherein the HER2 inhibitor is lapatinib.

17. The method of claim 13, wherein the SRC inhibitor is dasatinib.

18. The method of claim 1, further comprising administering to the subject an estrogen receptor antagonist.

19. A method of treating a cancer in a subject comprising:
a. detecting an ESR1/CCDC170 gene fusion in a sample obtained from the subject;
b. administering to the subject a therapeutically effective amount of a HER inhibitor and/or a SRC inhibitor.

20. The method of claim 19, further comprising administering to the subject an estrogen receptor antagonist.

21. The method of claim 20, wherein the estrogen receptor antagonist is selected from the group consisting of tamoxifen, clomifene, raloxifene, exemestane, fulvestrant and letrozole.

22. The method of claim 19, wherein the ESR1/CCDC170 gene fusion is selected from the group consisting of a E2-E2 fusion, a E2-E4 fusion, a E2-E5 fusion, a E2-E6 fusion, a E2-E7 fusion, a E2-E8 fusion, and a E2-E10 fusion.

23. The method of claim 22, wherein the E2-E2 fusion comprises SEQ ID NO: 35, the E2-E4 fusion comprises SEQ ID NO: 36, the E2-E5 fusion comprises SEQ ID NO: 37, the E2-E6 fusion comprises SEQ ID NO: 38, the E2-E7 fusion comprises SEQ ID NO: 39, the E2-E8 fusion comprises SEQ ID NO: 40, and the E2-E10 fusion comprises SEQ ID NO: 41.

24. The method of claim 19, wherein the subject has a breast cancer.

25. The method of claim 24, wherein the subject has a luminal B or metastatic breast cancer.

26. The method of claim 19, wherein the HER inhibitor is a HER2 inhibitor.

27. The method of claim 26, wherein the HER2 inhibitor is lapatinib.

28. The method of claim 19, wherein the SRC inhibitor is dasatinib.

29. A method of detecting an ESR1/CCDC170 gene fusion comprising:
a. obtaining a biological sample from a subject; and
b. detecting the fusion in the sample.

30. The method of claim 29, wherein the detection comprises contacting the biological sample with a reaction mixture comprising a probe specific for a fusion point nucleotide sequence in at least one of SEQ ID NO: 35, SEQ ID NO: 36, SEQ ID NO: 37, SEQ ID NO: 38, SEQ ID NO:39, SEQ ID NO:40 and SEQ ID NO:41.

31. The method of claim 30, wherein a detectable moiety is covalently bonded to the probe.

32. A kit comprising one or more probes, wherein each probe specifically hybridizes to a fusion point nucleotide sequence within SEQ ID NO: 35, SEQ ID NO: 36, SEQ ID NO: 37, SEQ ID NO: 38, SEQ ID NO:39, SEQ ID NO:40 or SEQ ID NO:41.

33. The kit of claim 32, wherein a detectable moiety is covalently bonded to the probe.


SEQUENCE LISTING (45 sequences)

SEQ ID NO: 1 - protein, 594 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 2 - DNA, 169 bp, Homo sapiens
atggtcataacagcctcctgtctaccgactcagaacggattttaccaaaactgaaaatgcaggctccatgctcagaagctctttaacaggctcgaaaggtccatgctcctttctcctgcccattctatagcataagaagacagtctctgagtgataatcttctcttcaa

SEQ ID NO: 3 - DNA, 131 bp, Homo sapiens
gaagaagaaaactaggaaggagtaagcacaaagatctcttcacattctccgggactgcggtaccaaatatcagcacagcacttcttgaaaaaggatgtagattttaatctgaactttgaaccatcactgag

SEQ ID NO: 4 - DNA, 1390 bp, Homo sapiens
ctagagcatgggtggccattgttgacctacaggaggcaccactgtcaccaacaaagggtaacagtctttcttttcaatatttatttatatccagtatttattttcaatactgactatggagagagctctcctgtgctcaaacactgcaatactgggggtctttcaaagcacaaaaacatatatttgcatgatggcatcattaacatttttatggctttctatttcttttttgtactggtctcaagagccactcataaatctctcagtaactgcatagtgtcccagggccagagaccggccactcctggcattgtgattagagtcatttaatatccaaggtggtgactaatgtctggcaacaaagcctccattgggtgtcatgtgtcctgggaccctgagcgtgggcactctaggagcacctcagtattgcgtgttagtactatggccgagagaatagttgagaaagtggtcaagaggtggatccatgtgaacgccactgggaaatgagagacctcgttcccaatcacggtcagtgcaactcgaaagcctaaaatcagtttaaaacaaaggtatctacctttatcttatgttcatatcctaggcttttaataatacgtatttttcacatgtttacagaaagcagtcaactgagctattcatggaaaggtttgtgggtttggttaacgaagtggaggagtattacatttcagctggaaacacatccctagaatgccaaaacatttattccaaagtctggtttcctggtgcaatcggaggcatggcaatgcctctgttcagagactgggggctagggccagtaaggcatttgatccacatgtatcccagaaggcttttattgttaaattatattctttcggaaaaaccacccatgtcctattttgtaaacttgatatccatacacttttgactggcattctattttagccgtaagactatgattcacagcaagcctgtttttcctcttgcttggggtggcagcagaaagcatagggtactttccagcctccaagggtaggggcaaaggggctggggtttctcctccccagtacagctttctctggctgtgccacactgctccctgtgagcagacagcaagtctcccctcactccccactgccattcatccagcgctgtgcagtagcccagctgcgtgtctgccgggaggggctgccaagtgccctgcctactggctgcttcccgaatccctgccattccacgcacaaacacatccacacactctctctgcctagttcacacactgagccactcgcacatgcgagcacattccttccttccttctcactctctcggcccttgacttctacaagcccatggaacatttctggaaagacgttcttgatccagcagg

SEQ ID NO: 5 - DNA, 314 bp, Homo sapiens
atgcaaccgcacaccccattctatctgccctatctcggttacagtgtagtcctccccagggtcatcctatgtacacactacgtatttctagccaacgaggagggggaatcaaacagaaagagagacaaacagagatatatcggagtctggcacggggcacataaggcagcacattagagaaagccggcccctggatccgtctttcgcgtttattttaagcccagtcttccctgggccacctttagcagatcctcgtgcgcccccgccccctggccgtgaaactcagcctctatccagcagcgacgacaagtaaa

SEQ ID NO: 6 - DNA, 686 bp, Homo sapiens
aggagctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacag

SEQ ID NO: 7 - DNA, 165 bp, Homo sapiens
gcttaaaaataatctcctgccagcccagtgacaagcctgtcccacccggggagaatgccccggagtggcgtgcgggtcagccagggtctgcgcctcgcagccactgtggaaggagcgcggccggtccaggacacaggagaccactttgtgacttcaatggcgaag

SEQ ID NO: 8 - DNA, 270 bp, Homo sapiens
aatggtgctgtcatgtggactgtcctcccgagtgtcccactggatgttcagagaatttatgtgaaggtcacgtcatttagcattgagatgctgtggttaccttcttccatttcttccataatatgcagccacatctatgtgtgaagaaatgtaatagataaaatttctctggacgcataataatgtgagaaagattgtcacatgtcccagcaaattgttattaatataaatttgttacttggcaagctgagattttgcaagatgttactc

SEQ ID NO: 9 - DNA, 204 bp, Homo sapiens
atgtagtttaataaagtaataatttcaagaaacgtgggcaaataagaaagagtatgactttcttacaacccgcttgtaagtgatgtggtggtggtaatgatccatgattttgatgatgacgatgatgatgaaaatgaagtttttgtctcagtttgggtaggtggtatttctggatgcctcctatggaccctggagatgttcatc

SEQ ID NO: 10 - DNA, 54 bp, Homo sapiens
ctggcactacttcctgggcagttttcttggctccttgacttgttgggcggcttg

SEQ ID NO: 11 - DNA, 191 bp, Homo sapiens
gccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaag

SEQ ID NO: 12 - protein, 715 aa, Homo sapiens
MSLDCTSHIALGAASPAPEETYDHLSEVPVTREQLNHYRNVAQNARSELAATLVKFECAQSELQDLRSKMLSKEVSCQELKAEMESYKENNARKSSLLTSLRDRVQELEEESAALSTSKIRTEITAHAAIKENQELKKKVVELNEKLQKCSKENEENKKQVSKNCRKHEEFLTQLRDCLDPDERNDKASDEDLILKLRDLRKENEFVKGQIVILEETINVHEMEAKASRETIMRLASEVNREQKKAASCTEEKEKLNQDLLSAVEAKEALEREVKIFQERLLAGQQVWDASKQEVSLLKKSSSELEKSLKASQDAVTTSQSQYFSFREKIAALLRGRLSMTGSTEDTILEKIREMDSREESRDRMVSQLEAQISELVEQLGKESGFHQKALQRAQKAENMLETLQGQLTHLEAELVSGGVLRDNLNFEKQKYLKFLDQLSQKMKLDQMAAELGFDMRLDVVLARTEQLVRLESNAVIENKTIAHNLQRKLKTQKERLESKELHMSLLRQKIAQLEEEKQARTALVVERDNAHLTIRNLQKKVERLQKELNTCRDLHTELKAKLADTNELKIKTLEQTKAIEDLNKSRDQLEKMKEKAEKKLMSVKSELDTTEHEAKENKERARNMIEVVTSEMKTLKKSLEEAEKREKQLADFREVVSQMLGLNVTSLALPDYEIIKCLERLVHSHQHHFVTCACLKDVTTGQERHPQGHLQLLH

SEQ ID NO: 13 - DNA, 169 bp, Homo sapiens
gcggtgtttacccgttgcccgaggagacacccgcgccacccgccggctcccggcgccgccgcttcctcagggccggttccgggtccgagcgcgcccccgggctcgggtcgtcatgagcctggactgcaccagccatatcgcgctgggtgccgcttcgccagcgcccgag

SEQ ID NO: 14 - DNA, 129 bp, Homo sapiens
gaaacttacgatcatctttcggaagtcccggtcacgcgggagcagttaaaccactatcggaatgtggctcaaaatgctcgaagtgaacttgcagcaactttggtcaaatttgaatgtgctcagtctgag

SEQ ID NO: 15 - DNA, 257 bp, Homo sapiens
cttcaagacctccgatccaagatgctttctaaagaagtctcctgtcaagaactgaaagctgaaatggagagctacaaggaaaacaatgccagaaaatcatctctccttacctctttgagagacagagttcaggaactagaagaagaatcagcagcactttccacttctaaaatcagaacagaaatcacagctcacgctgcaatcaaggagaaccaggaattaaagaagaaagttgtagagttaaatgaaaaattaca

SEQ ID NO: 16 - DNA, 145 bp, Homo sapiens
aaagtgttcaaaagaaaatgaggagaataagaaacaagtttcaaagaattgcaggaaacatgaggaatttctgactcaactgcgtgactgcttggatccagatgagaggaatgacaaggcatcagatgaagatttaattttaaag

SEQ ID NO: 17 - DNA, 186 bp, Homo sapiens
cttagagacctgcgcaaagaaaatgaattcgtgaaaggacaaattgttattcttgaagagactataaatgtccatgagatggaagcaaaagctagcagagaaacgatcatgaggctggcttcagaagtcaacagagagcagaaaaaagctgcctcctgtactgaagagaaagagaagctgaaccag

SEQ ID NO: 18 - DNA, 318 bp, Homo sapiens
gacctgctcagtgctgtagaagcaaaagaagctcttgaaagggaagttaagatcttccaagaaaggctgcttgctggccagcaggtctgggatgcctcaaagcaggaagtgagcctcctgaagaaaagctcttctgagttggagaagagtttgaaggccagtcaggatgcagtcacaacctcacaaagccagtacttctcatttagggagaaaatcgcagccctccttaggggcagattgagcatgactgggtccactgaggacaccattttggagaagattcgagaaatggacagccgggaagaaagcagggaccgg

SEQ ID NO: 19 - DNA, 201 bp, Homo sapiens
atggtctcccagcttgaagcccaaatatctgagcttgttgaacagttgggaaaggagtctgggtttcaccagaaagctctccagagggcccagaaagcagagaatatgttggagactcttcagggtcagctgacacacctggaggcagagctggtttctggaggtgttttgcgagacaacttgaattttgagaaacaaaaa

SEQ ID NO: 20 - DNA, 253 bp, Homo sapiens
gaaagaggaagagattctgtgagatcactcattaactttgactttcatgtcccatttttgtttctgttcttgggtttagtatcttaaatttctggatcagctttctcagaaaatgaagttggaccagatggctgccgaacttggctttgacatgcggctggacgtggttttagctcgaacagagcagctggttcgtcttgagagcaatgcagtcattgagaacaagaccattgcccacaatttgcagagaaag

SEQ ID NO: 21 - DNA, 243 bp, Homo sapiens
ctaaagacacagaaagagagactggagagcaaagaattacacatgagcctcctccggcagaaaatagcccagctggaggaggagaagcaggcacgcacggccttggtggttgagagggacaacgcgcatcttaccatcaggaacttgcagaagaaggtggagaggctgcagaaagagctgaacacgtgtcgagacttgcacaccgagctcaaagccaaactggccgacaccaatgaactgaag

SEQ ID NO: 22 - DNA, 237 bp, Homo sapiens
attaaaactttggaacagactaaagccattgaagatctaaacaaatccagagaccaactggagaagatgaaggagaaagctgagaaaaagctcatgtctgtcaagtcagaactggataccacagaacatgaggctaaggagaataaagaaagggccagaaacatgatagaagtggtaaccagtgaaatgaagacactaaaaaaatctctggaagaagcagaaaagagagaaaagcag

SEQ ID NO: 23 - DNA, 1627 bp, Homo sapiens
aggagaaaatgaaaaatatacaaccaaccgttcgtgagtcatcaaaaagtcaaagtcagcctggccaacatggcaaaactccgtctctgcaaaaaatacaaaaatgagcccggtatgttggcatatgcctgtaatcccagctactcgagaggctgagacacgaaaattgcttgaacctgggaggcggaggttgcaatgagccgagatcgcgctactgcactccagcctgggcaacagagagagaccttgtctcaaaaaacaacaacaacaaaaagtcaaagtcataataagcaaattattggcttctttcttctagactaaaagaaattaaagagatgaaacaatcaattgcaagggtcaaaactagattggatcttggtttgaatgaaaaaaagcataaaatattcttgcaataattgtaaaaatttgaatgtggactaagtcctagattatattaaaatatttttaattttttaagcttgacaaatgcactgattgttatactttaaataactaaaaatctgagaatccacagtgctacagacaataagtgataaaatgggaaaaaatagtgaagatcttcttttaggaaaactaccaataaatgtagaatggatgattccaaaaaaaaaaaaaaaaagaagaaagagagaaagaaagaaacagaaaaacctaaaagaaagcgaatgaatttgacacctgttggttggatgacagaccacacagagggagagtgaccaggctttgcttgtggagcgggcatccctgcctagctatctccttcatgtgcaaggacagtataagtagtattttgcatgtgagtaaacacacctgcgcagccctggctgaggtcactaggctggccagaggcaggacaggcagtcttgaatttcctctaggggaggcctgcagttttccccaccaaccccttttcacatgctccaaagatgtagtagtgtctgctgttttggatcgaaaatcaccttgagtggaggaagtgacttcactgggtctctggaggctctcggagcttgagtggctctgccccaccctgaatcatgcacccataaatgcagttatgggtggatgtgggtgtatgagagtggagattgtttttgtacattctctttgttttcattttccaaatataatctttaataaacttgatttagtttcttgggaaatttattttctacgctattcaaagatattttaaataatattttaaaaacatgaaatttttttttcatttttgatttgatatcattaattttatatcaggattcatgtaagagactaaaaatgatccatagtggttagtactgtaatttctgtttattacatgaaattacattccatttatccaaatatatgtttatataatatttccactgtgtgactctagtgatctttaacatacacagaatgatctacagtgatctttaacatactcagaaatatgaaaaatgtttgaatatgatctttagggacttctaatgaaaagggtatatgaaatgggaacaataaattctgtacatgtatacagtccatatacacattaagtgtttgtcatttggacaaattgaaaacttttaataaagccattttcttctttgttaaaatgcca

SEQ ID NO: 24 - protein, 530 aa, Homo sapiens
MDIKNSPSSLNSPSSYNCSQSILPLEHGSIYIPSSYVDSHHEYPAMTFYSPAVMNYSIPSNVTNLEGGPGRQTTSPNVLWPTPGHLSPLVVHRQLSHLYAEPQKSPWCEARSLEHTLPVNRETLKRKVSGNRCASPVTGPGSKRDAHFCAVCSDYASGYHYGVWSCEGCKAFFKRSIQGHNDYICPATNQCTIDKNRRKSCQACRLRKCYEVGMVKCGSRRERCGYRLVRRQRSADEQLHCAGKAKRSGGHAPRVRELLLDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGISSQQQSMRLANLLMLLSHVRHASNKGMEHLLNMKCKNVVPVYDLLLEMLNAHVLRGCKSSITGSECSPAEDSKSKEGSQNPQSQ

SEQ ID NO: 25 - protein, 933 aa, Homo sapiens
MTELKAKGPRAPHVAGGPPSPEVGSPLLCRPAAGPFPGSQTSDTLPEVSAIPISLDGLLFPRPCQGQDPSDEKTQDQQSLSDVEGAYSRAEATRGAGGSSSSPPEKDSGLLDSVLDTLLAPSGPGQSQPSPPACEVTSSWCLFGPELPEDPPAAPATQRVLSPLMSRSGCKVGDSSGTAAAHKVLPRGLSPARQLLLPASESPHWSGAPVKPSPQAAAVEVEEEDGSESEESAGPLLKGKPRALGGAAAGGGAAAVPPGAAAGGVALVPKEDSRFSAPRVALVEQDAPMAPGRSPLATTVMDFIHVPILPLNHALLAARTRQLLEDESYDGGAGAASAFAPPRSSPCASSTPVAVGDFPDCAYPPDAEPKDDAYPLYSDFQPPALKIKEEEEGAEASARSPRSYLVAGANPAAFPDFPLGPPPPLPPRATPSRPGEAAVTAAPASASVSSASSSGSTLECILYKAEGAPPQQGPFAPPPCKAPGASGCLLPRDGLPSTSASAAAAGAAPALYPALGLNGLPQLGYQAAVLKEGLPQVYPPYLNYLRPDSEASQSPQYSFESLPQKICLICGDEASGCHYGVLTCGSCKVFFKRAMEGQHNYLCAGRNDCIVDKIRRKNCPACRLRKCCQAGMVLGGRKFKKFNKVRVVRALDAVALPQPVGVPNESQALSQRFTFSPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAAQLPKILAGMVKPLLFHKK

SEQ ID NO: 26 - protein, 1255 aa, Homo sapiens [sequence omitted]

SEQ ID NO: 27 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 28 - protein, 536 aa, Homo sapiens
MGSNKSKPKDASQRRRSLEPAENVHGAGGGAFPASQTPSKPASADGHRGPSAAFAPAAAEPKLFGGFNSSDTVTSPQRAGPLAGGVTTFVALYDYESRTETDLSFKKGERLQIVNNTEGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 29 - RNA, 19 nt, Artificial Sequence
  Synthetic construct
caucacugagauuaaaacu

SEQ ID NO: 30 - DNA, 21 bp, Artificial Sequence
  Synthetic construct
aggcucauuccagccacagtt

SEQ ID NO: 31 - RNA, 21 nt, Artificial Sequence
  Synthetic construct
cacguuugaguccaugcccaa

SEQ ID NO: 32 - RNA, 19 nt, Artificial Sequence
  Synthetic construct
ccaagggccucaacgugaa

SEQ ID NO: 33 - RNA, 19 nt, Artificial Sequence
  Synthetic construct
gggagaaccucuaggcaca

SEQ ID NO: 34 - protein, 145 aa, Homo sapiens
IKTLEQTKAIEDLNKSRDQLEKMKEKAEKKLMSVKSELDTTEHEAKENKERARNMIEVVTSEMKTLKKSLEEAEKREKQLADFREVVSQMLGLNVTSLALPDYEIIKCLERLVHSHQHHFVTCACLKDVTTGQERHPQGHLQLLH

SEQ ID NO: 35 - DNA, 5437 bp, Homo sapiens
atggtcataacagcctcctgtctaccgactcagaacggattttaccaaaactgaaaatgcaggctccatgctcagaagctctttaacaggctcgaaaggtccatgctcctttctcctgcccattctatagcataagaagacagtctctgagtgataatcttctcttcaagaagaagaaaactaggaaggagtaagcacaaagatctcttcacattctccgggactgcggtaccaaatatcagcacagcacttcttgaaaaaggatgtagattttaatctgaactttgaaccatcactgaggaaacttacgatcatctttcggaagtcccggtcacgcgggagcagttaaaccactatcggaatgtggctcaaaatgctcgaagtgaacttgcagcaactttggtcaaatttgaatgtgctcagtctgagcttcaagacctccgatccaagatgctttctaaagaagtctcctgtcaagaactgaaagctgaaatggagagctacaaggaaaacaatgccagaaaatcatctctccttacctctttgagagacagagttcaggaactagaagaagaatcagcagcactttccacttctaaaatcagaacagaaatcacagctcacgctgcaatcaaggagaaccaggaattaaagaagaaagttgtagagttaaatgaaaaattacaaaagtgttcaaaagaaaatgaggagaataagaaacaagtttcaaagaattgcaggaaacatgaggaatttctgactcaactgcgtgactgcttggatccagatgagaggaatgacaaggcatcagatgaagatttaattttaaagcttagagacctgcgcaaagaaaatgaattcgtgaaaggacaaattgttattcttgaagagactataaatgtccatgagatggaagcaaaagctagcagagaaacgatcatgaggctggcttcagaagtcaacagagagcagaaaaaagctgcctcctgtactgaagagaaagagaagctgaaccaggacctgctcagtgctgtagaagcaaaagaagctcttgaaagggaagttaagatcttccaagaaaggctgcttgctggccagcaggtctgggatgcctcaaagcaggaagtgagcctcctgaagaaaagctcttctgagttggagaagagtttgaaggccagtcaggatgcagtcacaacctcacaaagccagtacttctcatttagggagaaaatcgcagccctccttaggggcagattgagcatgactgggtccactgaggacaccattttggagaagattcgagaaatggacagccgggaagaaagcagggaccggatggtctcccagcttgaagcccaaatatctgagcttgttgaacagttgggaaaggagtctgggtttcaccagaaagctctccagagggcccagaaagcagagaatatgttggagactcttcagggtcagctgacacacctggaggcagagctggtttctggaggtgttttgcgagacaacttgaattttgagaaacaaaaatatcttaaatttctggatcagctttctcagaaaatgaagttggaccagatggctgccgaacttggctttgacatgcggctggacgtggttttagctcgaacagagcagctggttcgtcttgagagcaatgcagtcattgagaacaagaccattgcccacaatttgcagagaaagctaaagacacagaaagagagactggagagcaaagaattacacatgagcctcctccggcagaaaatagcccagctggaggaggagaagcaggcacgcacggccttggtggttgagagggacaacgcgcatcttaccatcaggaacttgcagaagaaggtggagaggctgcagaaagagctgaacacgtgtcgagacttgcacaccgagctcaaagccaaactggccgacaccaatgaactgaagattaaaactttggaacagactaaagccattgaagatctaaacaaatccagagaccaactggagaagatgaaggagaaagctgagaaaaagctcatgtctgtcaagtcagaactggataccacagaacatgaggctaaggagaataaagaaagggccagaaacatgatagaagtggtaaccagtgaaatgaagacactaaaaaaatctctggaagaagcagaaaagagagaaaagcagctggcagacttcagggaggtggtgtcgcagatgctaggcttgaacgtgaccagccttgctcttcctgattatgaaatcatcaagtgtcttgaaagattggtccattcacatcagcatcactttgttacctgtgcctgcctcaaagatgtgactactgggcaagagaggcacccacaaggccatttacagcttcttcattgaacactgtatctcttgagagaggtggccataagacatggcacacaattcccaatttcacaaattcctcatgtctttgagatttgatcagtttgtgaatattttatgctttgatgatatagtgagaatgcatcacttgcaaaaacgatctcaaaagtgtcagccttagataaacgttcagcattaaaaacgcctattatttcatttactagcattttaggatccagaagaattccaccagattgcatgagttagattgggaaatgggagtgggagataatattgggaggtatctattttaagtcaggggctttactagccgatttagttctcacaataaccatgtggagaagctgtgacatttttaatttacaacctttctggggctcagacataaagttacctatccaaggttgcagttgggtagtggtgggaccaggatggacaactcattggccctgcctcaaaagccatacctcttctcctgctatgcagaatctgtttctcctgaatctctgtgatgctggtgggaattgtttgcatagaggaaggacaataaccctgccatcgtgagttaatgtccgggctggtcacagtggttcatgcctgtaatcccagcactttgggagtccaaggcaggcatatcatttgaggtcaggagtttaagaccagcctggctaacatagtgagaccctgtttctactaaaaatacaaaaataagccaggtgtggtggtgcatgactgtaatcccaactactcagcaggagaagcacttgaacccaggagacggaggcggcagtgagccaagattgtgccactgcactccagcctgggcgacagagtgaaactacatctcagaaaaaaaaaaaaaaaaaaaaaaaaaaagttaatgtccaaaaatgacagatttacaagtgtaagctatatgatttcttcaaaaagcaaaagcaatatacctaattcatttggatcaaacttacataggtctcaggtcctgtaagaaacttgcctgttctaactgttgctaccagattatatctggtggtaattgttaatgtttcagcagggctggtctcagtcctttaaaatggaaagctttatttggagcccaaccctataagatgaagaaatcctatatagtcttattcaccaatatatccaatacacccacagcaatggtacctttttaagatcaggattttattatgaattcctgtcactttctgttttccatttaaatttctattttacaaatttttcagggaatcatattcttaacttcactgagaaaaatgtgttactcttttggacaatttatcttatttctatcatataagatgtatttttttattgtccttaaaagaagctctagcatgaaattaaaggaaagggaaagaaatagatctggtgcacccgaacattaggagaaaatgaaaaatatacaaccaaccgttcgtgagtcatcaaaaagtcaaagtcagcctggccaacatggcaaaactccgtctctgcaaaaaatacaaaaatgagcccggtatgttggcatatgcctgtaatcccagctactcgagaggctgagacacgaaaattgcttgaacctgggaggcggaggttgcaatgagccgagatcgcgctactgcactccagcctgggcaacagagagagaccttgtctcaaaaaacaacaacaacaaaaagtcaaagtcataataagcaaattattggcttctttcttctagactaaaagaaattaaagagatgaaacaatcaattgcaagggtcaaaactagattggatcttggtttgaatgaaaaaaagcataaaatattcttgcaataattgtaaaaatttgaatgtggactaagtcctagattatattaaaatatttttaattttttaagcttgacaaatgcactgattgttatactttaaataactaaaaatctgagaatccacagtgctacagacaataagtgataaaatgggaaaaaatagtgaagatcttcttttaggaaaactaccaataaatgtagaatggatgattccaaaaaaaaaaaaaaaaagaagaaagagagaaagaaagaaacagaaaaacctaaaagaaagcgaatgaatttgacacctgttggttggatgacagaccacacagagggagagtgaccaggctttgcttgtggagcgggcatccctgcctagctatctccttcatgtgcaaggacagtataagtagtattttgcatgtgagtaaacacacctgcgcagccctggctgaggtcactaggctggccagaggcaggacaggcagtcttgaatttcctctaggggaggcctgcagttttccccaccaaccccttttcacatgctccaaagatgtagtagtgtctgctgttttggatcgaaaatcaccttgagtggaggaagtgacttcactgggtctctggaggctctcggagcttgagtggctctgccccaccctgaatcatgcacccataaatgcagttatgggtggatgtgggtgtatgagagtggagattgtttttgtacattctctttgttttcattttccaaatataatctttaataaacttgatttagtttcttgggaaatttattttctacgctattcaaagatattttaaataatattttaaaaacatgaaatttttttttcatttttgatttgatatcattaattttatatcaggattcatgtaagagactaaaaatgatccatagtggttagtactgtaatttctgtttattacatgaaattacattccatttatccaaatatatgtttatataatatttccactgtgtgactctagtgatctttaacatacacagaatgatctacagtgatctttaacatactcagaaatatgaaaaatgtttgaatatgatctttagggacttctaatgaaaagggtatatgaaatgggaacaataaattctgtacatgtatacagtccatatacacattaagtgtttgtcatttggacaaattgaaaacttttaataaagccattttcttctttgttaaaatgcca

SEQ ID NO: 36 - DNA, 5051 bp, Homo sapiens
atggtcataacagcctcctgtctaccgactcagaacggattttaccaaaactgaaaatgcaggctccatgctcagaagctctttaacaggctcgaaaggtccatgctcctttctcctgcccattctatagcataagaagacagtctctgagtgataatcttctcttcaagaagaagaaaactaggaaggagtaagcacaaagatctcttcacattctccgggactgcggtaccaaatatcagcacagcacttcttgaaaaaggatgtagattttaatctgaactttgaaccatcactgagaaagtgttcaaaagaaaatgaggagaataagaaacaagtttcaaagaattgcaggaaacatgaggaatttctgactcaactgcgtgactgcttggatccagatgagaggaatgacaaggcatcagatgaagatttaattttaaagcttagagacctgcgcaaagaaaatgaattcgtgaaaggacaaattgttattcttgaagagactataaatgtccatgagatggaagcaaaagctagcagagaaacgatcatgaggctggcttcagaagtcaacagagagcagaaaaaagctgcctcctgtactgaagagaaagagaagctgaaccaggacctgctcagtgctgtagaagcaaaagaagctcttgaaagggaagttaagatcttccaagaaaggctgcttgctggccagcaggtctgggatgcctcaaagcaggaagtgagcctcctgaagaaaagctcttctgagttggagaagagtttgaaggccagtcaggatgcagtcacaacctcacaaagccagtacttctcatttagggagaaaatcgcagccctccttaggggcagattgagcatgactgggtccactgaggacaccattttggagaagattcgagaaatggacagccgggaagaaagcagggaccggatggtctcccagcttgaagcccaaatatctgagcttgttgaacagttgggaaaggagtctgggtttcaccagaaagctctccagagggcccagaaagcagagaatatgttggagactcttcagggtcagctgacacacctggaggcagagctggtttctggaggtgttttgcgagacaacttgaattttgagaaacaaaaatatcttaaatttctggatcagctttctcagaaaatgaagttggaccagatggctgccgaacttggctttgacatgcggctggacgtggttttagctcgaacagagcagctggttcgtcttgagagcaatgcagtcattgagaacaagaccattgcccacaatttgcagagaaagctaaagacacagaaagagagactggagagcaaagaattacacatgagcctcctccggcagaaaatagcccagctggaggaggagaagcaggcacgcacggccttggtggttgagagggacaacgcgcatcttaccatcaggaacttgcagaagaaggtggagaggctgcagaaagagctgaacacgtgtcgagacttgcacaccgagctcaaagccaaactggccgacaccaatgaactgaagattaaaactttggaacagactaaagccattgaagatctaaacaaatccagagaccaactggagaagatgaaggagaaagctgagaaaaagctcatgtctgtcaagtcagaactggataccacagaacatgaggctaaggagaataaagaaagggccagaaacatgatagaagtggtaaccagtgaaatgaagacactaaaaaaatctctggaagaagcagaaaagagagaaaagcagctggcagacttcagggaggtggtgtcgcagatgctaggcttgaacgtgaccagccttgctcttcctgattatgaaatcatcaagtgtcttgaaagattggtccattcacatcagcatcactttgttacctgtgcctgcctcaaagatgtgactactgggcaagagaggcacccacaaggccatttacagcttcttcattgaacactgtatctcttgagagaggtggccataagacatggcacacaattcccaatttcacaaattcctcatgtctttgagatttgatcagtttgtgaatattttatgctttgatgatatagtgagaatgcatcacttgcaaaaacgatctcaaaagtgtcagccttagataaacgttcagcattaaaaacgcctattatttcatttactagcattttaggatccagaagaattccaccagattgcatgagttagattgggaaatgggagtgggagataatattgggaggtatctattttaagtcaggggctttactagccgatttagttctcacaataaccatgtggagaagctgtgacatttttaatttacaacctttctggggctcagacataaagttacctatccaaggttgcagttgggtagtggtgggaccaggatggacaactcattggccctgcctcaaaagccatacctcttctcctgctatgcagaatctgtttctcctgaatctctgtgatgctggtgggaattgtttgcatagaggaaggacaataaccctgccatcgtgagttaatgtccgggctggtcacagtggttcatgcctgtaatcccagcactttgggagtccaaggcaggcatatcatttgaggtcaggagtttaagaccagcctggctaacatagtgagaccctgtttctactaaaaatacaaaaataagccaggtgtggtggtgcatgactgtaatcccaactactcagcaggagaagcacttgaacccaggagacggaggcggcagtgagccaagattgtgccactgcactccagcctgggcgacagagtgaaactacatctcagaaaaaaaaaaaaaaaaaaaaaaaaaaagttaatgtccaaaaatgacagatttacaagtgtaagctatatgatttcttcaaaaagcaaaagcaatatacctaattcatttggatcaaacttacataggtctcaggtcctgtaagaaacttgcctgttctaactgttgctaccagattatatctggtggtaattgttaatgtttcagcagggctggtctcagtcctttaaaatggaaagctttatttggagcccaaccctataagatgaagaaatcctatatagtcttattcaccaatatatccaatacacccacagcaatggtacctttttaagatcaggattttattatgaattcctgtcactttctgttttccatttaaatttctattttacaaatttttcagggaatcatattcttaacttcactgagaaaaatgtgttactcttttggacaatttatcttatttctatcatataagatgtatttttttattgtccttaaaagaagctctagcatgaaattaaaggaaagggaaagaaatagatctggtgcacccgaacattaggagaaaatgaaaaatatacaaccaaccgttcgtgagtcatcaaaaagtcaaagtcagcctggccaacatggcaaaactccgtctctgcaaaaaatacaaaaatgagcccggtatgttggcatatgcctgtaatcccagctactcgagaggctgagacacgaaaattgcttgaacctgggaggcggaggttgcaatgagccgagatcgcgctactgcactccagcctgggcaacagagagagaccttgtctcaaaaaacaacaacaacaaaaagtcaaagtcataataagcaaattattggcttctttcttctagactaaaagaaattaaagagatgaaacaatcaattgcaagggtcaaaactagattggatcttggtttgaatgaaaaaaagcataaaatattcttgcaataattgtaaaaatttgaatgtggactaagtcctagattatattaaaatatttttaattttttaagcttgacaaatgcactgattgttatactttaaataactaaaaatctgagaatccacagtgctacagacaataagtgataaaatgggaaaaaatagtgaagatcttcttttaggaaaactaccaataaatgtagaatggatgattccaaaaaaaaaaaaaaaaagaagaaagagagaaagaaagaaacagaaaaacctaaaagaaagcgaatgaatttgacacctgttggttggatgacagaccacacagagggagagtgaccaggctttgcttgtggagcgggcatccctgcctagctatctccttcatgtgcaaggacagtataagtagtattttgcatgtgagtaaacacacctgcgcagccctggctgaggtcactaggctggccagaggcaggacaggcagtcttgaatttcctctaggggaggcctgcagttttccccaccaaccccttttcacatgctccaaagatgtagtagtgtctgctgttttggatcgaaaatcaccttgagtggaggaagtgacttcactgggtctctggaggctctcggagcttgagtggctctgccccaccctgaatcatgcacccataaatgcagttatgggtggatgtgggtgtatgagagtggagattgtttttgtacattctctttgttttcattttccaaatataatctttaataaacttgatttagtttcttgggaaatttattttctacgctattcaaagatattttaaataatattttaaaaacatgaaatttttttttcatttttgatttgatatcattaattttatatcaggattcatgtaagagactaaaaatgatccatagtggttagtactgtaatttctgtttattacatgaaattacattccatttatccaaatatatgtttatataatatttccactgtgtgactctagtgatctttaacatacacagaatgatctacagtgatctttaacatactcagaaatatgaaaaatgtttgaatatgatctttagggacttctaatgaaaagggtatatgaaatgggaacaataaattctgtacatgtatacagtccatatacacattaagtgtttgtcatttggacaaattgaaaacttttaataaagccattttcttctttgttaaaatgcca

SEQ ID NO: 37 - DNA, 4906 bp, Homo sapiens
atggtcataacagcctcctgtctaccgactcagaacggattttaccaaaactgaaaatgcaggctccatgctcagaagctctttaacaggctcgaaaggtccatgctcctttctcctgcccattctatagcataagaagacagtctctgagtgataatcttctcttcaagaagaagaaaactaggaaggagtaagcacaaagatctcttcacattctccgggactgcggtaccaaatatcagcacagcacttcttgaaaaaggatgtagattttaatctgaactttgaaccatcactgagcttagagacctgcgcaaagaaaatgaattcgtgaaaggacaaattgttattcttgaagagactataaatgtccatgagatggaagcaaaagctagcagagaaacgatcatgaggctggcttcagaagtcaacagagagcagaaaaaagctgcctcctgtactgaagagaaagagaagctgaaccaggacctgctcagtgctgtagaagcaaaagaagctcttgaaagggaagttaagatcttccaagaaaggctgcttgctggccagcaggtctgggatgcctcaaagcaggaagtgagcctcctgaagaaaagctcttctgagttggagaagagtttgaaggccagtcaggatgcagtcacaacctcacaaagccagtacttctcatttagggagaaaatcgcagccctccttaggggcagattgagcatgactgggtccactgaggacaccattttggagaagattcgagaaatggacagccgggaagaaagcagggaccggatggtctcccagcttgaagcccaaatatctgagcttgttgaacagttgggaaaggagtctgggtttcaccagaaagctctccagagggcccagaaagcagagaatatgttggagactcttcagggtcagctgacacacctggaggcagagctggtttctggaggtgttttgcgagacaacttgaattttgagaaacaaaaatatcttaaatttctggatcagctttctcagaaaatgaagttggaccagatggctgccgaacttggctttgacatgcggctggacgtggttttagctcgaacagagcagctggttcgtcttgagagcaatgcagtcattgagaacaagaccattgcccacaatttgcagagaaagctaaagacacagaaagagagactggagagcaaagaattacacatgagcctcctccggcagaaaatagcccagctggaggaggagaagcaggcacgcacggccttggtggttgagagggacaacgcgcatcttaccatcaggaacttgcagaagaaggtggagaggctgcagaaagagctgaacacgtgtcgagacttgcacaccgagctcaaagccaaactggccgacaccaatgaactgaagattaaaactttggaacagactaaagccattgaagatctaaacaaatccagagaccaactggagaagatgaaggagaaagctgagaaaaagctcatgtctgtcaagtcagaactggataccacagaacatgaggctaaggagaataaagaaagggccagaaacatgatagaagtggtaaccagtgaaatgaagacactaaaaaaatctctggaagaagcagaaaagagagaaaagcagctggcagacttcagggaggtggtgtcgcagatgctaggcttgaacgtgaccagccttgctcttcctgattatgaaatcatcaagtgtcttgaaagattggtccattcacatcagcatcactttgttacctgtgcctgcctcaaagatgtgactactgggcaagagaggcacccacaaggccatttacagcttcttcattgaacactgtatctcttgagagaggtggccataagacatggcacacaattcccaatttcacaaattcctcatgtctttgagatttgatcagtttgtgaatattttatgctttgatgatatagtgagaatgcatcacttgcaaaaacgatctcaaaagtgtcagccttagataaacgttcagcattaaaaacgcctattatttcatttactagcattttaggatccagaagaattccaccagattgcatgagttagattgggaaatgggagtgggagataatattgggaggtatctattttaagtcaggggctttactagccgatttagttctcacaataaccatgtggagaagctgtgacatttttaatttacaacctttctggggctcagacataaagttacctatccaaggttgcagttgggtagtggtgggaccaggatggacaactcattggccctgcctcaaaagccatacctcttctcctgctatgcagaatctgtttctcctgaatctctgtgatgctggtgggaattgtttgcatagaggaaggacaataaccctgccatcgtgagttaatgtccgggctggtcacagtggttcatgcctgtaatcccagcactttgggagtccaaggcaggcatatcatttgaggtcaggagtttaagaccagcctggctaacatagtgagaccctgtttctactaaaaatacaaaaataagccaggtgtggtggtgcatgactgtaatcccaactactcagcaggagaagcacttgaacccaggagacggaggcggcagtgagccaagattgtgccactgcactccagcctgggcgacagagtgaaactacatctcagaaaaaaaaaaaaaaaaaaaaaaaaaaagttaatgtccaaaaatgacagatttacaagtgtaagctatatgatttcttcaaaaagcaaaagcaatatacctaattcatttggatcaaacttacataggtctcaggtcctgtaagaaacttgcctgttctaactgttgctaccagattatatctggtggtaattgttaatgtttcagcagggctggtctcagtcctttaaaatggaaagctttatttggagcccaaccctataagatgaagaaatcctatatagtcttattcaccaatatatccaatacacccacagcaatggtacctttttaagatcaggattttattatgaattcctgtcactttctgttttccatttaaatttctattttacaaatttttcagggaatcatattcttaacttcactgagaaaaatgtgttactcttttggacaatttatcttatttctatcatataagatgtatttttttattgtccttaaaagaagctctagcatgaaattaaaggaaagggaaagaaatagatctggtgcacccgaacattaggagaaaatgaaaaatatacaaccaaccgttcgtgagtcatcaaaaagtcaaagtcagcctggccaacatggcaaaactccgtctctgcaaaaaatacaaaaatgagcccggtatgttggcatatgcctgtaatcccagctactcgagaggctgagacacgaaaattgcttgaacctgggaggcggaggttgcaatgagccgagatcgcgctactgcactccagcctgggcaacagagagagaccttgtctcaaaaaacaacaacaacaaaaagtcaaagtcataataagcaaattattggcttctttcttctagactaaaagaaattaaagagatgaaacaatcaattgcaagggtcaaaactagattggatcttggtttgaatgaaaaaaagcataaaatattcttgcaataattgtaaaaatttgaatgtggactaagtcctagattatattaaaatatttttaattttttaagcttgacaaatgcactgattgttatactttaaataactaaaaatctgagaatccacagtgctacagacaataagtgataaaatgggaaaaaatagtgaagatcttcttttaggaaaactaccaataaatgtagaatggatgattccaaaaaaaaaaaaaaaaagaagaaagagagaaagaaagaaacagaaaaacctaaaagaaagcgaatgaatttgacacctgttggttggatgacagaccacacagagggagagtgaccaggctttgcttgtggagcgggcatccctgcctagctatctccttcatgtgcaaggacagtataagtagtattttgcatgtgagtaaacacacctgcgcagccctggctgaggtcactaggctggccagaggcaggacaggcagtcttgaatttcctctaggggaggcctgcagttttccccaccaaccccttttcacatgctccaaagatgtagtagtgtctgctgttttggatcgaaaatcaccttgagtggaggaagtgacttcactgggtctctggaggctctcggagcttgagtggctctgccccaccctgaatcatgcacccataaatgcagttatgggtggatgtgggtgtatgagagtggagattgtttttgtacattctctttgttttcattttccaaatataatctttaataaacttgatttagtttcttgggaaatttattttctacgctattcaaagatattttaaataatattttaaaaacatgaaatttttttttcatttttgatttgatatcattaattttatatcaggattcatgtaagagactaaaaatgatccatagtggttagtactgtaatttctgtttattacatgaaattacattccatttatccaaatatatgtttatataatatttccactgtgtgactctagtgatctttaacatacacagaatgatctacagtgatctttaacatactcagaaatatgaaaaatgtttgaatatgatctttagggacttctaatgaaaagggtatatgaaatgggaacaataaattctgtacatgtatacagtccatatacacattaagtgtttgtcatttggacaaattgaaaacttttaataaagccattttcttctttgttaaaatgcca

SEQ ID NO: 38 - DNA, 4720 bp, Homo sapiens
atggtcataacagcctcctgtctaccgactcagaacggattttaccaaaactgaaaatgcaggctccatgctcagaagctctttaacaggctcgaaaggtccatgctcctttctcctgcccattctatagcataagaagacagtctctgagtgataatcttctcttcaagaagaagaaaactaggaaggagtaagcacaaagatctcttcacattctccgggactgcggtaccaaatatcagcacagcacttcttgaaaaaggatgtagattttaatctgaactttgaaccatcactgaggacctgctcagtgctgtagaagcaaaagaagctcttgaaagggaagttaagatcttccaagaaaggctgcttgctggccagcaggtctgggatgcctcaaagcaggaagtgagcctcctgaagaaaagctcttctgagttggagaagagtttgaaggccagtcaggatgcagtcacaacctcacaaagccagtacttctcatttagggagaaaatcgcagccctccttaggggcagattgagcatgactgggtccactgaggacaccattttggagaagattcgagaaatggacagccgggaagaaagcagggaccggatggtctcccagcttgaagcccaaatatctgagcttgttgaacagttgggaaaggagtctgggtttcaccagaaagctctccagagggcccagaaagcagagaatatgttggagactcttcagggtcagctgacacacctggaggcagagctggtttctggaggtgttttgcgagacaacttgaattttgagaaacaaaaatatcttaaatttctggatcagctttctcagaaaatgaagttggaccagatggctgccgaacttggctttgacatgcggctggacgtggttttagctcgaacagagcagctggttcgtcttgagagcaatgcagtcattgagaacaagaccattgcccacaatttgcagagaaagctaaagacacagaaagagagactggagagcaaagaattacacatgagcctcctccggcagaaaatagcccagctggaggaggagaagcaggcacgcacggccttggtggttgagagggacaacgcgcatcttaccatcaggaacttgcagaagaaggtggagaggctgcagaaagagctgaacacgtgtcgagacttgcacaccgagctcaaagccaaactggccgacaccaatgaactgaagattaaaactttggaacagactaaagccattgaagatctaaacaaatccagagaccaactggagaagatgaaggagaaagctgagaaaaagctcatgtctgtcaagtcagaactggataccacagaacatgaggctaaggagaataaagaaagggccagaaacatgatagaagtggtaaccagtgaaatgaagacactaaaaaaatctctggaagaagcagaaaagagagaaaagcagctggcagacttcagggaggtggtgtcgcagatgctaggcttgaacgtgaccagccttgctcttcctgattatgaaatcatcaagtgtcttgaaagattggtccattcacatcagcatcactttgttacctgtgcctgcctcaaagatgtgactactgggcaagagaggcacccacaaggccatttacagcttcttcattgaacactgtatctcttgagagaggtggccataagacatggcacacaattcccaatttcacaaattcctcatgtctttgagatttgatcagtttgtgaatattttatgctttgatgatatagtgagaatgcatcacttgcaaaaacgatctcaaaagtgtcagccttagataaacgttcagcattaaaaacgcctattatttcatttactagcattttaggatccagaagaattccaccagattgcatgagttagattgggaaatgggagtgggagataatattgggaggtatctattttaagtcaggggctttactagccgatttagttctcacaataaccatgtggagaagctgtgacatttttaatttacaacctttctggggctcagacataaagttacctatccaaggttgcagttgggtagtggtgggaccaggatggacaactcattggccctgcctcaaaagccatacctcttctcctgctatgcagaatctgtttctcctgaatctctgtgatgctggtgggaattgtttgcatagaggaaggacaataaccctgccatcgtgagttaatgtccgggctggtcacagtggttcatgcctgtaatcccagcactttgggagtccaaggcaggcatatcatttgaggtcaggagtttaagaccagcctggctaacatagtgagaccctgtttctactaaaaatacaaaaataagccaggtgtggtggtgcatgactgtaatcccaactactcagcaggagaagcacttgaacccaggagacggaggcggcagtgagccaagattgtgccactgcactccagcctgggcgacagagtgaaactacatctcagaaaaaaaaaaaaaaaaaaaaaaaaaaagttaatgtccaaaaatgacagatttacaagtgtaagctatatgatttcttcaaaaagcaaaagcaatatacctaattcatttggatcaaacttacataggtctcaggtcctgtaagaaacttgcctgttctaactgttgctaccagattatatctggtggtaattgttaatgtttcagcagggctggtctcagtcctttaaaatggaaagctttatttggagcccaaccctataagatgaagaaatcctatatagtcttattcaccaatatatccaatacacccacagcaatggtacctttttaagatcaggattttattatgaattcctgtcactttctgttttccatttaaatttctattttacaaatttttcagggaatcatattcttaacttcactgagaaaaatgtgttactcttttggacaatttatcttatttctatcatataagatgtatttttttattgtccttaaaagaagctctagcatgaaattaaaggaaagggaaagaaatagatctggtgcacccgaacattaggagaaaatgaaaaatatacaaccaaccgttcgtgagtcatcaaaaagtcaaagtcagcctggccaacatggcaaaactccgtctctgcaaaaaatacaaaaatgagcccggtatgttggcatatgcctgtaatcccagctactcgagaggctgagacacgaaaattgcttgaacctgggaggcggaggttgcaatgagccgagatcgcgctactgcactccagcctgggcaacagagagagaccttgtctcaaaaaacaacaacaacaaaaagtcaaagtcataataagcaaattattggcttctttcttctagactaaaagaaattaaagagatgaaacaatcaattgcaagggtcaaaactagattggatcttggtttgaatgaaaaaaagcataaaatattcttgcaataattgtaaaaatttgaatgtggactaagtcctagattatattaaaatatttttaattttttaagcttgacaaatgcactgattgttatactttaaataactaaaaatctgagaatccacagtgctacagacaataagtgataaaatgggaaaaaatagtgaagatcttcttttaggaaaactaccaataaatgtagaatggatgattccaaaaaaaaaaaaaaaaagaagaaagagagaaagaaagaaacagaaaaacctaaaagaaagcgaatgaatttgacacctgttggttggatgacagaccacacagagggagagtgaccaggctttgcttgtggagcgggcatccctgcctagctatctccttcatgtgcaaggacagtataagtagtattttgcatgtgagtaaacacacctgcgcagccctggctgaggtcactaggctggccagaggcaggacaggcagtcttgaatttcctctaggggaggcctgcagttttccccaccaaccccttttcacatgctccaaagatgtagtagtgtctgctgttttggatcgaaaatcaccttgagtggaggaagtgacttcactgggtctctggaggctctcggagcttgagtggctctgccccaccctgaatcatgcacccataaatgcagttatgggtggatgtgggtgtatgagagtggagattgtttttgtacattctctttgttttcattttccaaatataatctttaataaacttgatttagtttcttgggaaatttattttctacgctattcaaagatattttaaataatattttaaaaacatgaaatttttttttcatttttgatttgatatcattaattttatatcaggattcatgtaagagactaaaaatgatccatagtggttagtactgtaatttctgtttattacatgaaattacattccatttatccaaatatatgtttatataatatttccactgtgtgactctagtgatctttaacatacacagaatgatctacagtgatctttaacatactcagaaatatgaaaaatgtttgaatatgatctttagggacttctaatgaaaagggtatatgaaatgggaacaataaattctgtacatgtatacagtccatatacacattaagtgtttgtcatttggacaaattgaaaacttttaataaagccattttcttctttgttaaaatgcca

SEQ ID NO: 39 - DNA, 4402 bp, Homo sapiens
atggtcataacagcctcctgtctaccgactcagaacggattttaccaaaactgaaaatgcaggctccatgctcagaagctctttaacaggctcgaaaggtccatgctcctttctcctgcccattctatagcataagaagacagtctctgagtgataatcttctcttcaagaagaagaaaactaggaaggagtaagcacaaagatctcttcacattctccgggactgcggtaccaaatatcagcacagcacttcttgaaaaaggatgtagattttaatctgaactttgaaccatcactgagatggtctcccagcttgaagcccaaatatctgagcttgttgaacagttgggaaaggagtctgggtttcaccagaaagctctccagagggcccagaaagcagagaatatgttggagactcttcagggtcagctgacacacctggaggcagagctggtttctggaggtgttttgcgagacaacttgaattttgagaaacaaaaatatcttaaatttctggatcagctttctcagaaaatgaagttggaccagatggctgccgaacttggctttgacatgcggctggacgtggttttagctcgaacagagcagctggttcgtcttgagagcaatgcagtcattgagaacaagaccattgcccacaatttgcagagaaagctaaagacacagaaagagagactggagagcaaagaattacacatgagcctcctccggcagaaaatagcccagctggaggaggagaagcaggcacgcacggccttggtggttgagagggacaacgcgcatcttaccatcaggaacttgcagaagaaggtggagaggctgcagaaagagctgaacacgtgtcgagacttgcacaccgagctcaaagccaaactggccgacaccaatgaactgaagattaaaactttggaacagactaaagccattgaagatctaaacaaatccagagaccaactggagaagatgaaggagaaagctgagaaaaagctcatgtctgtcaagtcagaactggataccacagaacatgaggctaaggagaataaagaaagggccagaaacatgatagaagtggtaaccagtgaaatgaagacactaaaaaaatctctggaagaagcagaaaagagagaaaagcagctggcagacttcagggaggtggtgtcgcagatgctaggcttgaacgtgaccagccttgctcttcctgattatgaaatcatcaagtgtcttgaaagattggtccattcacatcagcatcactttgttacctgtgcctgcctcaaagatgtgactactgggcaagagaggcacccacaaggccatttacagcttcttcattgaacactgtatctcttgagagaggtggccataagacatggcacacaattcccaatttcacaaattcctcatgtctttgagatttgatcagtttgtgaatattttatgctttgatgatatagtgagaatgcatcacttgcaaaaacgatctcaaaagtgtcagccttagataaacgttcagcattaaaaacgcctattatttcatttactagcattttaggatccagaagaattccaccagattgcatgagttagattgggaaatgggagtgggagataatattgggaggtatctattttaagtcaggggctttactagccgatttagttctcacaataaccatgtggagaagctgtgacatttttaatttacaacctttctggggctcagacataaagttacctatccaaggttgcagttgggtagtggtgggaccaggatggacaactcattggccctgcctcaaaagccatacctcttctcctgctatgcagaatctgtttctcctgaatctctgtgatgctggtgggaattgtttgcatagaggaaggacaataaccctgccatcgtgagttaatgtccgggctggtcacagtggttcatgcctgtaatcccagcactttgggagtccaaggcaggcatatcatttgaggtcaggagtttaagaccagcctggctaacatagtgagaccctgtttctactaaaaatacaaaaataagccaggtgtggtggtgcatgactgtaatcccaactactcagcaggagaagcacttgaacccaggagacggaggcggcagtgagccaagattgtgccactgcactccagcctgggcgacagagtgaaactacatctcagaaaaaaaaaaaaaaaaaaaaaaaaaaagttaatgtccaaaaatgacagatttacaagtgtaagctatatgatttcttcaaaaagcaaaagcaatatacctaattcatttggatcaaacttacataggtctcaggtcctgtaagaaacttgcctgttctaactgttgctaccagattatatctggtggtaattgttaatgtttcagcagggctggtctcagtcctttaaaatggaaagctttatttggagcccaaccctataagatgaagaaatcctatatagtcttattcaccaatatatccaatacacccacagcaatggtacctttttaagatcaggattttattatgaattcctgtcactttctgttttccatttaaatttctattttacaaatttttcagggaatcatattcttaacttcactgagaaaaatgtgttactcttttggacaatttatcttatttctatcatataagatgtatttttttattgtccttaaaagaagctctagcatgaaattaaaggaaagggaaagaaatagatctggtgcacccgaacattaggagaaaatgaaaaatatacaaccaaccgttcgtgagtcatcaaaaagtcaaagtcagcctggccaacatggcaaaactccgtctctgcaaaaaatacaaaaatgagcccggtatgttggcatatgcctgtaatcccagctactcgagaggctgagacacgaaaattgcttgaacctgggaggcggaggttgcaatgagccgagatcgcgctactgcactccagcctgggcaacagagagagaccttgtctcaaaaaacaacaacaacaaaaagtcaaagtcataataagcaaattattggcttctttcttctagactaaaagaaattaaagagatgaaacaatcaattgcaagggtcaaaactagattggatcttggtttgaatgaaaaaaagcataaaatattcttgcaataattgtaaaaatttgaatgtggactaagtcctagattatattaaaatatttttaattttttaagcttgacaaatgcactgattgttatactttaaataactaaaaatctgagaatccacagtgctacagacaataagtgataaaatgggaaaaaatagtgaagatcttcttttaggaaaactaccaataaatgtagaatggatgattccaaaaaaaaaaaaaaaaagaagaaagagagaaagaaagaaacagaaaaacctaaaagaaagcgaatgaatttgacacctgttggttggatgacagaccacacagagggagagtgaccaggctttgcttgtggagcgggcatccctgcctagctatctccttcatgtgcaaggacagtataagtagtattttgcatgtgagtaaacacacctgcgcagccctggctgaggtcactaggctggccagaggcaggacaggcagtcttgaatttcctctaggggaggcctgcagttttccccaccaaccccttttcacatgctccaaagatgtagtagtgtctgctgttttggatcgaaaatcaccttgagtggaggaagtgacttcactgggtctctggaggctctcggagcttgagtggctctgccccaccctgaatcatgcacccataaatgcagttatgggtggatgtgggtgtatgagagtggagattgtttttgtacattctctttgttttcattttccaaatataatctttaataaacttgatttagtttcttgggaaatttattttctacgctattcaaagatattttaaataatattttaaaaacatgaaatttttttttcatttttgatttgatatcattaattttatatcaggattcatgtaagagactaaaaatgatccatagtggttagtactgtaatttctgtttattacatgaaattacattccatttatccaaatatatgtttatataatatttccactgtgtgactctagtgatctttaacatacacagaatgatctacagtgatctttaacatactcagaaatatgaaaaatgtttgaatatgatctttagggacttctaatgaaaagggtatatgaaatgggaacaataaattctgtacatgtatacagtccatatacacattaagtgtttgtcatttggacaaattgaaaacttttaataaagccattttcttctttgttaaaatgcca

SEQ ID NO: 40 - DNA, 4201 bp, Homo sapiens
atggtcataacagcctcctgtctaccgactcagaacggattttaccaaaactgaaaatgcaggctccatgctcagaagctctttaacaggctcgaaaggtccatgctcctttctcctgcccattctatagcataagaagacagtctctgagtgataatcttctcttcaagaagaagaaaactaggaaggagtaagcacaaagatctcttcacattctccgggactgcggtaccaaatatcagcacagcacttcttgaaaaaggatgtagattttaatctgaactttgaaccatcactgagtatcttaaatttctggatcagctttctcagaaaatgaagttggaccagatggctgccgaacttggctttgacatgcggctggacgtggttttagctcgaacagagcagctggttcgtcttgagagcaatgcagtcattgagaacaagaccattgcccacaatttgcagagaaagctaaagacacagaaagagagactggagagcaaagaattacacatgagcctcctccggcagaaaatagcccagctggaggaggagaagcaggcacgcacggccttggtggttgagagggacaacgcgcatcttaccatcaggaacttgcagaagaaggtggagaggctgcagaaagagctgaacacgtgtcgagacttgcacaccgagctcaaagccaaactggccgacaccaatgaactgaagattaaaactttggaacagactaaagccattgaagatctaaacaaatccagagaccaactggagaagatgaaggagaaagctgagaaaaagctcatgtctgtcaagtcagaactggataccacagaacatgaggctaaggagaataaagaaagggccagaaacatgatagaagtggtaaccagtgaaatgaagacactaaaaaaatctctggaagaagcagaaaagagagaaaagcagctggcagacttcagggaggtggtgtcgcagatgctaggcttgaacgtgaccagccttgctcttcctgattatgaaatcatcaagtgtcttgaaagattggtccattcacatcagcatcactttgttacctgtgcctgcctcaaagatgtgactactgggcaagagaggcacccacaaggccatttacagcttcttcattgaacactgtatctcttgagagaggtggccataagacatggcacacaattcccaatttcacaaattcctcatgtctttgagatttgatcagtttgtgaatattttatgctttgatgatatagtgagaatgcatcacttgcaaaaacgatctcaaaagtgtcagccttagataaacgttcagcattaaaaacgcctattatttcatttactagcattttaggatccagaagaattccaccagattgcatgagttagattgggaaatgggagtgggagataatattgggaggtatctattttaagtcaggggctttactagccgatttagttctcacaataaccatgtggagaagctgtgacatttttaatttacaacctttctggggctcagacataaagttacctatccaaggttgcagttgggtagtggtgggaccaggatggacaactcattggccctgcctcaaaagccatacctcttctcctgctatgcagaatctgtttctcctgaatctctgtgatgctggtgggaattgtttgcatagaggaaggacaataaccctgccatcgtgagttaatgtccgggctggtcacagtggttcatgcctgtaatcccagcactttgggagtccaaggcaggcatatcatttgaggtcaggagtttaagaccagcctggctaacatagtgagaccctgtttctactaaaaatacaaaaataagccaggtgtggtggtgcatgactgtaatcccaactactcagcaggagaagcacttgaacccaggagacggaggcggcagtgagccaagattgtgccactgcactccagcctgggcgacagagtgaaactacatctcagaaaaaaaaaaaaaaaaaaaaaaaaaaagttaatgtccaaaaatgacagatttacaagtgtaagctatatgatttcttcaaaaagcaaaagcaatatacctaattcatttggatcaaacttacataggtctcaggtcctgtaagaaacttgcctgttctaactgttgctaccagattatatctggtggtaattgttaatgtttcagcagggctggtctcagtcctttaaaatggaaagctttatttggagcccaaccctataagatgaagaaatcctatatagtcttattcaccaatatatccaatacacccacagcaatggtacctttttaagatcaggattttattatgaattcctgtcactttctgttttccatttaaatttctattttacaaatttttcagggaatcatattcttaacttcactgagaaaaatgtgttactcttttggacaatttatcttatttctatcatataagatgtatttttttattgtccttaaaagaagctctagcatgaaattaaaggaaagggaaagaaatagatctggtgcacccgaacattaggagaaaatgaaaaatatacaaccaaccgttcgtgagtcatcaaaaagtcaaagtcagcctggccaacatggcaaaactccgtctctgcaaaaaatacaaaaatgagcccggtatgttggcatatgcctgtaatcccagctactcgagaggctgagacacgaaaattgcttgaacctgggaggcggaggttgcaatgagccgagatcgcgctactgcactccagcctgggcaacagagagagaccttgtctcaaaaaacaacaacaacaaaaagtcaaagtcataataagcaaattattggcttctttcttctagactaaaagaaattaaagagatgaaacaatcaattgcaagggtcaaaactagattggatcttggtttgaatgaaaaaaagcataaaatattcttgcaataattgtaaaaatttgaatgtggactaagtcctagattatattaaaatatttttaattttttaagcttgacaaatgcactgattgttatactttaaataactaaaaatctgagaatccacagtgctacagacaataagtgataaaatgggaaaaaatagtgaagatcttcttttaggaaaactaccaataaatgtagaatggatgattccaaaaaaaaaaaaaaaaagaagaaagagagaaagaaagaaacagaaaaacctaaaagaaagcgaatgaatttgacacctgttggttggatgacagaccacacagagggagagtgaccaggctttgcttgtggagcgggcatccctgcctagctatctccttcatgtgcaaggacagtataagtagtattttgcatgtgagtaaacacacctgcgcagccctggctgaggtcactaggctggccagaggcaggacaggcagtcttgaatttcctctaggggaggcctgcagttttccccaccaaccccttttcacatgctccaaagatgtagtagtgtctgctgttttggatcgaaaatcaccttgagtggaggaagtgacttcactgggtctctggaggctctcggagcttgagtggctctgccccaccctgaatcatgcacccataaatgcagttatgggtggatgtgggtgtatgagagtggagattgtttttgtacattctctttgttttcattttccaaatataatctttaataaacttgatttagtttcttgggaaatttattttctacgctattcaaagatattttaaataatattttaaaaacatgaaatttttttttcatttttgatttgatatcattaattttatatcaggattcatgtaagagactaaaaatgatccatagtggttagtactgtaatttctgtttattacatgaaattacattccatttatccaaatatatgtttatataatatttccactgtgtgactctagtgatctttaacatacacagaatgatctacagtgatctttaacatactcagaaatatgaaaaatgtttgaatatgatctttagggacttctaatgaaaagggtatatgaaatgggaacaataaattctgtacatgtatacagtccatatacacattaagtgtttgtcatttggacaaattgaaaacttttaataaagccattttcttctttgttaaaatgcca

SEQ ID NO: 41 - DNA, 3784 bp, Homo sapiens
atggtcataacagcctcctgtctaccgactcagaacggattttaccaaaactgaaaatgcaggctccatgctcagaagctctttaacaggctcgaaaggtccatgctcctttctcctgcccattctatagcataagaagacagtctctgagtgataatcttctcttcaagaagaagaaaactaggaaggagtaagcacaaagatctcttcacattctccgggactgcggtaccaaatatcagcacagcacttcttgaaaaaggatgtagattttaatctgaactttgaaccatcactgagattaaaactttggaacagactaaagccattgaagatctaaacaaatccagagaccaactggagaagatgaaggagaaagctgagaaaaagctcatgtctgtcaagtcagaactggataccacagaacatgaggctaaggagaataaagaaagggccagaaacatgatagaagtggtaaccagtgaaatgaagacactaaaaaaatctctggaagaagcagaaaagagagaaaagcagctggcagacttcagggaggtggtgtcgcagatgctaggcttgaacgtgaccagccttgctcttcctgattatgaaatcatcaagtgtcttgaaagattggtccattcacatcagcatcactttgttacctgtgcctgcctcaaagatgtgactactgggcaagagaggcacccacaaggccatttacagcttcttcattgaacactgtatctcttgagagaggtggccataagacatggcacacaattcccaatttcacaaattcctcatgtctttgagatttgatcagtttgtgaatattttatgctttgatgatatagtgagaatgcatcacttgcaaaaacgatctcaaaagtgtcagccttagataaacgttcagcattaaaaacgcctattatttcatttactagcattttaggatccagaagaattccaccagattgcatgagttagattgggaaatgggagtgggagataatattgggaggtatctattttaagtcaggggctttactagccgatttagttctcacaataaccatgtggagaagctgtgacatttttaatttacaacctttctggggctcagacataaagttacctatccaaggttgcagttgggtagtggtgggaccaggatggacaactcattggccctgcctcaaaagccatacctcttctcctgctatgcagaatctgtttctcctgaatctctgtgatgctggtgggaattgtttgcatagaggaaggacaataaccctgccatcgtgagttaatgtccgggctggtcacagtggttcatgcctgtaatcccagcactttgggagtccaaggcaggcatatcatttgaggtcaggagtttaagaccagcctggctaacatagtgagaccctgtttctactaaaaatacaaaaataagccaggtgtggtggtgcatgactgtaatcccaactactcagcaggagaagcacttgaacccaggagacggaggcggcagtgagccaagattgtgccactgcactccagcctgggcgacagagtgaaactacatctcagaaaaaaaaaaaaaaaaaaaaaaaaaaagttaatgtccaaaaatgacagatttacaagtgtaagctatatgatttcttcaaaaagcaaaagcaatatacctaattcatttggatcaaacttacataggtctcaggtcctgtaagaaacttgcctgttctaactgttgctaccagattatatctggtggtaattgttaatgtttcagcagggctggtctcagtcctttaaaatggaaagctttatttggagcccaaccctataagatgaagaaatcctatatagtcttattcaccaatatatccaatacacccacagcaatggtacctttttaagatcaggattttattatgaattcctgtcactttctgttttccatttaaatttctattttacaaatttttcagggaatcatattcttaacttcactgagaaaaatgtgttactcttttggacaatttatcttatttctatcatataagatgtatttttttattgtccttaaaagaagctctagcatgaaattaaaggaaagggaaagaaatagatctggtgcacccgaacattaggagaaaatgaaaaatatacaaccaaccgttcgtgagtcatcaaaaagtcaaagtcagcctggccaacatggcaaaactccgtctctgcaaaaaatacaaaaatgagcccggtatgttggcatatgcctgtaatcccagctactcgagaggctgagacacgaaaattgcttgaacctgggaggcggaggttgcaatgagccgagatcgcgctactgcactccagcctgggcaacagagagagaccttgtctcaaaaaacaacaacaacaaaaagtcaaagtcataataagcaaattattggcttctttcttctagactaaaagaaattaaagagatgaaacaatcaattgcaagggtcaaaactagattggatcttggtttgaatgaaaaaaagcataaaatattcttgcaataattgtaaaaatttgaatgtggactaagtcctagattatattaaaatatttttaattttttaagcttgacaaatgcactgattgttatactttaaataactaaaaatctgagaatccacagtgctacagacaataagtgataaaatgggaaaaaatagtgaagatcttcttttaggaaaactaccaataaatgtagaatggatgattccaaaaaaaaaaaaaaaaagaagaaagagagaaagaaagaaacagaaaaacctaaaagaaagcgaatgaatttgacacctgttggttggatgacagaccacacagagggagagtgaccaggctttgcttgtggagcgggcatccctgcctagctatctccttcatgtgcaaggacagtataagtagtattttgcatgtgagtaaacacacctgcgcagccctggctgaggtcactaggctggccagaggcaggacaggcagtcttgaatttcctctaggggaggcctgcagttttccccaccaaccccttttcacatgctccaaagatgtagtagtgtctgctgttttggatcgaaaatcaccttgagtggaggaagtgacttcactgggtctctggaggctctcggagcttgagtggctctgccccaccctgaatcatgcacccataaatgcagttatgggtggatgtgggtgtatgagagtggagattgtttttgtacattctctttgttttcattttccaaatataatctttaataaacttgatttagtttcttgggaaatttattttctacgctattcaaagatattttaaataatattttaaaaacatgaaatttttttttcatttttgatttgatatcattaattttatatcaggattcatgtaagagactaaaaatgatccatagtggttagtactgtaatttctgtttattacatgaaattacattccatttatccaaatatatgtttatataatatttccactgtgtgactctagtgatctttaacatacacagaatgatctacagtgatctttaacatactcagaaatatgaaaaatgtttgaatatgatctttagggacttctaatgaaaagggtatatgaaatgggaacaataaattctgtacatgtatacagtccatatacacattaagtgtttgtcatttggacaaattgaaaacttttaataaagccattttcttctttgttaaaatgcca

SEQ ID NO: 42 - DNA, 22 bp, Artificial Sequence
  Synthetic construct
ctgcggtaccaaatatcagcac

SEQ ID NO: 43 - DNA, 22 bp, Artificial Sequence
  Synthetic construct
cttctccagttggtctctggat

SEQ ID NO: 44 - DNA, 22 bp, Artificial Sequence
  Synthetic construct
aacaggctcgaaaggtccatgc

SEQ ID NO: 45 - DNA, 22 bp, Artificial Sequence
  Synthetic construct
gcctagcatctgcgacaccacc